(19) United States
(12) Patent Application Publication
(10) Pub. No.: US 2026/0137414 A1
(43) Pub. Date: May 21, 2026

(54) DEVICES AND METHODS FOR CROSSING A VASCULAR OCCLUSION WITHOUT EXPANSION OF VESSEL WALL

(71) Applicant: AngioSafe, Inc., San Jose, CA (US)

(72) Inventors: Mallik THATIPELLI, Bakersfield, CA (US); Sarvajna Kumar DWIVEDI, Redwood City, CA (US)

(73) Assignee: AngioSafe, Inc., San Jose, CA (US)

(21) Appl. No.: 18/951,495

(22) Filed: Nov. 18, 2024

(51) Int. Cl. A61B 17/3207; A61B 17/22

(52) U.S. Cl. CPC A61B 17/3207; A61B 2017/22094

(57) ABSTRACT

Disclosed herein are devices and methods for tunneling through an occlusion. The devices can include a handle, a shaft, and a tip. The shaft can be surrounded by a centering cage. The tip can have one or more cutting loops. Tunneling can be done by drilling, cutting, penetrating, pulverizing, blunt dissection or any combination thereof. Tunneling can use a pushing force, rotation, or both. The vessel wall can avoid being stretched, extended, or expanded. The vessel wall can avoid being dissected, cut, perforated, or damaged.

### BACKGROUND

Chronic total occlusions (CTO) are vascular lesions that block most or all blood flow through a blood vessel. CTOs can occur in most blood vessels, including coronary arteries, carotid arteries, iliac arteries and veins, femoral arteries and veins, and popliteal arteries and veins. If not treated properly, CTOs can result in pain, loss of sensation, loss of use, amputation, heart attacks, or death.

### SUMMARY

Described herein are devices, systems, and methods for crossing chronic total occlusions.

Provided herein is a method for treating an occluded blood vessel. The method can comprise centering a working end of a treatment catheter in the occluded blood vessel with plaque and/or occlusion mass. The method can comprise disrupting the plaque and/or occlusion mass with the working end of the treatment catheter. In some cases, the method comprises radially displacing the disrupted plaque and/or occlusion mass and adjacent plaque and/or occlusion mass. Radially displacing the disrupted and adjacent plaque and/or occlusion mass may enlarge a diameter of a channel in the occluded blood vessel while simultaneously maintaining the centering of the working end of the treatment catheter. Radially displacing the disrupted and adjacent plaque and/or occlusion mass may enlarge the channel by compressing the disrupted and adjacent plaque and/or occlusion mass against a wall of the blood vessel while minimizing distension injury (barotrauma) to the blood vessel. The method may comprise withdrawing the treatment catheter from the blood vessel after the channel is enlarged. The channel can remain enlarged after the catheter is withdrawn.

Described herein is a treatment catheter device for treating an occluded blood vessel. The catheter can comprise a tubular catheter body having a distal end, a proximal end, and a central passage therethrough. In some cases, the catheter comprises a rotatable shaft extending through the central passage of the tubular catheter body and having a distal end, a proximal end, and a central lumen therethrough. The catheter can comprise a cutting tip mounted on the distal end of the rotatable shaft and configured to cut through occlusive material when rotated, pushed, or both. The cutting tip may have a passage contiguous with the central lumen of the rotatable shaft. The catheter may comprise at least one flat spring disposed circumferentially about a distal portion of the tubular catheter body and adapted to elastically self-expand from a radially constrained configuration to a radially expanded configuration. Each flat spring may have (1) a wide lateral surface configured to atraumatically engage against a wall region of the blood vessel to centrally align the tubular catheter body in a lumen of the blood vessel and (2) a narrow distal edge configured to penetrate the occlusion as the catheter is distally advanced. The catheter can comprise a handle at the proximal end of the tubular catheter body.

Provided herein is a treatment catheter for treating an occluded blood vessel. The catheter can comprise a tubular catheter body having a distal end, a proximal end, and a central passage therethrough. In some cases, the catheter comprises a rotatable shaft extending through the central passage of the tubular catheter body and having a distal end, a proximal end, and a central lumen therethrough. The catheter can comprise a cutting tip mounted on the distal end of the rotatable shaft and configured to cut through occlusive material when rotated, pushed, or both. The cutting tip may have a passage contiguous with the central lumen of the rotatable shaft. The catheter may comprise an expandable dilating element disposed circumferentially about a distal portion of the tubular catheter body distal to at least one centering and plaque and/or occlusion mass displacement element. The expandable dilating element may be configured to pre-dilate the occluded blood vessel before the at least one centering and plaque and/or occlusion mass displacement element engages the occluded blood vessel.

Described herein is a treatment catheter for treating an occluded blood vessel. In some cases, the catheter comprises a tubular catheter body having a distal end, a proximal end, and a central passage therethrough. The catheter can comprise a rotatable shaft extending through the central passage of the tubular catheter body and having a distal end, a proximal end, and a central lumen therethrough. The catheter may comprise an energy projecting element. The energy projecting element can be configured to disrupt occlusive material in the occluded blood vessel.

Provided herein is a treatment catheter for treating an occluded blood vessel. In some cases, the catheter comprises a tubular catheter body having a distal end, a proximal end, and a central passage therethrough. The catheter can comprise a cutting tip mounted on the distal end of the rotatable shaft and configured to cut through occlusive material when rotated, pushed, or both. The catheter can comprise at least one cutting loop mounted on a distal end of the cutting tip. In some cases, the cutting tip has a passage contiguous with the central lumen of the rotatable shaft. In some cases, the cutting tip and the at least one cutting loop are configured to centrally align the catheter in a lumen of the blood vessel. The catheter can comprise a handle at the proximal end of the rotatable shaft.

Described herein is a guiding catheter for accessing a target blood vessel in a patient's ascending aorta. The guiding catheter can comprise an elongated catheter body. The elongated body can comprise a proximal end and a distal end. The elongated catheter body can be configured to slidably receive an interventional catheter through the proximal end and guide the interventional catheter to the target blood vessel. In some cases, a distal tip of the elongate catheter body is configured to removably engage an ostium of the target blood vessel. In some cases, a distal region of the elongated catheter body is formed as a loop with a curve that bends and crosses over itself.

Provided herein is a method for accessing a target blood vessel in a patient's ascending aorta. The method can comprise advancing a distal region of an elongate catheter body into the patient's ascending aorta. In some cases, the distal region of the elongate catheter is constrained in a straightened configuration. The method can comprise releasing the distal region of the elongate catheter body from constraint. In some cases, the distal region forms a loop with a curve that bends and crosses over itself within the patient's ascending aorta. The method can comprise engaging a distal tip of the elongated catheter body against an ostium of the target blood vessel. The method may comprise advancing an interventional catheter through a lumen of the elongate catheter body to the ostium and into the target blood vessel.

Described herein is a catheter for treating an occluded blood vessel. The catheter can comprise a rotatable shaft having a distal end, a proximal end, and a central lumen therethrough. The catheter can comprise a cutting tip mounted on the distal end of the rotatable shaft. The cutting tip can be configured to cut through occlusive material when rotated, pushed, or both. In some cases, the cutting tip has a passage contiguous with the central lumen of the rotatable shaft. The catheter can comprise an expandable element disposed circumferentially about a distal portion of the rotatable shaft. In some cases, the expandable element is configured to be in a contracted state when the catheter is inserted into the blood vessel and in an expanded state when the catheter is cutting through the occlusive material. In some cases, the expandable element is configured to exert force to radially displace plaque and/or occlusion mass causing the occlusion substantially while minimizing distension of a wall of the blood vessel.

Provided herein is a catheter for treating an occluded blood vessel. The catheter can comprise a tubular catheter body having a distal end, a proximal end, and a central passage therethrough. The catheter can comprise a rotatable shaft extending through the central passage of the tubular catheter body and having a distal end, a proximal end, and a central lumen therethrough. The catheter can comprise a conical cutting tip mounted on the distal end of the rotatable shaft. The conical cutting tip can be configured to cut through occlusive material when rotated, pushed, or both. The cutting tip can comprise a helically cut surface. The cutting tip may have a passage contiguous with the central lumen of the rotatable shaft. The catheter can comprise a handle at the proximal end of the tubular catheter body.

Described herein is a system for treating an occluded blood vessel. The system can comprise a treatment catheter. The system can comprise a guidewire disposed inside the central lumen of the treatment catheter. In some cases, a distal end of the guidewire comprises a spiral cone filter configured to capture debris from crossing the occluded blood vessel. In some cases, a distal tip of the guidewire comprises a coil. The system can comprise an aspiration syringe. In some cases, the aspiration syringe is configured to collect the captured debris from the spiral cone filter.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIGS. 1A-1B show a cross-sectional view and a side view, respectively, captured with intravascular ultrasound (IVUS) of a channel in the vessel resulting from passage of a device as described herein as it crossed through using methods described herein.

FIG. 2A illustrates an embodiment of a device constructed in accordance with the embodiments described herein.

FIG. 2B shows an alternate handle of a device in accordance with embodiments described herein.

FIG. 2C shows an alternate handle of a device in accordance with embodiments described herein.

FIG. 3A depicts a perspective side view of the distal portion of the device of FIG. 1 shown with a centering cage in accordance with an embodiment described herein.

FIG. 3B illustrates an inner view of the device of FIG. 3A, thereby showing the inner view of the distal portion of FIG. 1 in accordance with embodiments described herein.

FIG. 3C illustrates a cross-sectional view of the device of FIG. 3A, thereby showing the inner view of the distal portion of FIG. 1 in accordance with embodiments described herein.

FIG. 3D depicts a perspective view of a C ring for use with an example device in accordance with an embodiment described herein.

FIG. 4 shows a front view of a distal portion of a device as described herein shown with a centering cage in a radially constrained configuration according to embodiments described herein.

FIG. 5 shows a front view of distal portion of a device as described herein shown with a centering cage in a radially expanded configuration according to embodiments described herein.

FIG. 6A depicts a perspective side view of the distal portion of a device in accordance with an embodiment described herein.

FIG. 6B depicts a perspective side view of the distal portion of a device in accordance with an embodiment described herein.

FIG. 6C depicts a cross-sectional view of the distal portion of the device of FIG. 6C in accordance with an embodiment described herein.

FIG. 7 shows a side view of the distal portion of an alternate device with a compressed centering feature in accordance with an embodiment described herein.

FIG. 8A shows a perspective view of a telescoping feature of the distal portion of a device in accordance with an embodiment described herein.

FIG. 9 shows a perspective view of a telescoping feature of the distal portion of a device with a balloon dilator in accordance with an embodiment described herein.

FIGS. 10A-10N show side and perspective views of example tip variations that can be used with devices described herein.

FIG. 11 shows a side view of an alternate centering cage on a device as described herein in accordance with an embodiment described herein.

FIGS. 12A-12C show perspective views (12A and 12C) and side views (12B, 12D, 12E) of alternate centering cages on the distal ends of devices as described herein in accordance with embodiments described herein.

FIG. 13 shows a perspective view of an alternate centering cage on the distal end of a device described herein in accordance with embodiments described herein.

FIG. 14 shows a perspective view of multiple centering cages on the distal end of a device described herein in accordance with embodiments described herein.

FIG. 15 shows a side view of an alternate centering cage on the distal end of a device described herein in accordance with embodiments described herein.

FIGS. 16A-16C show a perspective view, a magnified perspective view, and an inner side view, respectively, of an alternate centering cage on the distal end of a device described herein in accordance with embodiments described herein.

FIG. 17 shows a side view of an example balloon-assisted centering cage on the distal end of a device described herein in accordance with embodiments described herein.

FIGS. 18A-18B show relaxed and expanded, respectively, perspective views of a distal end of an atheroplasty device described herein in accordance with embodiments described herein.

FIG. 19 shows a perspective view of an example actuator of the atheroplasty device of FIGS. 18A-18B on the proximal end of a device described herein in accordance with embodiments described herein.

FIG. 20A shows a perspective view of a proximal end of a dual-lumen device as described herein in accordance with embodiments described herein.

FIG. 20B shows a perspective view of a distal end of a dual-lumen device as described herein in accordance with embodiments described herein.

FIG. 20C shows a side view of the inside of a dual-lumen device as described herein in accordance with embodiments described herein at the junction of two guidewires.

FIG. 20D shows a perspective top view of the inside of a dual-lumen device as described herein in accordance with embodiments described herein.

FIGS. 21A-21B show a side view and a magnified side view, respectively, of an embodiment of a microcatheter device as described herein.

FIG. 22 shows a perspective top view of the proximal end of an example embodiment of an embolic protection device as described herein.

FIG. 23 shows a side view of an example embodiment of a coiled tip guidewire for use in devices described herein.

FIG. 24 shows a top view of an embodiment of a steering stylet for deflection of tips of example devices described herein.

FIGS. 25A-25D show external side views (25A-25C) and a magnified internal perspective view (25D) of an example hybrid catheter for use with example devices and methods described herein.

FIGS. 26A-26K show side views of the use of a device as described herein for centering and crossing a vascular occlusion and advancing a guidewire in accordance with embodiments described herein.

FIGS. 27A-27D show side views of the use of an embolic protection device as described herein for capturing debris released from crossed occlusions in accordance with embodiments described herein.

FIGS. 28A-28C show side views of the use of a steering stylet in deflecting the tip from an original vessel to a desired vessel at a branching point in accordance with methods described herein.

FIGS. 29A-29B show side views of the use of a telescoping feature of an example telescoping device as described herein in accordance with methods described herein.

FIGS. 30A-30C show side views of an alternate use of a telescoping feature of an example telescoping device as described herein in accordance with methods described herein.

FIG. 31 shows a side view of a coronary stabilization method with a low loop in the aorta in accordance with methods described herein.

FIG. 32 shows a side view of a coronary stabilization method with a high loop in the aorta in accordance with methods described herein.

FIG. 33 shows a side view of a coronary stabilization method with a horizontal loop in the aorta in accordance with methods described herein.

FIGS. 34A-34B show a cross-sectional view and a side view, respectively, captured with intravascular ultrasound (IVUS) of a channel in the vessel resulting from passage of a device as described herein as it crossed through an occlusion using methods described herein.

FIGS. 35A-35B show a cross-sectional view and a side view, respectively, captured with intravascular ultrasound (IVUS) of a channel in the vessel resulting from passage of a device as described herein as it crossed through an occlusion using methods described herein.

FIG. 36A shows a side view, captured with an angiogram, of a channel in the vessel resulting from passage of a device as described herein as it crossed through an occlusion using methods described herein.

FIG. 36B illustrates a graph of the diameter and area of the channel shown in FIG. 32A resulting from passage of a device as described herein as it crossed through an occlusion using methods described herein.

FIGS. 37A-37B illustrate graphs of channel diameters resulting from passage of a device as described herein as it crossed through an occlusion using methods described herein and captured using an angiogram or an intravascular ultrasound (IVUS), respectively.

FIG. 38 illustrates a graph of the cross-sectional areas of channels resulting from passage of a device as described herein as it crossed through an occlusion using methods described herein and captured using an angiogram.

FIGS. 39A-39B illustrate graphs of channel diameters and cross-sectional areas, respectively, resulting from passage of a device through an occlusion as described herein as it crossed through an occlusion using methods described herein.

FIG. 40 shows arterial histology of use of example devices described herein.

FIG. 41 shows a side view of parts of an example cutting tip in accordance with example embodiments described herein.

FIGS. 42A-42C show front views of example setups for measuring tip load in accordance with embodiments described herein.

FIG. 43 shows a graph of tip loads and penetration powers of example guidewires compared to example devices as disclosed in embodiments described herein.

### DETAILED DESCRIPTION

Chronic total occlusions (CTO) are vascular occlusions that can block most or all blood flow through a blood vessel. CTOs can block 90% or more of blood flow. These occur due to plaque and/or occlusion mass buildup in the vessels, often arising from excess cholesterol deposits. While most occlusions are soft or of moderate hardness, a small percentage may be fully calcified. Approximately 60% can be soft, 25% can be moderately hard, and 15% can be highly calcified. CTOs can occur in most blood vessels, including coronary arteries, carotid arteries, iliac arteries and veins, femoral arteries and veins, and popliteal arteries and veins. Occlusions can also occur in veins (e.g., venous occlusions). In some cases, venous occlusions may be more fibrous and/or spongy than arterial occlusion, which may comprise more plaque and/or occlusion mass derived from cholesterol. CTO lesions may develop over the course of several months to years. Due to this chronic pathology, there can be an adequate amount of time for the development of collateral vessels to supply blood to tissue. These collateral vessels, however, can often fail to provide enough blood flow to keep organs alive and support their proper functioning, leading to chest pain, leg pain with walking or at rest, organ death, cardiac arrest, amputation, and even death.

Atherectomy, angioplasty, and bypass surgeries are methods developed to treat chronic total occlusions. However, there can be multiple undesirable side effects from these methods. Due to the sharpness of the device, the size of the device relative to the size of the vessel, and the difficulty in control, atherectomies may result in perforation or dissection of the vessel wall. Depending on the size and location of the vessel, for example vessels of the heart, this may necessitate immediate surgery and may have a high chance of death. Although angioplasty may not have the side effect of perforation, by pushing an expanded balloon through an occlusion, the vessel wall is expanded. This can lead to complications and side effects, for example dissection and tears, especially among those who need repeated procedures that further expand the vessel wall. Both procedures are lengthy and difficult, although not invasive. Both atherectomy and angioplasty can face difficulty with fully calcified plaque and/or occlusion masses. Bypass surgery is an invasive surgery that aims to bypass the occlusion instead of going through it. This is one approach that can be used for heavily calcified plaque and/or occlusion masses, which comprise approximately 15% of occlusions. These surgeries are frequently used for occlusions in vessels of the heart, but are lengthy, difficult, and expensive.

For these reasons, there is a need to provide improved devices and methods for crossing vascular occlusions or other blockages formed within blood vessels to treat the occlusion as well as to create pathways for the placement of guidewires, interventional devices and catheters. In particular, minimally-invasive devices for creating centered passages without high risk of damage to the vessel wall can be beneficial. Such devices may be relatively inexpensive to produce and relatively simple to use.

Provided herein are devices and methods for crossing occlusions. In some embodiments, the devices and methods described herein do not expand or stretch the vessel wall. The vessels can comprise coronary arteries, carotid arteries, iliac arteries and veins, femoral arteries and veins, pedal arteries, popliteal arteries, radial arteries, and other arteries and veins throughout the body. In some cases, the devices comprise a centering element that keeps them centered in the vessel, thereby significantly decreasing the chance of perforation or dissection of the vessel wall. In some cases, the devices are small, non-invasive devices that can be made relatively cheaply.

Disclosed herein are devices for tunneling through an occlusion. The devices can comprise a handle, a shaft, and a tip. In some cases, the shaft comprises a centering cage surrounding a shaft body. In some cases, there are multiple variations of centering cages depending on the occlusion to be tunneled through. In some cases, the tip comprises a cutting edge. The cutting edge can have multiple variations depending on the occlusion to be tunneled through. The tip can have multiple designs depending on the occlusion to be tunneled through. The labeling of devices with different numbers does not indicate that the devices are only usable with the features they are used to demonstrate. Rather, any device numbered or described herein can be used with any feature described herein.

Provided herein are methods for tunneling through an occlusion. In some cases, tunneling can comprise drilling, cutting, penetrating, pulverizing, blunt dissection or any combination thereof. In some cases, tunneling can comprise tunneling via a pushing force, via rotation, or via both. In some cases, the devices can be used to simultaneously exert a pushing force and a rotation. In some cases, simultaneously pushing and rotating can be more effective at tunneling than using one or the other method alone. In some cases, one or more devices described herein can be used to tunnel through an occlusion. A guidewire, catheter, or other device can then be extended past the occlusion. In some cases, one or more devices described herein can be used to tunnel partway through an occlusion, with the remainder of the occlusion being traversed by a guidewire. In some embodiments, one device as described herein can tunnel partway through the occlusion and can then be switched out for another device as described herein. This can be beneficial when a single occlusion has multiple properties, for example part of the occlusion is soft and part is moderately hard. In some cases, once one or more devices tunnel through the occlusion, the one or more devices can be removed and leave a tunnel in the occlusion. In some embodiments, the tunnel is formed by the dottering effect, wherein the occlusion mass can be compressed and pushed to the sides of the vessel wall (e.g., radially displaced). This way, a tunnel can be formed without cutting away and removing the occlusion mass as in an atherectomy. In some embodiments, the vessel wall is not stretched, extended, or expanded. Vessel dilation can be avoided by creating an opening that is only approximately from 30% to 70% of the available area of a clean vessel. This can be enough to restore blood flow without putting sufficient pressure on vessel walls that they dilate. In some cases, the vessel wall is not dissected, cut, perforated, or damaged. In some cases, an embolic protection device is used to capture debris released after crossing the occlusion. Any device described herein comprising any feature described herein can be used to tunnel through an occlusion.

Barotrauma, or pressure-based injury, to vessel walls can occur due to a high strain rate. As such, stretching the vessel wall slowly may not cause as much damage, if any. The devices described herein may have a strain rate of zero or one that is negligible to vessel walls in non-balloon variations due to lack of lateral force application and thus no active application of lateral strain rate. As such, there may be no vessel dilation or injury in those variations. For variations with a balloon, the pressure may be kept sufficient to dilate an occlusion but minimized to decrease effect on vessel walls.

Described herein are methods for inserting the devices described herein. In some cases, inserting can comprise insertion at a radial artery. In some cases, inserting can comprise insertion at a pedal artery. In some cases, insertion can comprise insertion at a brachial, femoral, or popliteal artery. In some embodiments, inserting can comprise extending the device to an occlusion mass of interest via a guiding catheter. In some cases, inserting can comprise steering through branching and/or tortuous vessels.

In some cases, the devices used vary depending on the location of insertion. In some cases, the device features vary depending on the location of insertion. For example, if a small device is needed for a small vessel but the occlusion is of moderate hardness, a sharper tip and cutter can be used in conjunction with the narrower device.

In some cases, the devices and methods described herein may use one or more different mechanisms to disrupt an occlusion mass. In some cases, the devices and methods described herein may use one or more of mechanical, vibrational, ultrasound, thermal, cooling, cryo, or optical energy to disrupt the occlusion mass. Mechanical disruption can include pushing, cutting, drilling, or other methods described herein. Vibrational disruption may include a vibration device nestled within the handle or the distal end of the devices described herein. Ultrasound disruption may include an ultrasound transducer nestled within the distal end of the devices described herein, for example in the tip, such that ultrasound energy is directed into an occlusion mass. There may be an internal imaging device within the distal end of the device in addition to or alternatively to an ultrasound energy applier. The imaging device can be a camera, an ultrasound, or any other type of imaging device. Thermal energy can include heating, cooling, and cryo-based energy. Thermal energy can be used to make the occlusion mass malleable or brittle. Thermal energy may be used with mechanical disruption after making the occlusion mass malleable or brittle. Optical energy may include light energy generated in the handle, shown into the handle, generated in the shaft, generated in the distal end, or generated in the tip. In some cases, the light sources may be in the handle, shaft, distal end, or tip. In some cases, there may be reflective or refractive mirrors in the handle, shaft, distal end, or tip. In some cases, for example when the light source or mirror is located in the inner shaft or tip, rotating the inner shaft or tip may rotate the light source or mirror, thus creating a “light drilling” effect.

In some cases, the devices and methods disclosed herein can be used for crossing chronic total occlusions. This can include creating channels in the occlusions or enlarging small channels that have zero or negligible blood flow. In some cases, the devices and methods disclosed herein can be used for crossing partial occlusions. This can include enlarging channels that provide little or insufficient blood flow to a given anatomical area.

There are multiple terms throughout the applications that may be used to refer to various occlusions. In some cases, the most common terms may be “occlusion,” “plaque and/or occlusion mass,” and “occlusion mass.” In some cases, “occlusion” and “plaque and/or occlusion mass” may refer to arterial blockages, whereas “occlusion mass” is a broader term that may include venous occlusions because of the frequently fibrous nature of venous occlusions.

### Devices

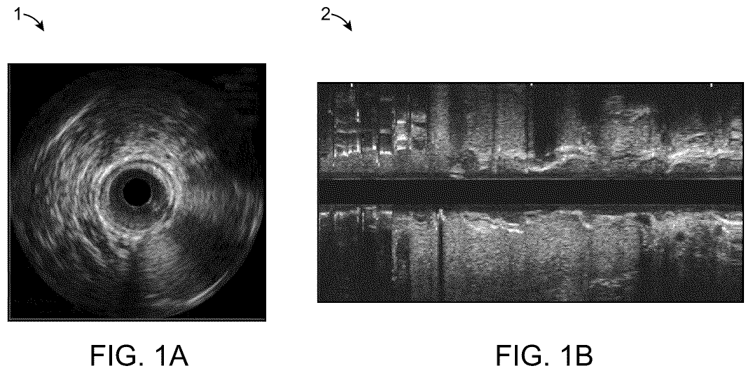

Disclosed herein are devices for crossing occlusions. In some cases, the devices comprise a self-centering system that keeps them centered within the vessel during the intraluminal crossing. In some cases, the devices tunnel through the occlusions through drilling, cutting, penetrating, pulverizing, blunt dissection or any combination thereof of the plaque and/or occlusion mass. In some cases, the drilling, cutting, penetrating, pulverizing, blunt dissection or any combination thereof is accomplished by a cutting loop. In some cases, the cutting loop penetrates the occlusion and rotates. In some cases, one or more of the tip and the centering system of the device radially displaces the plaque and/or occlusion mass. In some cases, one or more of the tip and the centering system compresses the plaque and/or occlusion mass against the vessel wall adjoining the tip and centering system along the length between the tip and centering system. In some cases, as shown in FIGS. 1A-1B, the devices create an intraluminal wide tract formation while crossing the occlusion, which can be seen once the device is removed.

In some cases, the devices disclosed herein may have a high tip load. Tip load can be measured when a wire is held 10 mm from a load cell and pressed against the load cell. The load cell amount under which the wire buckles can be the tip load. In some cases, for example when the device consists of more than the wire as in the devices described herein, tip load can be used to described a pushing strength of a device. Tip load can be used to describe the pushing and puncturing strength of the devices described herein as a combination of their cutting tips, guide wire, and overall device structure. In some cases, tip load is measured in grams.

The tip load can reflect the puncture strength of the guidewire when held 10 mm from the tip.

However, during conventional usage in vascular interventions, the guidewire can be held anywhere from about 50 cm to about 150 cm from the tip, hence the “effective tip load” in a clinical setting is far less than the tip load measured in the standard lab setting when the guidewire is held at 10 mm from the tip. For example, basic guidewire tip loads may be 40 g but only have about 20 g reach the occlusion mass during routine vascular intervention (effective tip load), as they are held far away from its tip. Depending on the softness or hardness of occlusions, the effective tip load to penetrate the occlusion may vary.

FIG. 41 shows the tip of a cutting loop 4100 of a device as described herein. The tip of the cutting loop can be separated into the forward cutting surface 4102 and the lateral plaque displacing surface 4104. Both 4102 and 4104 can add to the tip load of the device. Forward cutting surface 4102 also determines the penetration power of the device. The tip load and penetration power can depend on how the cutting loop interacts with the occlusion. As such, the tip load and penetration power of the devices with two cutting loops may vary depending on the angle with which the cutting loops interact with the occlusion mass. For example, the device may interact with the occlusion mass straight-on, such that both cutting loops contact the mass at substantially the same time. In some cases, the device may interact with the occlusion mass at an angle, such that one loop contacts the mass before the other.

As discussed below in the examples, the tip load of the devices disclosed herein can be between about 11.6 times larger (comparing the lowest tip load in the thinner cardiovascular devices disclosed herein against the largest tip load of the third-party devices) to about 2,226 times larger (comparing the highest tip load in the thicker peripheral devices disclosed herein against the smallest tip load of the third-party devices). Similarly, the penetration power was between about 1.2 times larger (comparing the lowest tip load in the cardiovascular devices disclosed herein against the largest tip load of the third-party devices) to about 210 times larger (comparing the highest tip load in the peripheral devices disclosed herein against the smallest tip load of the third-party devices). The coronary device tip load can be smaller than the peripheral vessel tip load. The larger penetration powers and tip loads in the cardiovascular and peripheral devices disclosed herein can allow for penetration of hard, calcified plaques that third-party devices may not penetrate.

Tip loads of guidewires may be between about 1 g to about 40 g. However, in some cases, the tip loads of the devices described herein may be 1300 g in large blood vessels with larger devices, 200 g in small blood vessels with smaller devices, along with in that range, higher, and lower depending on the specific device of the versions disclosed herein. In some cases, the tip loads of the devices described herein may be from about 50 g to about 1500 g. In some cases, the tip loads of the devices described herein may be from about 50 g to about 100 g, about 50 g to about 150 g, about 50 g to about 200 g, about 50 g to about 250 g, about 50 g to about 300 g, about 50 g to about 350 g, about 50 g to about 400 g, about 50 g to about 450 g, about 50 g to about 500 g, about 100 g to about 150 g, about 100 g to about 200 g, about 100 g to about 250 g, about 100 g to about 300 g, about 100 g to about 350 g, about 100 g to about 400 g, about 100 g to about 450 g, about 100 g to about 500 g, about 150 g to about 200 g, about 150 g to about 250 g, about 150 g to about 300 g, about 150 g to about 350 g, about 150 g to about 400 g, about 150 g to about 450 g, about 150 g to about 500 g, about 200 g to about 250 g, about 200 g to about 300 g, about 200 g to about 350 g, about 200 g to about 400 g, about 200 g to about 450 g, about 200 g to about 500 g, about 250 g to about 300 g, about 250 g to about 350 g, about 250 g to about 400 g, about 250 g to about 450 g, about 250 g to about 500 g, about 300 g to about 350 g, about 300 g to about 400 g, about 300 g to about 450 g, about 300 g to about 500 g, about 350 g to about 400 g, about 350 g to about 450 g, about 350 g to about 500 g, about 400 g to about 450 g, about 400 g to about 500 g, or from about 450 g to about 500 g. In some cases, the tip loads of the devices described herein may be from about 1000 g to about 1100 g, about 1000 g to about 1200 g, about 1000 g to about 1300 g, about 1000 g to about 1400 g, about 1000 g to about 1500 g, about 1100 g to about 1200 g, about 1100 g to about 1300 g, about 1100 g to about 1400 g, about 1100 g to about 1500 g, about 1200 g to about 1300 g, about 1200 g to about 1400 g, about 1200 g to about 1500 g, about 1300 g to about 1400 g, about 1300 g to about 1500 g, or from about 1400 g to about 1500 g. In some cases, the tip loads of the devices described herein may be from about 50 g to about 250 g, about 50 g to about 450 g, about 50 g to about 650 g, about 50 g to about 850 g, about 50 g to about 1050 g, about 50 g to about 1250 g, about 50 g to about 1400 g, about 50 g to about 1500 g, about 250 g to about 450 g, about 250 g to about 650 g, about 250 g to about 850 g, about 250 g to about 1050 g, about 250 g to about 1250 g, about 250 g to about 1400 g, about 250 g to about 1500 g, about 450 g to about 650 g, about 450 g to about 850 g, about 450 g to about 1050 g, about 450 g to about 1250 g, about 450 g to about 1400 g, about 450 g to about 1500 g, about 650 g to about 850 g, about 650 g to about 1050 g, about 650 g to about 1250 g, about 650 g to about 1400 g, about 650 g to about 1500 g, about 850 g to about 1050 g, about 850 g to about 1250 g, about 850 g to about 1400 g, about 850 g to about 1500 g, about 1050 g to about 1250 g, about 1050 g to about 1400 g, about 1050 g to about 1500 g, about 1250 g to about 1400 g, about 1250 g to about 1500 g, or from about 1400 g to about 1500 g.

In some cases, the tip loads of the devices described herein may be less than about 50 g, less than about 100 g, less than about 150 g, less than about 200 g, less than about 250 g, less than about 300 g, less than about 350 g, less than about 400 g, less than about 450 g, less than about 500 g, less than about 550 g, less than about 600 g, less than about 650 g, less than about 700 g, less than about 750 g, less than about 800 g, less than about 850 g, less than about 900 g, less than about 950 g, less than about 1000 g, less than about 1050 g, less than about 1100 g, less than about 1150 g, less than about 1200 g, less than about 1250 g, less than about 1300 g, less than about 1350 g, less than about 1400 g, less than about 1450 g, or less than about 1500 g. In some cases, the tip loads of the devices described herein may be greater than about 50 g, greater than about 100 g, greater than about 150 g, greater than about 200 g, greater than about 250 g, greater than about 300 g, greater than about 350 g, greater than about 400 g, greater than about 450 g, greater than about 500 g, greater than about 550 g, greater than about 600 g, greater than about 650 g, greater than about 700 g, greater than about 750 g, greater than about 800 g, greater than about 850 g, greater than about 900 g, greater than about 950 g, greater than about 1000 g, greater than about 1050 g, greater than about 1100 g, greater than about 1150 g, greater than about 1200 g, greater than about 1250 g, greater than about 1300 g, greater than about 1350 g, greater than about 1400 g, greater than about 1450 g, or greater than about 1500 g. In some cases, the tip loads of the devices described herein may be greater than about 1500 g, about 1600 g, 1700 g, 1800 g, 1900 g, 2000 g, 2100 g, 2200 g, or greater than about 2300 g.

The tip load delivered to the occlusion may be between about 1/10 to about ⅕ of the original tip load. The tip load delivered to the occlusion masses may be from about 200 g to about 300 g. In some cases, the tip load delivered to the occlusion masses may be from about 100 g to about 400 g. In some cases, the tip load delivered to the occlusion masses may be from about 100 g to about 150 g, about 100 g to about 200 g, about 100 g to about 250 g, about 100 g to about 300 g, about 100 g to about 350 g, about 100 g to about 400 g, about 150 g to about 200 g, about 150 g to about 250 g, about 150 g to about 300 g, about 150 g to about 350 g, about 150 g to about 400 g, about 200 g to about 250 g, about 200 g to about 300 g, about 200 g to about 350 g, about 200 g to about 400 g, about 250 g to about 300 g, about 250 g to about 350 g, about 250 g to about 400 g, about 300 g to about 350 g, about 300 g to about 400 g, or from about 350 g to about 400 g. In some cases, the tip load delivered to the occlusion masses may be less than about 100 g, less than about 150 g, less than about 200 g, less than about 250 g, less than about 300 g, less than about 350 g, or less than about 400 g. In some cases, the tip load delivered to the occlusion masses may be greater than about 100 g, greater than about 150 g, greater than about 200 g, greater than about 250 g, greater than about 300 g, greater than about 350 g, or greater than about 400 g.

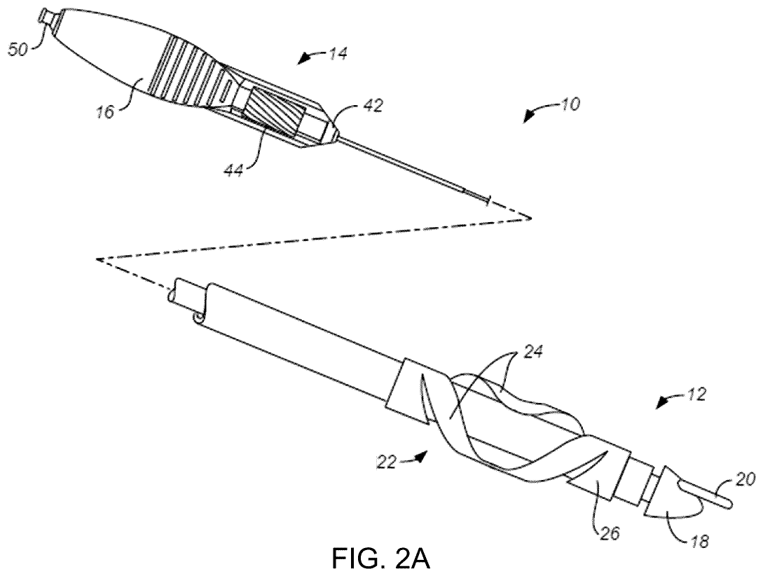

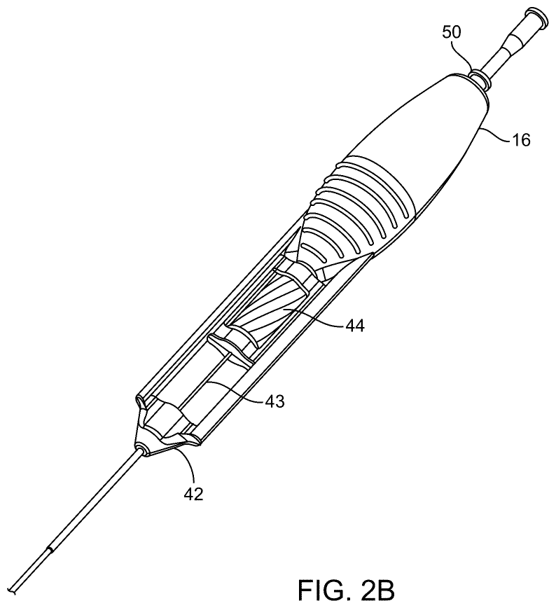

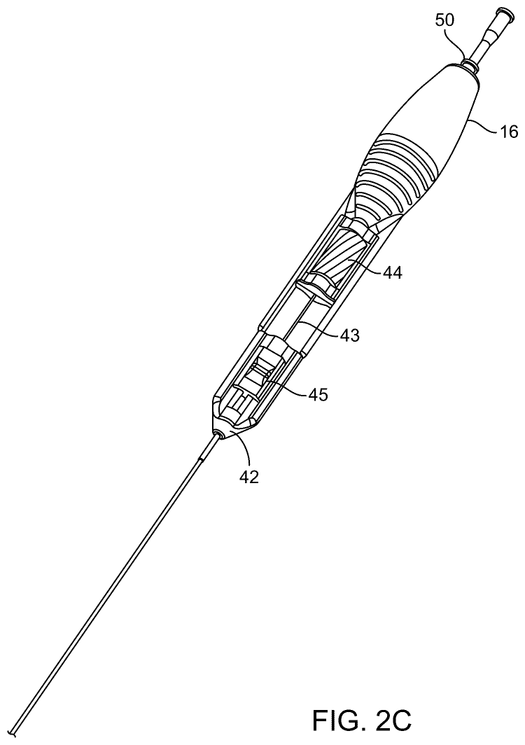

FIG. 2A illustrates a first embodiment of a device 10 constructed in accordance with the embodiments described herein. A device 10 for centering and crossing vascular occlusions can comprise a device body comprising a distal end 12 and a proximal end 14 comprising a handle 16. In some cases, device 10 is a catheter. A rotatable cutting tip 18 can be located at a distal tip of the distal end 12 and can comprise a single cutting loop 20 which may be rotated as described in more detail below. The cutting tip 18 can be rotated by rotating a wheel on a handle. A user can determine the direction (e.g., clockwise or counterclockwise) and the speed of the rotation based on the rotation of handle 16. In some cases, the speed of rotation can be from about 30 RPM to about 60 RPM. The speed can be less than about 30 RPM or greater than about 60 RPM. The cutting tip 18 can be rotated at varying speeds. The cutting tip 18 and cutting loop 20 can have multiple variations, as described below, depending on the size, location, and density of the occlusion to tunnel through.

A centering cage 22 can circumscribe the distal end 12 to maintain centering of the distal end 12 within a vascular lumen as the device 10 is advanced therethrough. In particular, the centering cage 22 can comprise a plurality of planar, spiral springs 24. In some cases, the centering cage comprises three spiral springs 24. In some cases, the springs are planar but not spiral. The spiral springs 24 can be formed by cutting or otherwise patterning a cylindrical tube 26, where the entire tube may then be secured to the distal end 12 of the device 10. The cylindrical tube 26 can be formed from nitinol (NiTi) or other shape memory metals/alloys and may be heat-set so that the individual spiral springs 24 are in their radially expanded configuration in the absence of a radial constraint, such as a delivery sheath, guide catheter, or the like.

The handle 16 can be ribbed, as shown in FIGS. 2A-2C. This can improve traction and keep the handle in the user's hand. The handle 16 can be smooth. The handle 16 can comprise a nose 42 located on the distal end of the proximal end 14 of device 10. The handle can comprise a wheel or spindle 44 which is rotatably mounted in the handle 16 to permit manual rotation of the wheel or spindle relative to the handle. A user can rotate the device by rotating the wheel or spindle 44 on handle 16. A user can determine the direction (e.g., clockwise or counterclockwise) and the speed of the rotation based on the rotation of handle 16. In some cases, the speed of rotation can be from about 30 RPM to about 60 RPM. The speed can be less than about 30 RPM or greater than about 60 RPM. In some cases, there may be a button, lever, sliding lever, or other type of control to automatically, electronically, or mechanically rotate the wheel 44 without the user physically rotating the handle. The outer shaft is fixedly attached so that the outer will be prevented from rotating relative to the handle. In contrast, the inner shaft is attached to the wheel or spindle 44. Rotation of the wheel or spindle 44, in turn, will rotate the inner shaft which and in turn rotates the tip 18 and cutting loop 20. In some cases, the wheel or spindle 44 may be disposed on a shuttle that can slide forward, backwards, or both along the distal end of the handle. The wheel or spindle 44, through the shuttle, may slide in one or both directions by less than about 0.2 inches, less than about 0.4 inches, less than about 0.6 inches, less than about 0.8 inches, or less than about 1 inch. The wheel or spindle 44, through the shuttle, may slide in one or both directions by greater than about 0 inches, greater than about 0.2 inches, greater than about 0.4 inches, greater than about 0.6 inches, greater than about 0.8 inches, or greater than about 1 inch. The wheel or spindle 44, through the shuttle, may slide in one or both directions by from about 0 inches to 1 inch. The wheel or spindle 44, through the shuttle, may slide in one or both directions by from about 0 inches to about 0.2 inches, about 0 inches to about 0.4 inches, about 0 inches to about 0.6 inches, about 0 inches to about 0.8 inches, about 0 inches to about 1 inch, about 0.2 inches to about 0.4 inches, about 0.2 inches to about 0.6 inches, about 0.2 inches to about 0.8 inches, about 0.2 inches to about 1 inch, about 0.4 inches to about 0.6 inches, about 0.4 inches to about 0.8 inches, about 0.4 inches to about 1 inch, about 0.6 inches to about 0.8 inches, about 0.6 inches to about 1 inch, or from about 0.8 inches to about 1 inch.

The ability to slide the wheel 44 attached to the inner shaft and, through the inner shaft, the tip, can allow a user to impart forward force. Sliding the wheel 44 can provide force control, or at-will force, to the user to proceed through an occlusion with this device. In conjunction with the flexibility in some embodiments disclosed below, some embodiments of the device may provide both enhanced strength and flexibility for both maneuvering in branching or bending vasculature while retaining ability to cut through an occlusion.

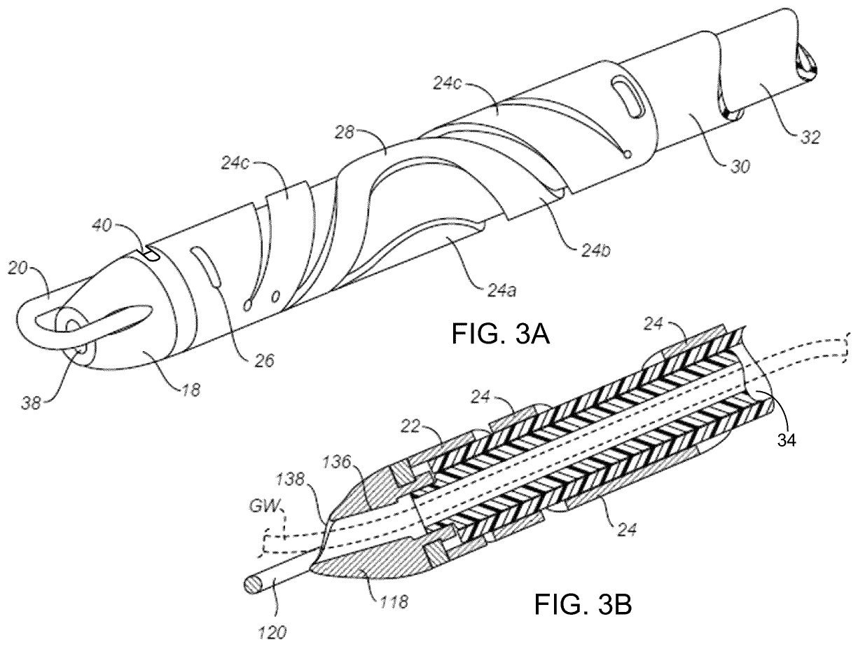

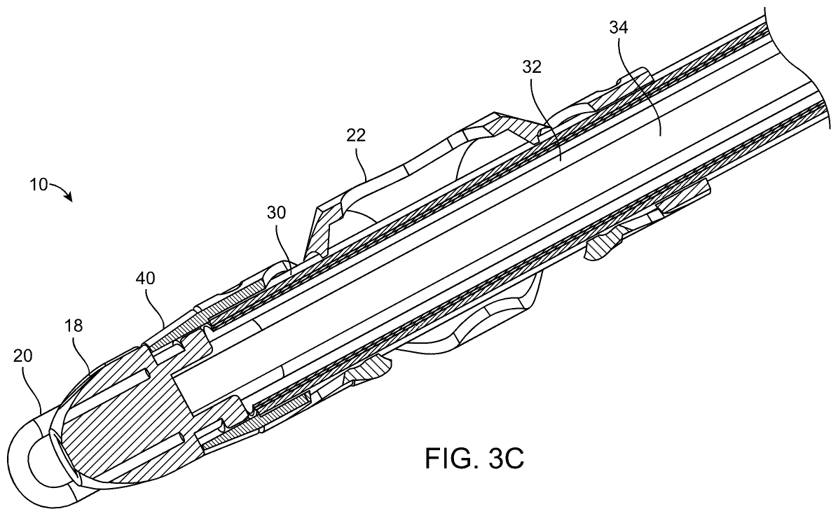

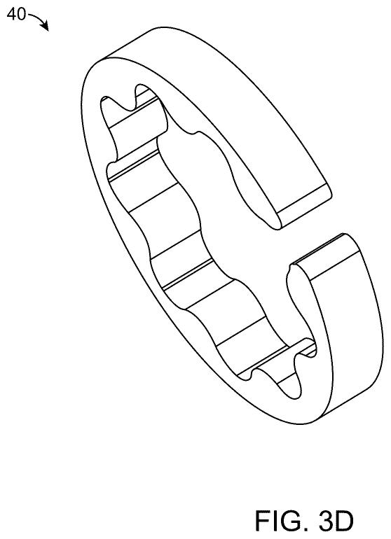

The proximal end 14 of device 10 comprises a luer 50 on the proximal end of the handle 16. The luer can be rotationally fixed within the handle. Guidewires and other interventional elements may be introduced through the luer 50 so that they may be advanced to the distal tip and lumen of the inner shaft of the device 10 and out through a distal opening 38 of the cutting tip 18 as shown in FIG. 3B.

The device 10 can be employed above the knee and below the knee in an antegrade or retrograde direction. The device 10 can be inserted at a radial vessel for access to peripheral vessels in an antegrade direction. The device 10 can be manufactured in smaller and larger sizes depending on the size of the vessel, wherein smaller and larger denote the diameter of the device as measured at the cylindrical tube 26. In some cases, the device 10 can be smaller to be inserted at a radial vessel or another peripheral vessel. In some cases, the device 10 can be larger to be employed above or below the knee.

In some embodiments, the diameter of the device 10 as measured at the cylindrical tube 26 can be from about 1 mm and about 4 mm. The diameter can be from about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 2 mm to about 2.5 mm, about 2 mm to about 3 mm, about 2 mm to about 3.5 mm, about 2 mm to about 4 mm, about 2.5 mm to about 3 mm, about 2.5 mm to about 3.5 mm, about 2.5 mm to about 4 mm, about 3 mm to about 3.5 mm, or from about 3 mm to about 4 mm. In some cases, the diameter is less than about 1 mm, less than about 1.5 mm, less than about 2 mm, less than about 2.5 mm, less than about 3 mm, less than about 3.5 mm, or less than about 4 mm. In some cases, the diameter is greater than about 1 mm, greater than about 1.5 mm, greater than about 2 mm, greater than about 2.5 mm, greater than about 3 mm, greater than about 3.5 mm, or greater than about 4 mm. In some cases, the diameter can be from about 1.6 mm and 2.3 mm, or from about 5 Fr to about 7 Fr. In some cases, the diameter is about 2 mm, about 2.5 mm, or about 3.5 mm.

In some cases, the crossing profile of the device may be from about 2.5 Fr to about 11 Fr. In some cases, the crossing profile of the device may be from about 2.5 Fr to about 5 Fr, about 2.5 Fr to about 7 Fr, about 2.5 Fr to about 9 Fr, about 2.5 Fr to about 11 Fr, about 5 Fr to about 7 Fr, about 5 Fr to about 9 Fr, about 5 Fr to about 11 Fr, about 7 Fr to about 9 Fr, about 7 Fr to about 11 Fr, or about 9 Fr to about 11 Fr. In some cases, the crossing profile of the device may be about 2.5 Fr, about 5 Fr, about 7 Fr, about 9 Fr, or about 11 Fr. In some cases, the crossing profile of the device may be at least about 2.5 Fr, about 5 Fr, about 7 Fr, or about 9 Fr. In some cases, the crossing profile of the device may be at most about 5 Fr, about 7 Fr, about 9 Fr, or about 11 Fr.

In some embodiments, the diameter of the tip 18 can be from about 1 mm and about 1.8 mm. In some embodiments, the diameter of the tip 18 can be from about 1 mm to about 1.2 mm, about 1 mm to about 1.4 mm, about 1 mm to about 1.6 mm, about 1 mm to about 1.8 mm, about 1.2 mm to about 1.4 mm, about 1.2 mm to about 1.6 mm, about 1.2 mm to about 1.8 mm, about 1.4 mm to about 1.6 mm, about 1.4 mm to about 1.8 mm, or from about 1.6 mm to about 1.8 mm. In some cases, the diameter can be less than 1 mm, less than 1.2 mm, less than 1.4 mm, less than 1.6 mm, or less than 1.8 mm. In some cases, the diameter can be greater than 1 mm, greater than 1.2 mm, greater than 1.4 mm, greater than 1.6 mm, or greater than 1.8 mm. In some cases, the diameter can be from about 1.1 mm to about 1.35 mm. In some cases, the diameter can be from about 1.1 mm to about 1.15 mm, about 1.1 mm to about 1.2 mm, about 1.1 mm to about 1.25 mm, about 1.1 mm to about 1.3 mm, about 1.1 mm to about 1.35 mm, about 1.15 mm to about 1.2 mm, about 1.15 mm to about 1.25 mm, about 1.15 mm to about 1.3 mm, about 1.15 mm to about 1.35 mm, about 1.2 mm to about 1.25 mm, about 1.2 mm to about 1.3 mm, about 1.2 mm to about 1.35 mm, about 1.25 mm to about 1.3 mm, about 1.25 mm to about 1.35 mm, or from about 1.3 mm to about 1.35 mm.

In some cases, the diameter can be about 1.6 mm. In some cases, the device 10 for antegrade use above the knee can have a diameter of about 3.5 mm and a tip diameter of about 1.6 mm.

In some cases, the device 10 for antegrade use below the knee can have a diameter of about 2.5 mm and a tip diameter of about 1.35 mm. In some cases, the device 10 for antegrade use when inserted into a radial vessel can have a diameter of about 2 mm to about 2.5 mm and a tip diameter of about 1.1 mm to about 1.35 mm.

The device 10 can comprise a working length calculated from the handle 16 to the cutting loop 20 and including an extension of distal end 12 from proximal end 14. In some cases, the working length can be from about 100 cm and about 200 cm. In some cases, the working length can be from about 100 cm to about 120 cm, about 100 cm to about 140 cm, about 100 cm to about 160 cm, about 100 cm to about 180 cm, about 100 cm to about 200 cm, about 120 cm to about 140 cm, about 120 cm to about 160 cm, about 120 cm to about 180 cm, about 120 cm to about 200 cm, about 140 cm to about 160 cm, about 140 cm to about 180 cm, about 140 cm to about 200 cm, about 160 cm to about 180 cm, about 160 cm to about 200 cm, or from about 180 cm to about 200 cm. In some cases, the working length is less than about 100 cm, less than about 120 cm, less than about 140 cm, less than about 160 cm, less than about 180 cm, or less than about 200 cm. In some cases, the working length is greater than about 100 cm, greater than about 120 cm, greater than about 140 cm, greater than about 160 cm, greater than about 180 cm, or greater than about 200 cm.

In some cases, the device 10 for antegrade use above the knee can have a working length of about 125 cm. In some cases, the device 10 for antegrade use below the knee can have a working length of about 135 cm. In some cases, the device 10 for antegrade use when inserted into a radial vessel can have a working length of from about 160 cm to about 190 cm.

FIG. 2B shows an alternate handle or proximal end of a device 10 or other devices described herein. The handle 16 can comprise nose 42, luer 50, wheel 44, and track 43. A guidewire can proceed out of the nose 42. Guidewires and other interventional elements may be introduced through the luer 50 so that they may be advanced to the distal tip and lumen of the inner shaft of the device 10 and out through a distal opening 38 of the cutting tip 18 as shown in FIG. 3B. The wheel may function similarly to the wheel in FIG. 2A. The track 43 allows for forward and/or backward motion of the wheel to create a force as needed. The ability to slide the wheel 44 attached to the inner shaft and, through the inner shaft, the tip, can allow a user to impart forward force. Sliding the wheel 44 can provide force control, or at-will force, to the user to proceed through an occlusion with this device. In conjunction with the flexibility in some embodiments disclosed below, some embodiments of the device may provide both enhanced strength and flexibility for both maneuvering in branching or bending vasculature while retaining ability to cut through an occlusion.

The track 43 can have a length of from about 2 mm to about 20 mm. The track 43 can have a length of from about 2 mm to about 6 mm, about 2 mm to about 10 mm, about 2 mm to about 14 mm, about 2 mm to about 18 mm, about 2 mm to about 20 mm, about 6 mm to about 10 mm, about 6 mm to about 14 mm, about 6 mm to about 18 mm, about 6 mm to about 20 mm, about 10 mm to about 14 mm, about 10 mm to about 18 mm, about 10 mm to about 20 mm, about 14 mm to about 18 mm, about 14 mm to about 20 mm, about 18 mm to about 20 mm. The track 43 can have a length of less than about 2 mm, less than about 6 mm, less than about 10 mm, less than about 14 mm, less than about 18 mm, or less than about 20 mm. The track 43 can have a length of greater than about 2 mm, greater than about 6 mm, greater than about 10 mm, greater than about 14 mm, greater than about 18 mm, or greater than about 20 mm.

FIG. 2C shows an alternate handle or proximal end of a device 10 or other devices described herein. The handle 16 can comprise nose 42, luer 50, wheel 44, track 43, and handle tab 45. A guidewire can proceed out of the nose 42. Guidewires and other interventional elements may be introduced through the luer 50 so that they may be advanced to the distal tip and lumen of the inner shaft of the device 10 and out through a distal opening 38 of the cutting tip 18 as shown in FIG. 3B. The wheel may function similarly to the wheel in FIG. 2A. The track 43 allows for forward and/or backward motion of the wheel to create a force as needed. The ability to slide the wheel 44 attached to the inner shaft and, through the inner shaft, the tip, can allow a user to impart forward force. Sliding the wheel 44 can provide force control, or at-will force, to the user to proceed through an occlusion with this device. In conjunction with the flexibility in some embodiments disclosed below, some embodiments of the device may provide both enhanced strength and flexibility for both maneuvering in branching or bending vasculature while retaining ability to cut through an occlusion.

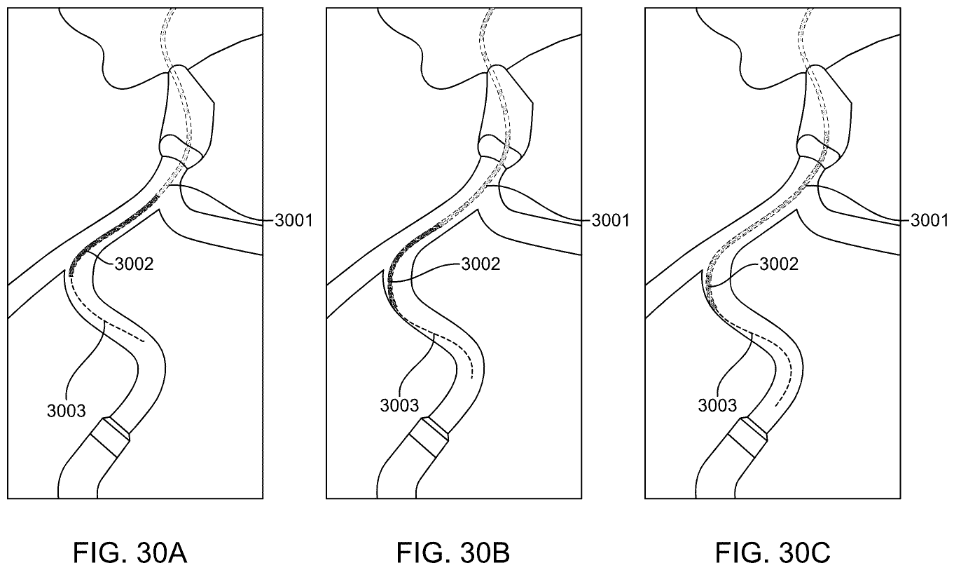

This variation of handle 16 also has the handle tab 45, which may be attached, connected to, or coupled to the outer shaft, rather than the inner shaft of the device. Handle tab 45 may also slide forward and/or backward along the track 43. The handle tab 45 and wheel 44 can be mobile independently. The addition of the handle tab 45 to the design of FIG. 2B can allow for additional strategies to navigate bending and branching vasculature. For example, as seen in FIGS. 30A-30C, described further below, the outer shaft can catch up to the inner shaft and may avoid maintaining a constant telescoping distance.

The track 43 can have a length of from about 2 mm to about 20 mm. The track 43 can have a length of from about 2 mm to about 6 mm, about 2 mm to about 10 mm, about 2 mm to about 14 mm, about 2 mm to about 18 mm, about 2 mm to about 20 mm, about 6 mm to about 10 mm, about 6 mm to about 14 mm, about 6 mm to about 18 mm, about 6 mm to about 20 mm, about 10 mm to about 14 mm, about 10 mm to about 18 mm, about 10 mm to about 20 mm, about 14 mm to about 18 mm, about 14 mm to about 20 mm, about 18 mm to about 20 mm. The track 43 can have a length of less than about 2 mm, less than about 6 mm, less than about 10 mm, less than about 14 mm, less than about 18 mm, or less than about 20 mm. The track 43 can have a length of greater than about 2 mm, greater than about 6 mm, greater than about 10 mm, greater than about 14 mm, greater than about 18 mm, or greater than about 20 mm.

FIG. 3A depicts a perspective side view of the distal portion of the device 10 of FIG. 1 shown with a centering cage in accordance with an embodiment described herein. FIGS. 3B-3C illustrate an inner and cross-sectional view, respectively, of the device 10 of FIG. 3A, thereby showing the inner view of the distal portion of FIG. 1 in accordance with embodiments described herein. The device 10 can comprise a cutting loop 20, a tip 18, an exit opening 38, a retaining ring 40, a centering cage 22, spirals 24, cylindrical tube 26, flat surface 28, outer shaft 30, inner shaft 32, and lumen 34. In FIG. 3B, some of these can correspond to 120, 118, and 138 as well as showing central passage 136. The centering cage 22 can comprise one or more flat, spiral springs 24. The centering cage 22 can comprise three springs 24. When the centering cage 22 comprises three springs 24, the springs can be labeled 24a-24c as shown in FIG. 3A. The three springs refer to the inner spring 24a, the middle spring 24b, and the outer spring 24c. In some cases, the flat surface 28 refers to a circular or elliptical radius created by the springs 24 in their expanded form, as discussed in further detail below.

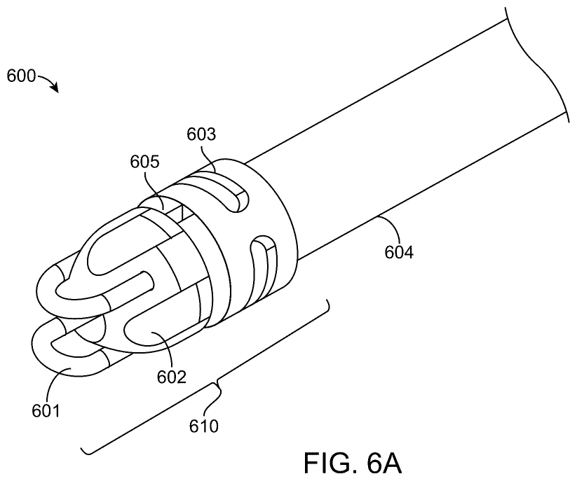

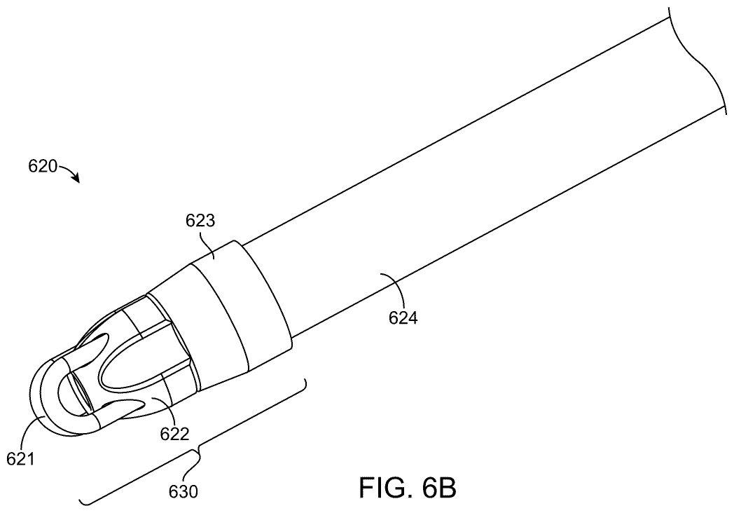

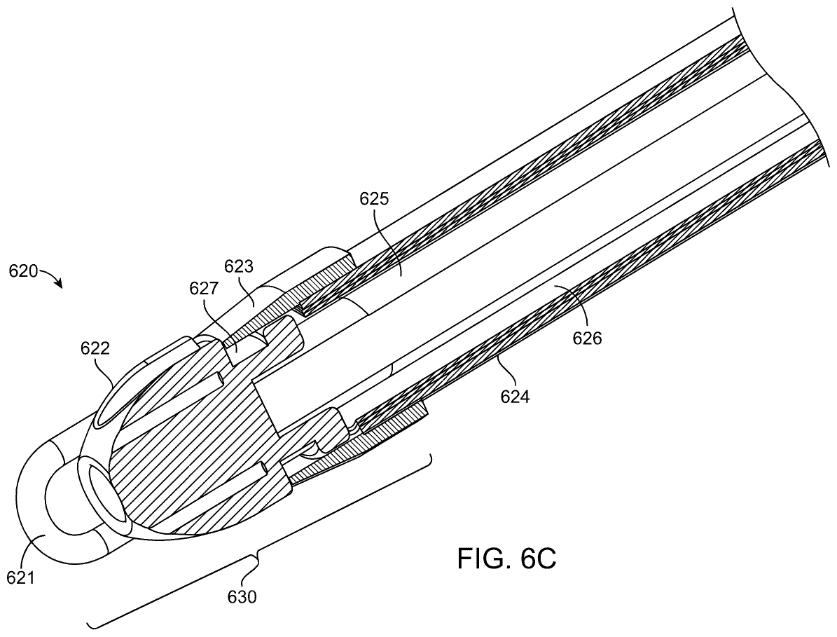

FIG. 3D shows C ring 40 (retainer ring) for use with a device 10, or other device as described herein. The C ring 40 may surround the inner shaft and may be located in the hole 627 (shown in FIG. 6C and similarly located in FIGS. 3A and 3C) between the OM ring and the inner shaft. The C ring 40 may have a smoothly rounded exterior. The C ring 40 may have a wavy interior. In some cases, the exterior may be rounded to fit under/between the OM ring and the tip. As discussed above, the C ring 40 may be bonded to the OM ring (outer member ring). This can allow the C ring 40 to remain in place while the inner shaft and tip rotate. In some cases, the interior may be wavy. This can decrease the contact between the C ring 40 and the inner shaft, such that the points of contact are mostly on the parts of the “waves” that are closest to the interior. By decreasing the contact between the C ring 40 and the inner shaft, the inner shaft can retain its ability to rotate.

It should be noted that there may be at least one or two possible types of rings in the devices described herein. In some cases, the devices described herein may comprise a “C ring” or a “retaining ring,” as described above with the functions described above. These two names may be synonymous because “C ring” can describe the shape while “retaining ring” can describe the functionality. In some cases, the devices described herein may comprise an “outer member,” “outer shaft,” or “OM” ring. This second type of ring may encircle the outer shaft. It can attach the retainer ring to the outer shaft or, in some embodiments described herein, limit the distal advancement of the cutting tip beyond the distal end of the device shaft. In some cases, the OM ring can be replaced by a centering cage. In some cases, the device may have either an OM ring or a centering cage. In some cases, the centering cage may function like an OM ring to allow the outer shaft to remain near the tip. In some cases, for example in devices without centering cages such as devices 600 and 620, the OM rings keep the outer shaft attached to or near the tip. The bonding of the outer shaft to the OM ring and in turn to the C ring may allow the outer shaft to stay static when the inner shaft and tip bonded to it are rotated by the wheel movement of the handle by the operator. The tip's neck (the narrower proximal end of the tip) can be located and rotate freely inside the C-ring's central wavy edge.

The device 10 can comprise an outer shaft 30 and an inner shaft 32. In some embodiments, the inner shaft 32 is concentrically disposed within a luminal passage of the outer shaft and is rotatable therein as will be described in more detail below. In some embodiments, the inner shaft 32 has a lumen 34 which is configured to accommodate a guidewire GW as well as for other purposes, such as infusion, suction, and the advancement of other interventional tools.

In some embodiments, the cutting tip 18 has a central passage which is aligned and contiguous with the lumen 34 of the inner shaft. In some embodiments, the central passage has a distal opening 38 through which a guidewire or other element, tool, or component may be advanced. In some cases, the cutting tip 18 is fixedly attached to a distal end of the inner shaft 32 so that rotation of the inner shaft will cause rotation of the cutting tip 18 as well as the cutting loop 20. Retaining ring 40 may be used to prevent the cutting tip and loop(s) from moving beyond the ring.

While the central passage of the cutting tip 18 may be axially aligned with the lumen 34 of the inner shaft as in FIG. 3A, in some embodiments, such as those intended for peripheral use as shown in FIG. 3B, a cutting tip 118 may have a centrally aligned cutting loop 120 which can have a passage 136. Passage 136 can be inclined or offset relative to its longitudinal axis so that the distal opening 138 deflects the guidewire away from the cutting loop 120.

The device 10 can be employed above the knee and below the knee in an antegrade or retrograde direction. The device 10 can be inserted at a radial vessel for access to peripheral vessels in an antegrade direction. The device 10 can be manufactured in smaller and larger sizes depending on the size of the vessel, wherein smaller and larger denote the diameter of the device as measured at the cylindrical tube 26. In some cases, the device 10 can be smaller to be inserted at a radial vessel or another peripheral vessel. In some cases, the device 10 can be larger to be employed above or below the knee.

In some embodiments, the diameter of the device 10 as measured at the cylindrical tube 26 can be from about 1 mm and about 4 mm. The diameter can be from about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 2 mm to about 2.5 mm, about 2 mm to about 3 mm, about 2 mm to about 3.5 mm, about 2 mm to about 4 mm, about 2.5 mm to about 3 mm, about 2.5 mm to about 3.5 mm, about 2.5 mm to about 4 mm, about 3 mm to about 3.5 mm, or from about 3 mm to about 4 mm. In some cases, the diameter is less than about 1 mm, less than about 1.5 mm, less than about 2 mm, less than about 2.5 mm, less than about 3 mm, less than about 3.5 mm, or less than about 4 mm. In some cases, the diameter is greater than about 1 mm, greater than about 1.5 mm, greater than about 2 mm, greater than about 2.5 mm, greater than about 3 mm, greater than about 3.5 mm, or greater than about 4 mm. In some cases, the diameter can be from about 1.6 mm and 2.3 mm, or from about 5 Fr to about 7 Fr. In some cases, the diameter is about 2 mm, about 2.5 mm, or about 3.5 mm.

In some embodiments, the diameter of the tip 18 can be from about 1 mm and about 1.8 mm. In some embodiments, the diameter of the tip 18 can be from about 1 mm to about 1.2 mm, about 1 mm to about 1.4 mm, about 1 mm to about 1.6 mm, about 1 mm to about 1.8 mm, about 1.2 mm to about 1.4 mm, about 1.2 mm to about 1.6 mm, about 1.2 mm to about 1.8 mm, about 1.4 mm to about 1.6 mm, about 1.4 mm to about 1.8 mm, or from about 1.6 mm to about 1.8 mm. In some cases, the diameter can be less than 1 mm, less than 1.2 mm, less than 1.4 mm, less than 1.6 mm, or less than 1.8 mm. In some cases, the diameter can be greater than 1 mm, greater than 1.2 mm, greater than 1.4 mm, greater than 1.6 mm, or greater than 1.8 mm. In some cases, the diameter can be from about 1.1 mm to about 1.35 mm. In some cases, the diameter can be from about 1.1 mm to about 1.15 mm, about 1.1 mm to about 1.2 mm, about 1.1 mm to about 1.25 mm, about 1.1 mm to about 1.3 mm, about 1.1 mm to about 1.35 mm, about 1.15 mm to about 1.2 mm, about 1.15 mm to about 1.25 mm, about 1.15 mm to about 1.3 mm, about 1.15 mm to about 1.35 mm, about 1.2 mm to about 1.25 mm, about 1.2 mm to about 1.3 mm, about 1.2 mm to about 1.35 mm, about 1.25 mm to about 1.3 mm, about 1.25 mm to about 1.35 mm, or from about 1.3 mm to about 1.35 mm.

In some cases, the diameter can be about 1.6 mm. In some cases, the device 10 for antegrade use above the knee can have a diameter of about 3.5 mm and a tip diameter of about 1.6 mm.

In some cases, the device 10 for antegrade use below the knee can have a diameter of about 2.5 mm and a tip diameter of about 1.35 mm. In some cases, the device 10 for antegrade use when inserted into a radial vessel can have a diameter of about 2 mm to about 2.5 mm and a tip diameter of about 1.1 mm to about 1.35 mm.

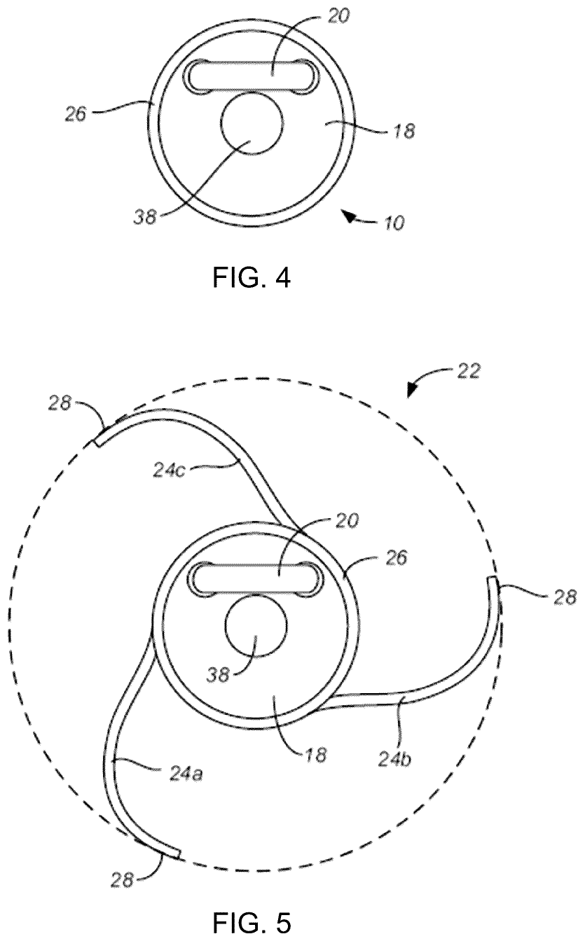

FIG. 4 shows a front view of a distal portion of device 10 shown with a centering cage parallel to and part of cylindrical tube 26 in a radially constrained configuration according to embodiments described herein. FIG. 5 shows a front view of distal portion of device 10 shown with a centering cage 22 in a radially expanded configuration according to embodiments described herein. When radially constrained, the spiral centering springs 24 will generally be collapsed within an envelope defined by the cylindrical tube 26, as shown in FIG. 4. When released from constraint, in contrast, each of the three spiral centering springs 24a, 24b and 24c will radially open to define flat surfaces 28 having an effective diameter shown in broken line. The particular contours and flat surfaces defined by each of the spiral centering springs can be determined by thermal setting of the nitinol or other shape memory alloy during fabrication of the centering cage 22. As also observed in FIG. 5, the leading edge of each of the spiral centering springs 24 has a very low profile which allows the centering springs to be advanced through occlusive material with a reduced resistance to pushing. Also shown are cutting loop 20, tip 18, and distal opening 38. Any variations of tips, centering cages, and devices described below can utilize a similar rotational and expanded functionality.

FIG. 6A depicts a perspective side view of the distal portion of a device 600 in accordance with an embodiment described herein. The distal portion of device 600 can comprise cutting loops 601, tip 602, outer member ring (OM ring) 603, shaft 604, and C ring 40. In some cases, cutting loops 601, tip 602, outer member ring 603, and C ring 40 comprise a metallic zone 610 of the device 600. Metallic zone 610 may be a stiff area of the device 600. Metallic zone 610 may comprise nitinol or other titanium alloys or metal alloys. Outer member ring 603 may be used to hold the shaft 604. Device 600 can be used with handle 16 and other elements of proximal end 14. In some cases, device 600 is a catheter.

A rotatable cutting tip 602 can be located at a distal tip of device 600 and can comprise a cutting loops 601 which may be rotated. In some cases, device 600 can comprise one or more cutting loops 601. In some cases, device 600 can comprise two or more cutting loops 601. In some cases, with reference to FIG. 6A, device 600 comprises two cutting loops 601. As with device 10, the cutting tip 602 and cutting loops 601 can have multiple variations, as described below, depending on the size, location, and density of the occlusion to tunnel through. The cutting tip 602, and thus the cutting loops 601, can be rotated at varying speeds. Although device 600 does not comprise a centering cage, it can still remain centered by the shaft 604, dual cutting loops, and/or the rotating motion of the tip 602. Unlike device 10, device 600 may comprise a single shaft instead of an inner and outer shaft.

In some cases, C ring, or retainer ring, 40 may be welded to outer shaft through an outer member OM ring) ring 603. In some cases, OM ring 603 may be bonded to the outer shaft 604. In some cases, C ring 40 may be or may not be coupled to tip 602. Outer member (OM) and retainer ring 40 can help the shaft 604 to stay near the tip 602. In some cases, the shaft 604 is not otherwise welded or bonded to the tip beyond outer member ring 603. In some cases, there may be an additional coupling mechanism between shaft 604 and tip 602. The combination of outer member ring 603 and C ring 40 may allow the tip 602 to freely rotate with the inner shaft while an outer shaft stays near tip 602. In some cases, as shown in FIG. 6C, there may be more than one shaft.

The device 600 can be employed in coronary arteries in an antegrade direction. The device 600 can be inserted at a pedal vessel for access to peripheral vessels in a retrograde direction. The device 600 can be manufactured in smaller and larger sizes depending on the size of the vessel, wherein smaller and larger denote the diameter of the device as measured at the tip 602. In some cases, the device 600 can be smaller to be employed at a coronary artery. In some cases, the device 600 can be larger to be inserted at a pedal vessel or another peripheral vessel.

In some cases, device 600 may be a separate device. In some cases, device 600 may comprise a base for additional layers, edits, or adjustments as discussed throughout.

In some embodiments, the diameter of device 600 as measured at the tip 602 can be from about 1 mm and about 1.5 mm. The diameter can be from about 1 mm to about 1.1 mm, about 1 mm to about 1.2 mm, about 1 mm to about 1.3 mm, about 1 mm to about 1.4 mm, about 1 mm to about 1.5 mm, about 1.1 mm to about 1.2 mm, about 1.1 mm to about 1.3 mm, about 1.1 mm to about 1.4 mm, about 1.1 mm to about 1.5 mm, about 1.2 mm to about 1.3 mm, about 1.2 mm to about 1.4 mm, about 1.2 mm to about 1.5 mm, about 1.3 mm to about 1.4 mm, about 1.3 mm to about 1.5 mm, or from about 1.4 mm to about 1.5 mm. In some cases, the diameter is less than about 1 mm, less than about 1.1 mm, less than about 1.2 mm, less than about 1.3 mm, less than about 1.4 mm, or less than about 1.5 mm. In some cases, the diameter is greater than about 1 mm, greater than about 1.1 mm, greater than about 1.2 mm, greater than about 1.3 mm, greater than about 1.4 mm, or greater than about 1.5 mm.

In some cases, the diameter is 1.1. The diameter can be 1.35. In some cases, the diameter can be from about 1.1 mm to about 1.35 mm. In some cases, the diameter can be from about 1.1 mm to about 1.15 mm, about 1.1 mm to about 1.2 mm, about 1.1 mm to about 1.25 mm, about 1.1 mm to about 1.3 mm, about 1.1 mm to about 1.35 mm, about 1.15 mm to about 1.2 mm, about 1.15 mm to about 1.25 mm, about 1.15 mm to about 1.3 mm, about 1.15 mm to about 1.35 mm, about 1.2 mm to about 1.25 mm, about 1.2 mm to about 1.3 mm, about 1.2 mm to about 1.35 mm, about 1.25 mm to about 1.3 mm, about 1.25 mm to about 1.35 mm, or from about 1.3 mm to about 1.35 mm.

In some cases, the device 600 for coronary use can have a tip diameter from about 1.1 mm to about 1.35 mm. In some embodiments, the device 600 with pedal access can have a tip diameter of about 1.35.

The device 600 can comprise a working length calculated from a handle to the cutting loop 601 and including an extension of the distal end from the proximal end (e.g. the end comprising the handle). In some cases, the working length can be from about 100 cm and about 180 cm. In some cases, the working length can be from about 100 cm to about 120 cm, about 100 cm to about 140 cm, about 100 cm to about 160 cm, about 100 cm to about 180 cm, about 120 cm to about 140 cm, about 120 cm to about 160 cm, about 120 cm to about 180 cm, about 140 cm to about 160 cm, about 140 cm to about 180 cm, or from about 160 cm to about 180 cm. In some cases, the working length is less than about 100 cm, less than about 120 cm, less than about 140 cm, less than about 160 cm, or less than about 180 cm. In some cases, the working length is greater than about 100 cm, greater than about 120 cm, greater than about 140 cm, greater than about 160 cm, or greater than about 180 cm.

In some cases, the device 600 for coronary use can have a working length from about 145 cm to about 160 cm. In some embodiments, the device 600 with pedal access can have a working length of from about 125 c, to about 145 cm.

The shaft 604 can be rotatable. In some embodiments, the shaft 604 has a lumen which is configured to accommodate a guidewire GW as well as for other purposes, such as infusion, suction, and the advancement of other interventional tools.

In some embodiments, the cutting tip 602 has a central passage which is aligned and contiguous with the lumen of the inner shaft. In some embodiments, the central passage has a distal opening through which a guidewire or other element, tool, or component may be advanced. In some cases, the cutting tip 602 is fixedly attached to a distal end of the shaft 604 so that rotation of the shaft 604 will cause rotation of the cutting tip 602 as well as the cutting loops 601.

Device 600 can be flexible. In some cases, this is due to a single, thinner shaft instead of multiple layers of shafts. In some cases, this is due to a lack of a centering cage. In some cases, device 600 can be advantageous when flexibility is helpful, such as in tortuous anatomy of the pedal vessels and coronary arteries. Device 600 can be deployed in both straight and angled directions.

FIG. 6B shows a similar device 620 to device 600. Device 620 comprises cutting loops 621, tip 622, retaining ring 623, outer shaft 624, and metallic zone 630. FIG. 6C shows a length-wise cross-section of device 620 comprising cutting loops 621, tip 622, retaining ring 623, outer shaft 624, inner shaft 625, shaft gap 626, C ring hole 627, and metallic zone 630. As discussed above, retaining ring 623 can be attached to outer shaft 624. As discussed above, tip 622 can be attached to inner shaft 625. C ring hole 627 can match the size and/or other dimensions of C ring, especially the smooth exterior of C ring. The C ring and C ring hole 627 can act as intermediaries between the outer shaft and OM ring and the inner shaft and tip, such that the outer shaft remains near the tip without hindering the rotation of the inner shaft and tip. Furthermore, as shown in FIG. 6C by the existence of C ring hole 627, the retaining ring is not attached to the tip but may just rest along the circumference of the proximal end of the tip 622.

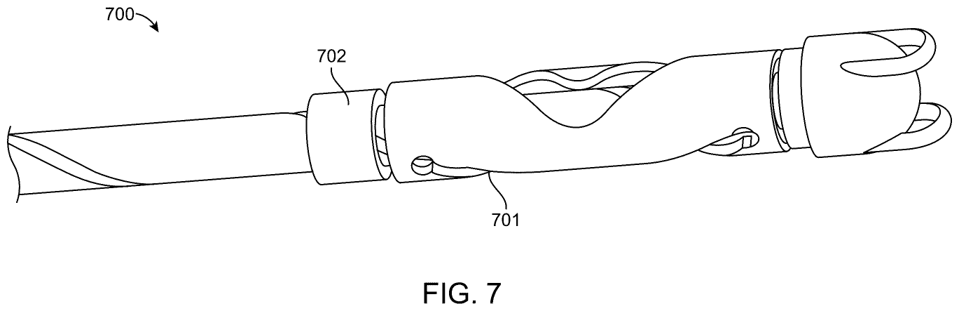

In some cases, with reference to FIG. 7, device 700 can comprise only the inner shaft of the device 600 (no outer shaft) with the addition of a centering cage 701. Device 700 may comprise a retainer band 702, which is attached to the outer surface near the distal end of the inner shaft. This may be different from the retaining rings described herein. In some cases, the retainer band 702 may limit or prevent proximal movement of the centering cage. The centering cage can have the elements, functionality, and dimensions discussed above regarding centering cage 22. In some cases, the centering cage may have a smaller length than the dimensions discussed above regarding centering cage 22. In some embodiments, there is no outer shaft, hence the centering cage is freely rotatable over the inner shaft. The proximal-distal movement of this floatable centering cage may be limited by the tip distally and retainer band 702 proximally.

The floatable aspect of the centering cage 701 may provide freedom of movement to the centering cage 701. The floatable aspect of the centering cage 701 may provide about 1 mm of freedom of movement to the centering cage 701. The floatable aspect of the centering cage 701 may provide less than about 0.2 mm, less than about 0.4 mm, less than about 0.6 mm, less than about 0.8 mm, or less than about 1 mm of freedom of movement to the centering cage 701. The floatable aspect of the centering cage 701 may provide greater than about 0 mm, greater than about 0.2 mm, greater than about 0.4 mm, greater than about 0.6 mm, greater than about 0.8 mm, or greater than about 1 mm of freedom of movement to the centering cage 701. The floatable aspect of the centering cage 701 may provide from about 0 mm to about 1 mm of freedom of movement to the centering cage 701. The floatable aspect of the centering cage 701 may provide from about 0 mm to about 0.2 mm, about 0 mm to about 0.4 mm, about 0 mm to about 0.6 mm, about 0 mm to about 0.8 mm, about 0 mm to about 1 mm, about 0.2 mm to about 0.4 mm, about 0.2 mm to about 0.6 mm, about 0.2 mm to about 0.8 mm, about 0.2 mm to about 1 mm, about 0.4 mm to about 0.6 mm, about 0.4 mm to about 0.8 mm, about 0.4 mm to about 1 mm, about 0.6 mm to about 0.8 mm, about 0.6 mm to about 1 mm, or from about 0.8 mm to about 1 mm of freedom of movement to the centering cage 701.

Adding a centering cage can be helpful in increasing the stability of the device. In some cases, increasing stability can increase the device's ability to stay centered in a vessel, which can be helpful if the device is large relative to the vessel size. This can help avoid damaging the vessel wall. All other features of device 700 can be the same as device 600.

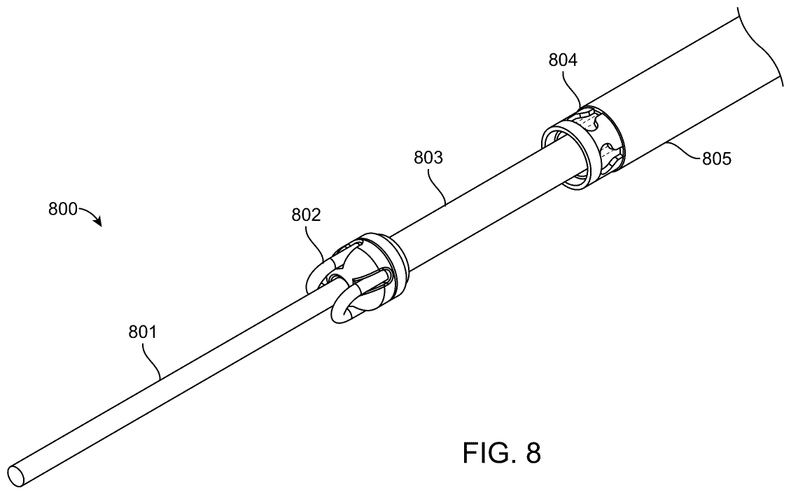

FIG. 8A shows a perspective view of a telescoping feature of the distal portion of a device in accordance with an embodiment described herein. In some cases, with reference to FIG. 8A, device 800 can comprise device 600 with the addition of a telescoping feature. In some cases, device 800 can comprise an alternate device, for example device 10 or 600, with the addition of a telescoping feature. Device 800 can comprise outer shaft 805, ring 804, inner shaft 803, tip 802, and guidewire 801. All other features of device 800 can be the same as device 600.

The tip 802 can be telescoped by extending the inner shaft 803. In some cases, the tip can be telescoped dynamically, such that there are multiple lengths of telescoping that a user can control. In some cases, the tip is telescoped fixedly, such that it extends from the outer shaft 805 by a preset amount. In some cases, telescopic device 800 can be beneficial in navigating complex tortuous anatomy and/or branch points. This is because the inflection point of the distal end of device 800 shifts closer to the tip, thereby improving flexibility of the distal end. Put another way, the part of the device navigating difficult vasculature may be the inner shaft 803 rather than, or in addition to, the outer shaft 805. This can provide “at will” flexibility based on the vasculature the user is traversing. This can further be assisted by the size of the inner shaft 803. In some cases, inner shaft 803 has a smaller diameter than other shafts, for example shaft 604. This smaller diameter can also improve navigation. The improved flexibility and navigation abilities can be especially helpful in the complex vessels of the pedal access points and the coronary arteries. In devices 800 used with handles 14 with the wheel 44 disposed on a shuttle, a user may have the benefit of both flexibility and the added strength or force of the device to tunnel through harder occlusions.

In some cases, device 800 may vary from a device such as device 600 by lacking a C ring. In some cases, there may be an alternate structure or ring at the proximal end of the tip 802. In some cases, tip 802 may not have an alternate structure or ring and may have a smaller metallic section.

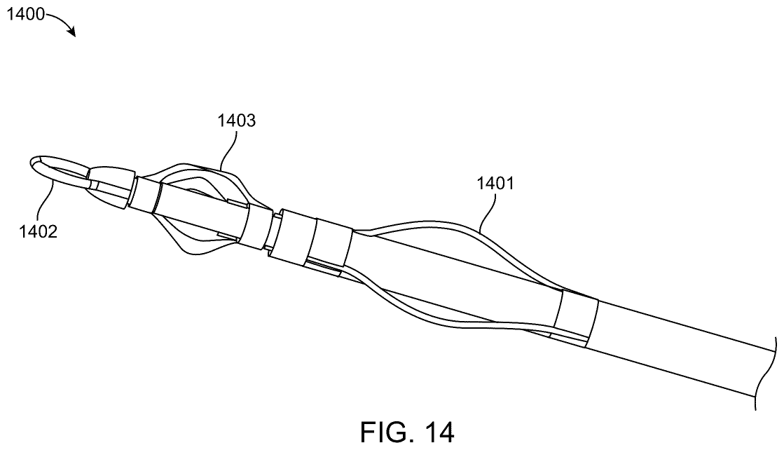

In some cases, device 800 may have a centering element or centering cage. In some cases, similar to device 10, the centering element may be on outer shaft 805. In some cases, the centering element may be on telescoping inner shaft 803. In some cases, as in FIG. 14 described below, there may be a centering element on both outer shaft 805 and inner shaft 803. In some cases, regardless of whether there is an additional centering element on the outer shaft, the centering element on inner shaft 803 may be smaller than centering systems on the outer shaft. This size relationship may likewise be similar to FIG. 14. The size of the inner shaft centering element may be similar to the centering cage on device 700.

The distance that the guidewire 801 protrudes from the device may affect the use of the device in certain situations or types of vasculature. In some cases, it may be advantageous to extend the guidewire 801 farther to navigate through more difficult bends and branching points. In some cases, it may be advantageous to keep the guidewire 801 near tip 802. In some cases, when the guidewire is extended farther, the guidewire 801 may be retracted fully into the device once the device is past the difficult areas. In some cases, the guidewire 801 may remain protruding by a few millimeters past the cutting loop(s). In some cases, the guidewire 801 protrudes by less than about 0.5 mm, less than about 1.0 mm, less than about 1.5 mm, less than about 2.0 mm, or less than about 2.5 mm. In some cases, the guidewire protrudes by greater than about 0.5 mm, greater than about 1.0 mm, greater than about 1.5 mm, greater than about 2.0 mm, or greater than about 2.5 mm. In some cases, the guidewire 801 can protrude by from about 0.5 mm to about 1.0 mm, about 0.5 mm to about 1.5 mm, about 0.5 mm to about 2.0 mm, about 0.5 mm to about 2.5 mm, about 1.0 mm to about 1.5 mm, about 1.0 mm to about 2.0 mm, about 1.0 mm to about 2.5 mm, about 1.5 mm to about 2.0 mm, about 1.5 mm to about 2.5 mm, or from about 2.0 mm to about 2.5 mm. When the guidewire 801 protrudes by a short distance, it may be able to use the force of the catheter device 800. In this way, the effect or strength of guidewire 801 can be increased without having a stiffer wire, thereby retaining flexibility when the wire extends far and retaining strength when the wire protrudes a few millimeters from the cutting loop. By supporting the guidewire 801, the tip 802 and the inner shaft 803 may act similar to a single strong wire with 804 as the beginning of the catheter device following the wire.

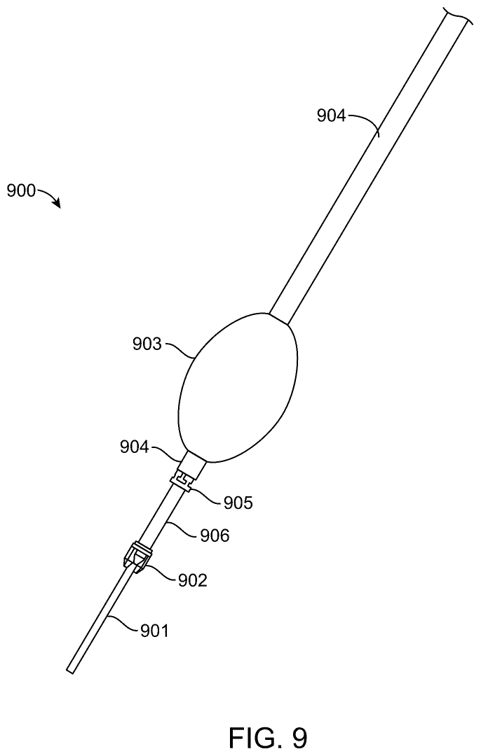

FIG. 9 shows a perspective view of a telescoping feature of the distal portion of a device with an additional feature, such as a balloon in accordance with an embodiment described herein. In some cases, with reference to FIG. 9, device 900 can comprise device 10, 600, or 700 with the addition of a telescoping feature. Device 900 can comprise outer shaft 904, balloon 903, tip 902, guidewire 901, ring 905, and inner shaft 906. In some embodiments, the outer shaft of an example telescoping device can comprise an anchoring balloon 903 near the distal end. In some cases, the anchoring balloon 903 can be another anchoring element. The anchoring element can be a mechanical anchor that grips the inner wall of the blood vessel. The anchoring element can be a friction-based anchor. The anchor can be expandable. The anchor can be an expandable balloon. The anchoring balloon can inflate when expanded. When expanded, the anchoring balloon 903 can firmly anchor the device 900 by anchoring outer shaft 904 to the inner surface of the target vessel. The anchoring mechanism 903 can stabilize the device. The anchoring balloon 903 can prevent backup when the inner shaft and tip is advanced through the occlusive plaque and/or occlusion mass, in particular hard occlusions. The anchoring balloon may also be used to fracture hard plaque and/or occlusion mass at the proximal cap or body of the CTO and facilitate the advancement of shaft.

In some cases, anchoring balloon 903 can be located from about 2 cm to about 5 cm away from the tip 902. In some cases, anchoring balloon 903 can be located from about 2 cm to about 2.5 cm, about 2 cm to about 3 cm, about 2 cm to about 3.5 cm, about 2 cm to about 4 cm, about 2 cm to about 4.5 cm, about 2 cm to about 5 cm, about 2.5 cm to about 3 cm, about 2.5 cm to about 3.5 cm, about 2.5 cm to about 4 cm, about 2.5 cm to about 4.5 cm, about 2.5 cm to about 5 cm, about 3 cm to about 3.5 cm, about 3 cm to about 4 cm, about 3 cm to about 4.5 cm, about 3 cm to about 5 cm, about 3.5 cm to about 4 cm, about 3.5 cm to about 4.5 cm, about 3.5 cm to about 5 cm, about 4 cm to about 4.5 cm, about 4 cm to about 5 cm, or from about 4.5 cm to about 5 cm from the tip 902. In some cases, anchoring balloon 903 can be located less than about 2 cm, less than about 2.5 cm, less than about 3 cm, less than about 3.5 cm, less than about 4 cm, less than about 4.5 cm, or less than about 5 cm. In some cases, anchoring balloon 903 can be located greater than about 2 cm, greater than about 2.5 cm, greater than about 3 cm, greater than about 3.5 cm, greater than about 4 cm, greater than about 4.5 cm, or greater than about 5 cm.

When inflated, anchoring balloon 903 can be from about 1 mm to about 3 mm in diameter. Anchoring balloon 903 can be from about 1 mm to about 1.4 mm, about 1 mm to about 1.9 mm, about 1 mm to about 2.2 mm, about 1 mm to about 2.6 mm, about 1 mm to about 3 mm, about 1.4 mm to about 1.9 mm, about 1.4 mm to about 2.2 mm, about 1.4 mm to about 2.6 mm, about 1.4 mm to about 3 mm, about 1.9 mm to about 2.2 mm, about 1.9 mm to about 2.6 mm, about 1.9 mm to about 3 mm, about 2.2 mm to about 2.6 mm, about 2.2 mm to about 3 mm, or from about 2.6 mm to about 3 mm in diameter when inflated. Anchoring balloon 903 can be less than about 1 mm, less than about 1.4 mm, less than about 1.9 mm, less than about 2.2 mm, less than about 2.6 mm, less than about 3 mm in diameter when inflated. Anchoring balloon 903 can be greater than about 1 mm, greater than about 1.4 mm, greater than about 1.9 mm, greater than about 2.2 mm, greater than about 2.6 mm, greater than about 3 mm in diameter when inflated.

### Tips

Disclosed herein are a variety of tips and cutting loops for use in the devices disclosed herein. In some cases, a certain tip is preferable depending on the nature and contents of the occlusion, for example the length, location, and/or hardness of the occlusion. For example, an occlusion in a smaller vessel may benefit from a narrowing tip and cutting loop.

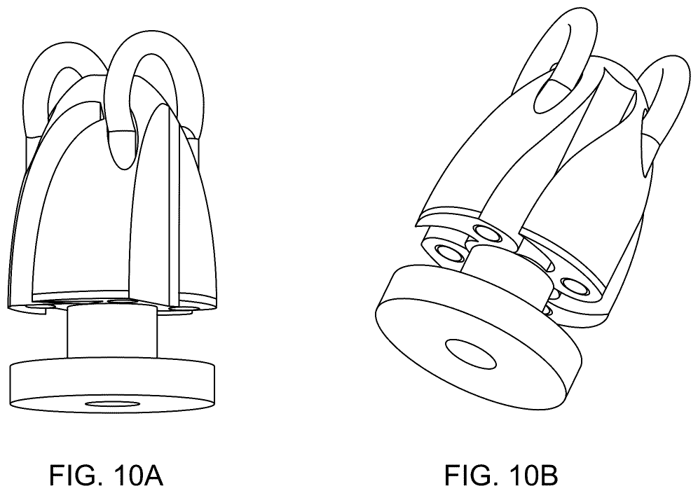

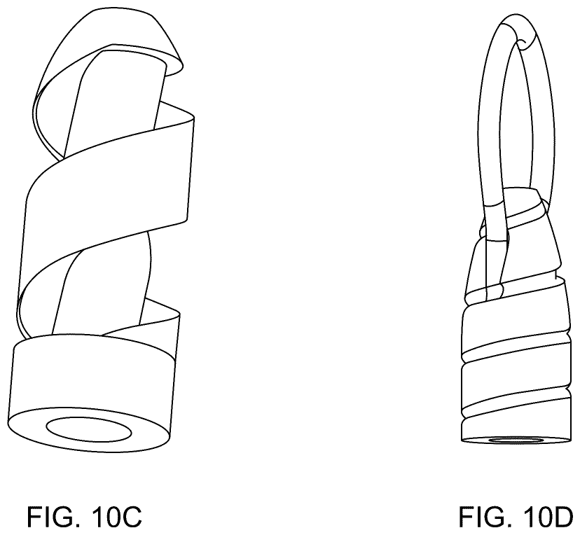

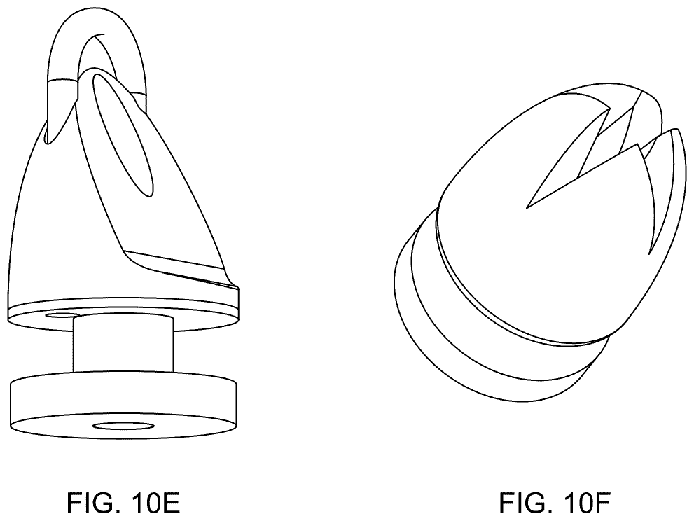

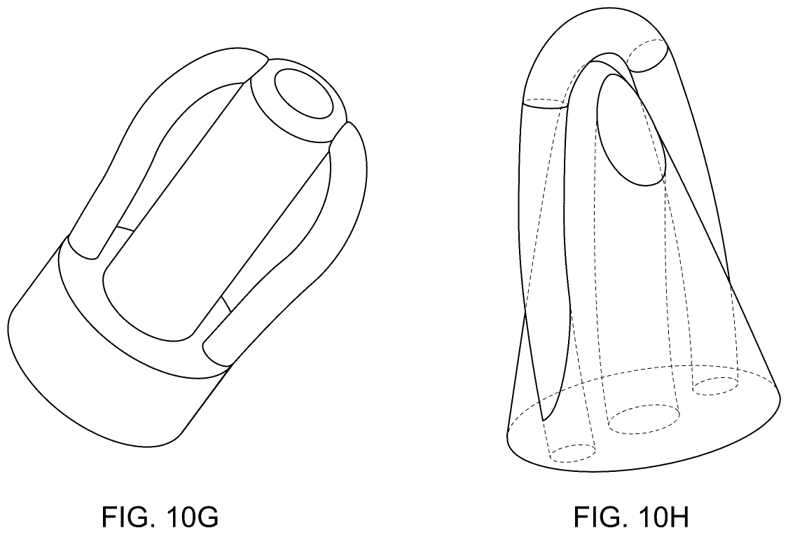

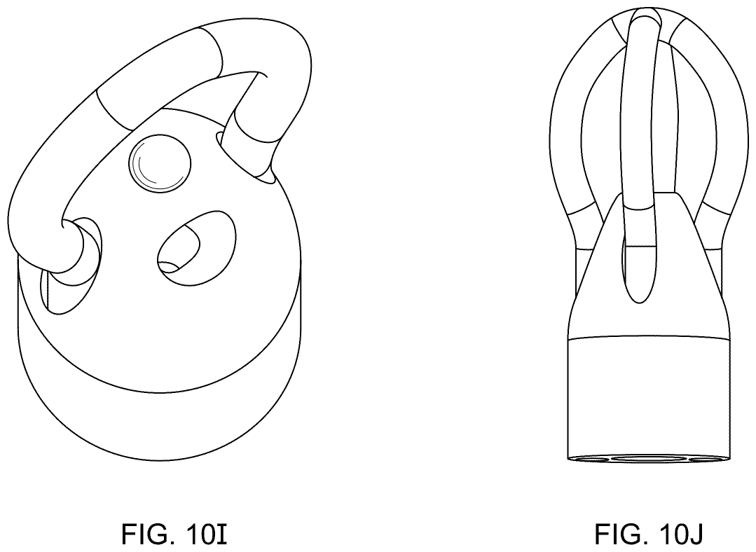

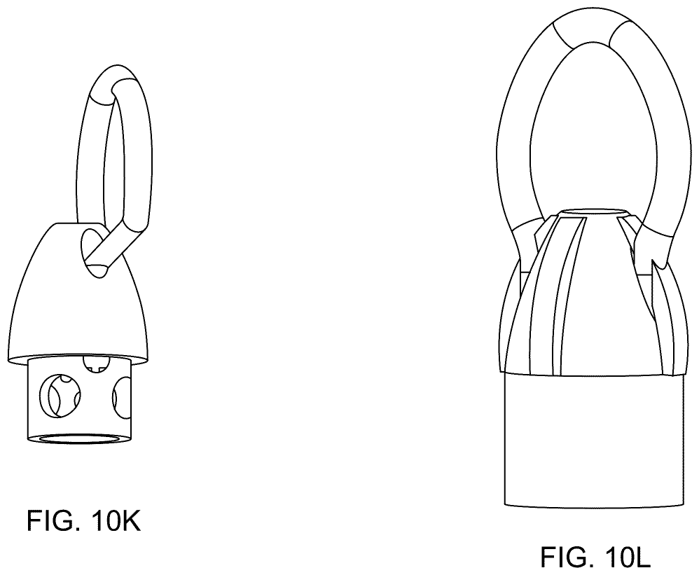

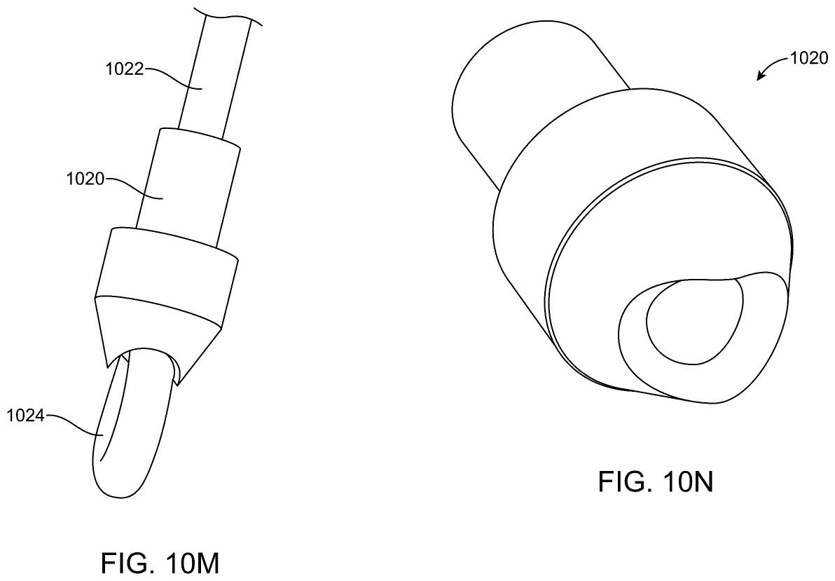

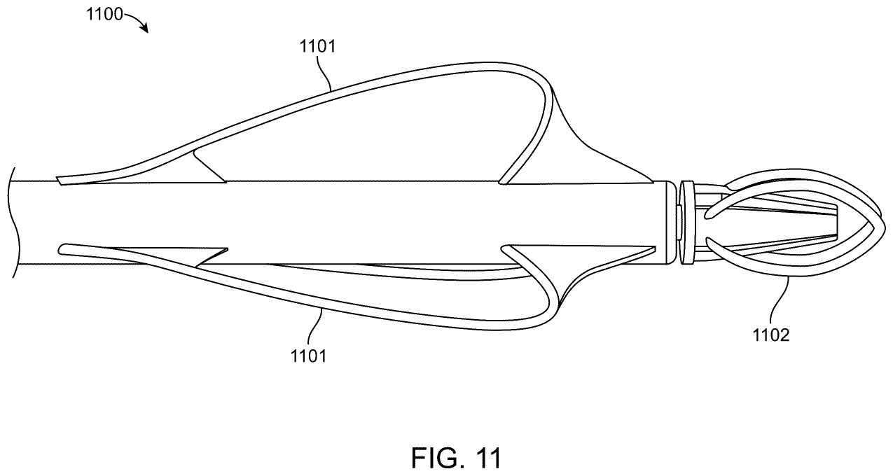

FIGS. 10A-10N show side and perspective views of example tip variations that can be used with any of the devices described herein.

FIG. 10A shows a side view of an example tip variation that can be used with any of the devices described herein. The tip and cutting loops shown in FIG. 10A comprise a flywheel fluted tip that may be designed for crossing hard and/or calcified occlusions, as well as softer occlusions. The tip can comprise flutes, grooves, channels or ridges with sharp edges. Those flutes may engage and pierce or cut the calcified plaque and/or occlusion mass. The cutting loops may be located at the advancing end of the flutes to protect from inadvertent vessel wall damage by the sharp ends of the flutes. In some cases, this design can comprise one cutting loop. In some cases, this design can comprise more than one cutting loop, for example two cutting loops. This tip can have a neck between the base region attached to the rest of the device and the actual tip region with the flutes and attached to the cutting loop.

FIG. 10B shows a bottom-up perspective view of an example tip variation that can be used with any of the devices described herein. The tip and cutting loop shown in FIG. 10B comprise a flywheel fluted tip that may be designed for crossing hard and/or calcified occlusions, as well as softer occlusions. The tip can comprise flutes, grooves, channels or ridges with sharp edges. Those flutes may engage and pierce or cut the calcified plaque and/or occlusion mass. The cutting loops may be located at the advancing end of the flutes to protect from inadvertent vessel wall damage by the sharp ends of the flutes. Although similar to the tip of FIG. 10A, FIG. 10B may have helically oriented flutes along the long axis of the tip. This orientation can work in tandem with the torque and rotational movement of the tip during its advancement into an occlusion. In some cases, this design can comprise one cutting loop. In some cases, this design can comprise more than one cutting loop, for example two cutting loops. This tip can have a neck between the base region attached to the rest of the device and the actual tip region with the flutes and attached to the cutting loop. The guidewire lumen can be visible near the bottom of the figure.

FIG. 10C shows a side view of an example tip variation that can be used with any of the devices described herein. The tip and cutting loop shown in FIG. 10C comprise a helical screw tip that may be designed for crossing hard and/or calcified occlusions, as well as softer occlusions. The helical cut on the tip surface can work in tandem with the torque and rotational movement of the tip during its advancement into an occlusion. This design can be safe for the vessel walls because the sharp edges cut only the plaque and/or occlusion mass at the advancing end. In some cases, this tip does not comprise cutting loops. In some cases, this tip can comprise cutting loops. In some cases, the tip can comprise one, two, or more cutting loops.

FIG. 10D shows a side view of an example tip variation that can be used with any of the devices described herein. The tip and cutting loop shown in FIG. 10D comprise a helical screw tip that may be designed for crossing hard and/or calcified occlusions, as well as softer occlusions. The helical cut on the tip surface can work in tandem with the torque and rotational movement of the tip during its advancement into an occlusion. This design can be safe for the vessel walls because the sharp edges cut only the plaque and/or occlusion mass at the advancing end. In some cases, this tip does not comprise cutting loops. In some cases, this tip can comprise cutting loops. In some cases, the tip can comprise one, two, or more cutting loops. The screws in this design can be shallower than those of the design of FIG. 10C.

FIG. 10E shows a side view of an example tip variation that can be used with any of the devices described herein. The tip and cutting loop shown in FIG. 10E comprise a pointed-end tip that may be designed for crossing hard and/or calcified occlusions, as well as softer occlusions. The sharp pointed tip may facilitate effective piercing and advancement of the device through a plaque and/or occlusion mass. The sloping edge may facilitate effective piercing and advancement of the device through a plaque and/or occlusion mass. The cutting loop can be positioned proximal and near to the sharp tip end to protect the vessel from inadvertent injury and perforation due to the device. In some cases, this tip does not comprise cutting loops. In some cases, this tip can comprise cutting loops. In some cases, the tip can comprise one, two, or more cutting loops.

FIG. 10F shows a top-down perspective view of an example tip variation that can be used with any of the devices described herein. The tip and cutting loop shown in FIG. 10F comprise a serrated tip that may be designed for crossing hard and/or calcified occlusions, as well as softer occlusions. The serrations can facilitate effective piercing and advancement of the device through harder plaque and/or occlusion mass. The distal ends of the serrations can be pointed inward to protect the vessel from inadvertent injury and perforation due to the device. In some cases, this tip design does not comprise cutting loops. In some cases, this tip design can comprise cutting loops.

FIG. 10G shows a perspective view of an example tip variation that can be used with any of the devices described herein. The tip and cutting loop shown in FIG. 10G comprise a tubular tip that may be designed for crossing moderately hard and/or moderately calcified occlusions, as well as softer occlusions. This tip can have a smaller cross-sectional area, which may result in lesser drag. The smaller cross-sectional area can also increase the mean force exerted over a given area on the plaque and/or occlusion mass, leading to an increase in cutting power. The smaller cross-sectional area can make this tip applicable for smaller vessels. The unique cutting loops are oriented in a way that can improve the tip's cutting power. In some cases, this design may protect from guidewire jailing. The cutting loops can also prevent inadvertent subintimal passage and trauma to the vessel walls. In some cases, the tip can comprise one, two, or more cutting loops.

FIG. 10H shows a side view of an example tip variation that can be used with any of the devices described herein. The tip and cutting loop shown in FIG. 10H comprise a cone shaped tip that may be designed for crossing hard and/or calcified occlusions, as well as softer occlusions. This tip can have a smaller cross-sectional area, wherein effectiveness is determined by the area of the tip that is directly involved in cutting into the plaque and/or occlusion mass. The tip can increase the mean force exerted over a given area on the plaque and/or occlusion mass due to the pointed advancing face, leading to an increase in cutting power. The smaller cross-sectional area can also result in decreased drag forces over the tip. In some cases, the guidewire exit hole and one or more cutting loops can be oriented to boost the tip's cutting power and protect against guidewire jailing.

FIG. 10I shows a perspective top-down view of an example tip variation that can be used with any of the devices described herein. The tip and cutting loop shown in FIG. 10I comprise a tip with off-center guidewire exit holes that may be designed for crossing moderately hard and/or moderately calcified occlusions, as well as softer occlusions. If the tip inadvertently approaches and/or gets close to the vessel wall while crossing the occlusion, a guidewire can be advanced out of the off-center tip toward the central axis, which may act as a steering feature. This tip can help to prevent inadvertent subintimal entry. In some cases, this tip can comprise cutting loops. In some cases, the tip can comprise one, two, or more cutting loops.

FIG. 10J shows a side view of an example tip variation that can be used with any of the devices described herein. The tip and cutting loop shown in FIG. 10J comprise a tip with crossed cutting loops that may be designed for crossing moderately hard and/or moderately calcified occlusions, as well as softer occlusions. The cutting loops may comprise four legs, or two full loops forming an “x” when viewed from the top. In some cases, the cutting loops comprise three legs. The guidewire exit hold can be off-centered between and/or below the cutting loop legs. The cross or crown tip can have both a cutting and a centering functionality. As such, it can work well with devices 600 or 800, which do not have centering cages. This tip can have a reduced crossing diameter and/or profile and thus reduce the drag over the device tip. Furthermore, the reduced diameter can improve device and tip movement and/or tracking over narrower curves and angles in tortuous anatomy as well as branch points. As device 600 is frequently used in tortuous anatomy, this tip can complement the design of the rest of the device.

FIG. 10K shows a side view of an example tip variation that can be used with any of the devices described herein. The tip and cutting loop shown in FIG. 10K comprise a tip with a curved cutting loop that may be designed for crossing hard and/or calcified occlusions, as well as softer occlusions. The curved cutting loop can increase the size and/or profile of the tip while still preventing subintimal passage. In some cases, this tip can be used for heavily calcified occlusions. In some embodiments, this tip can be used when there is an eccentric highly calcified area, for example a boulder of calcium, with one-sided, eccentric soft plaque and/or occlusion mass. The curved loop can steer the device tip through the eccentric channel of soft plaque and/or occlusion mass. In some cases, this tip can have one cutting loop. In some cases, it has two cutting loops.

FIG. 10L shows a side view of an example tip variation that can be used with any of the devices described herein. The tip and cutting loop shown in FIG. 10L comprise a tip with a screw surface that may be designed for crossing moderately hard and/or moderately calcified occlusions, as well as softer occlusions. The tip can comprise flutes, grooves, channels or ridges with sharp edges. Those flutes may engage and pierce or cut the calcified plaque and/or occlusion mass. However, unlike the tips of FIGS. 10A-B, the flutes in FIG. 10L can extend outward from the body of the tip, rather than cutting into the body of the tip. The cutting loops may be located at the advancing end of the flutes to protect from inadvertent vessel wall damage by the sharp ends of the flutes. In some cases, the tip can comprise one, two, or more cutting loops.

FIG. 10M shows a perspective view of an example tip variation that can be used with any of the devices described herein. FIG. 10N shows a bottom-up perspective view of the example tip of FIG. 10M without a guidewire. The tip and cutting loops shown in FIG. 10M-10N comprise an “eyelet” model tip that may be designed for crossing hard and/or calcified occlusions, as well as softer occlusions. In some embodiments, the guidewire tip 1022 can be incorporated with a cutting loop 1024. The device tip 1020 can be carved out into an eyelet to snugly fit the cutting loop during a procedure involving the devices and methods disclosed herein. In some cases, both the guidewire and the device are advanced together through the occlusion. This can be beneficial in moderately calcified plaque and/or occlusion mass or highly calcified plaque and/or occlusion mass with softer areas, as the guidewire loop 1024 can be advanced beyond the tip and/or telescoped through the softer plaque and/or occlusion mass areas.

### Centering Cage

Disclosed herein are a variety of centering cages or centering elements for use in the devices disclosed herein. In some cases, the default centering cage for such devices is that shown in FIGS. 2-3A comprising three spirals. In some cases, an alternate centering cage may be used depending on the condition of the occlusion, for example the length, location, and/or hardness of the occlusion.

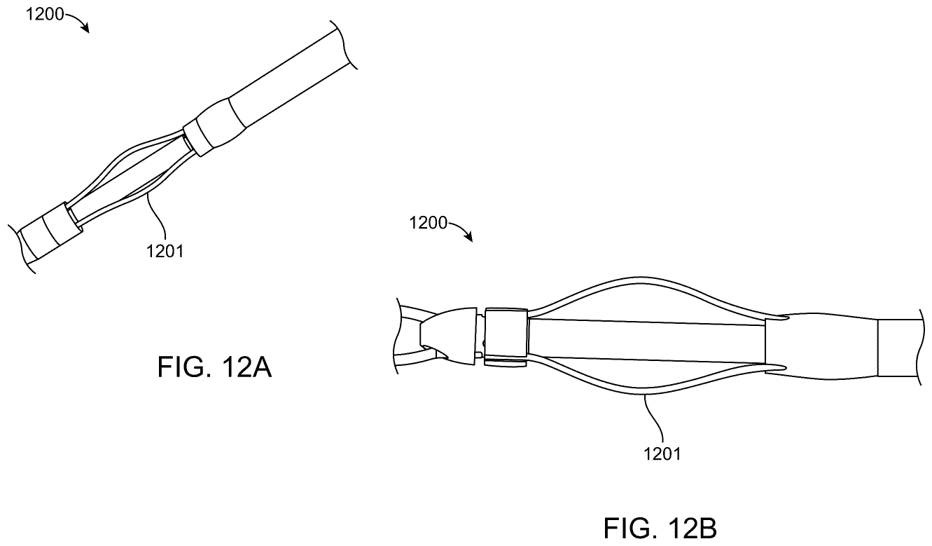

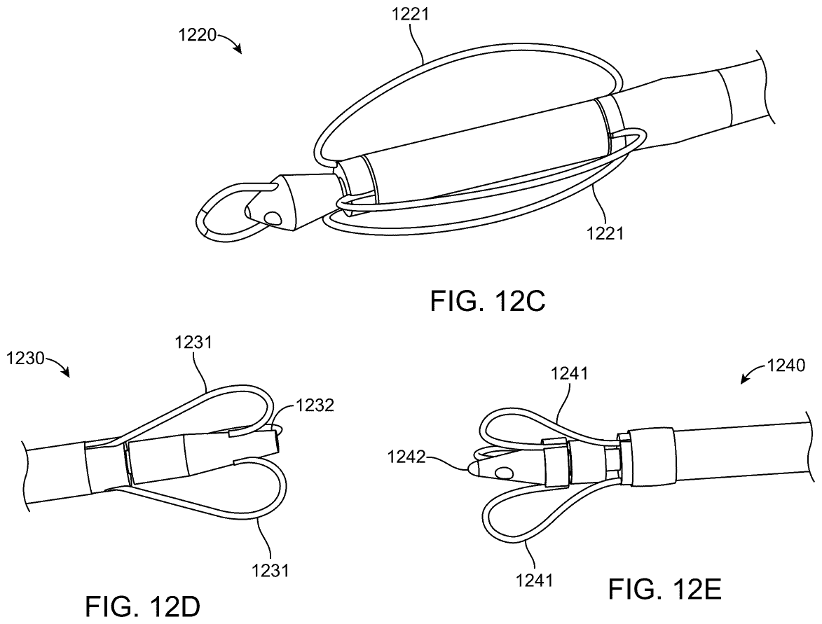

FIG. 12 shows a side view of tangential centering cage on a device as described herein in accordance with an embodiment described herein. Device 1200 can comprise tip with cutting loop 1202 and centering cage 1201. Centering cage 1201 can be two dimensional. Centering cage 1201 can comprise loops tangential to the body of the device 1200, such that they only go in a forward-backward direction instead of forming spirals. In some cases, most of the central portions of the centering cage have been removed relative to the default design. However, centering cage 1201 still retains rounded edges, as seen in the side view of FIG. 12, to avoid damaging the vessel wall. In some cases, the centering cage can comprise 1, 2, 3, 4, 5, or more centering elements. In some cases, device 1200 has a lesser surface area, thereby reducing drag while crossing an occlusion.

Dimensions of tip 1202 and device 1200 can be similar to devices 10 and 600. Device 1200 can comprise any of the devices described herein along with the dimensions of those devices. Tip 1202 can comprise any of the tips described herein.

FIGS. 12A-12C show perspective views (12A and 12C) and side views (12B, 12D, 12E) of retractable centering cages on the distal ends of devices as described herein in accordance with embodiments described herein. Retractable centering systems can be retracted via the handle by the user as desired. Once retracted, the diameter and/or profile of the distal end of any device using these centering cages can decrease significantly, allowing for lesser drag. This can be helpful in crossing harder occlusions. This can also be useful in navigating through smaller size vessels, as the centering system's diameter can be adjusted to fit a vessel of variable diameters. In some cases, this can be helpful in crossing long lesions with significant differences in the size of the vessel from the proximal to the distal end of the lesion.

The retractable centering system can be retracted to change the diameter of the device anywhere from about 2 mm to about 10 mm. This can make a device with a retractable centering system usable in the majority of vessels. In some cases, the retractable centering can be retracted to change the diameter of the device anywhere from about 2 mm to about 4 mm, about 2 mm to about 6 mm, about 2 mm to about 8 mm, about 2 mm to about 10 mm, about 4 mm to about 6 mm, about 4 mm to about 8 mm, about 4 mm to about 10 mm, about 6 mm to about 8 mm, about 6 mm to about 10 mm, or from about 8 mm to about 10 mm. In some cases, the retractable centering can be retracted to change the diameter of the device to less than 2 mm, less than 4 mm, less than 6 mm, less than 8 mm, or less than 10 mm. In some cases, the retractable centering can be retracted to change the diameter of the device to greater than 2 mm, greater than 4 mm, greater than 6 mm, greater than 8 mm, or greater than 10 mm.

FIG. 12A shows a perspective view of device 1200 with a closed retractable centering system 1201. Closed retractable centering system 1201 can comprise outer shaft retaining ring 1202 and distal tip ring 1203. In some cases, outer shaft retaining ring 1202 can attach the proximal end of the closed retractable centering cage of a closed retractable centering system 1201. In some cases, distal tip ring 1203 can attach the distal end of the closed retractable centering cage of a closed retractable centering system 1201. The distal end of the centering cage can be on the tip or proximal to the tip. In some embodiments, the centering system 1201 is attached to the retainer ring. In some cases, a smaller diameter of the centering system 1201 indicates a closed or inactive state. Device 1200 can comprise any of the devices described herein along with the dimensions of those devices. Device 1200 can comprise any of the tips described herein.

FIG. 12B shows a side view of device 1200 with an open retractable centering system 1201. In some embodiments, the centering system 1201 is attached to the retainer ring. In some cases, a larger diameter of the centering system 1201 indicates an open or active state. Centering system 1201 comprises one or more loops. Centering system 1201 can comprise 1, 2, 3, 4, 5, or more loops. In some cases, the loops are evenly spaced apart along the circumference of device 1200.

FIG. 12C shows a perspective view of device 1220 with open retractable centering cage 1221. Device 1220 can comprise any of the devices described herein along with the dimensions of those devices. Device 1220 can comprise any of the tips described herein. In some embodiments, the centering cage is attached to a retainer ring. In some cases, the centering cage comprises forward-looking loops when activated. Centering system 1221 comprises one or more loops. Centering system 1221 can comprise 1, 2, 3, 4, 5, or more loops. In some cases, the loops are evenly spaced apart along the circumference of device 1220.

FIG. 12D shows a side view of device 1230 with open retractable centering cage 1231 and tip 1232. Device 1230 can comprise any of the devices described herein along with the dimensions of those devices. Tip 1232 can comprise any of the tips described herein which has guidewire exit holes on the distal end of the tip. In some cases, the centering system is attached and actuated by the tip 1232 and forward-looking loops. The forward-looking loops can protect the tip which penetrating hard occlusions with a stiff end guidewire. Centering system 1231 comprises one or more loops. Centering system 1231 can comprise 1, 2, 3, 4, 5, or more loops. In some cases, the loops are evenly spaced apart along the circumference of device 1230.

FIG. 12E shows a side view of device 1240 with open retractable centering cage 1241 and tip 1242. Device 1240 can comprise any of the devices described herein along with the dimensions of those devices. Tip 1242 can comprise any of the tips described herein which has guidewire exit holes on the side of the tip. In some cases, the centering system is attached and actuated by the tip 1242 and forward-looking loops. The forward-looking loops can protect the tip which penetrating hard occlusions with a stiff end guidewire. Centering system 1241 comprises one or more loops. Centering system 1241 can comprise 1, 2, 3, 4, 5, or more loops. In some cases, the loops are evenly spaced apart along the circumference of device 1240.

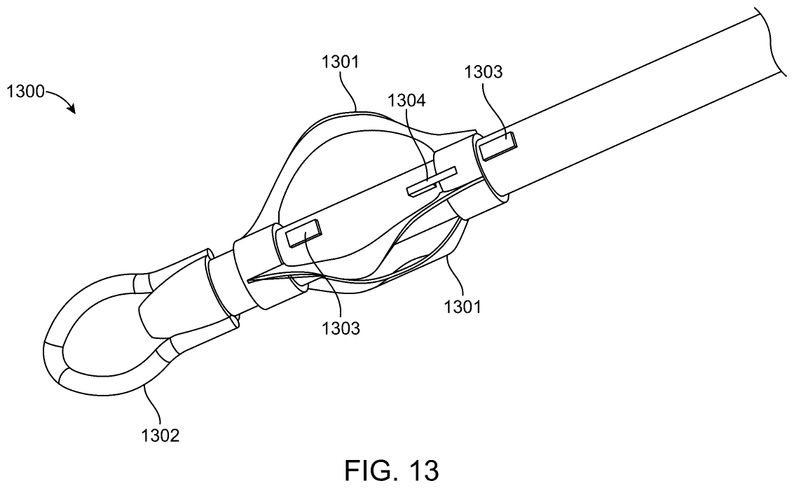

FIG. 13 shows a perspective view of a floating centering cage on the distal end of a device 1300 described herein in accordance with embodiments described herein. Device 1300 comprises floating centering cage 1301, tip 1302, restraining tabs 1303, and clip 1304. Device 1300 can comprise any of the devices described herein along with the dimensions of those devices. Tip 1302 can comprise any of the tips described herein. In some cases, the floating centering cage 1301 can be used in the presence of an inner and outer shaft. In some cases, the floating centering cage 1301 can be used without an outer shaft and only comprises the inner shaft. The inner shaft may be rotatable. In some cases, the floating centering cage 1301 can be used in the place of a restraining ring. In some cases, the centering cage is considered free floating because it is not attached to either the shaft or a retainer ring. This design can also be considered free floating because it is able to be rotated. For example, it can avoid calcified boulders inside the occlusion or in the vessel en route to the occlusion by being rotated. In some cases, the floating centering cage 1301 can have some of the features of the other centering cages, for example a flat spiral design, retractability, tangential loops, etc. In some cases, a device 1300 can have a smaller diameter and thus have lesser drag due to the lack of an outer shift and/or retaining ring.

Centering cage 1301 can comprise bands encircling the device 1300 shaft. These bands can be where the loops of the centering cage come together and attach to the rest of the device. In some cases, there is only one band near the tip 1302. In some cases, there are two or more bands, wherein one band is near the tip 1302 and the other band(s) are proximal to the first band. The band near the tip 1302 is disposed between a restraining tab 1303 and the tip 1302. In some cases, there are as many bands as there are restraining tabs 1303. Centering cage 1301 can comprise one or more restraining tabs 1303. Restraining tabs 1303 can comprise blockers attached to the shaft (for example, glued, soldiered, etc.). The thickness of the restraining tabs 1303 can be greater than the thickness of the bands, such that the restraining tabs 1303 fully block the bands. In some cases, there is only one restraining tab 1303 near the tip 1302. In some cases, there are two or more restraining tabs, wherein one tab is near the tip 1302, e.g. the distal tab, and the other tab(s) are proximal to the first tab, e.g. the proximal tab. The tab near the tip 1302 is configured to keep the distal band between the distal tab and the tip 1302. For the distal band, the role of the tip is played by clip 1304. Clip 1304 can limit the centering cage's ability to move distally, similarly to the tip 1302 for the distal band. In some cases, clip 1304 is unnecessary and just one set of localization features (e.g. the distal tab 1303 and the tip 1302) is sufficient to keep the centering cage and its band(s) in place. In some cases, especially for larger or heavier centering cages 1301, two or more localization features can be beneficial.

In some cases, as shown in FIG. 13, the restraining tabs 1303 are on the same plane. In some cases, the restraining tabs 1303 can be anywhere along the circumference of the shaft. In some cases, the arc radius of the circumference between the restraining tabs 1303 can be from about 0 degrees and about 360 degrees. In some cases, the arc radius of the circumference between the restraining tabs 1303 can be from about 0 degrees to about 60 degrees, about 0 degrees to about 120 degrees, about 0 degrees to about 180 degrees, about 0 degrees to about 240 degrees, about 0 degrees to about 300 degrees, about 0 degrees to about 360 degrees, about 60 degrees to about 120 degrees, about 60 degrees to about 180 degrees, about 60 degrees to about 240 degrees, about 60 degrees to about 300 degrees, about 60 degrees to about 360 degrees, about 120 degrees to about 180 degrees, about 120 degrees to about 240 degrees, about 120 degrees to about 300 degrees, about 120 degrees to about 360 degrees, about 180 degrees to about 240 degrees, about 180 degrees to about 300 degrees, about 180 degrees to about 360 degrees, about 240 degrees to about 300 degrees, about 240 degrees to about 360 degrees, or from about 300 degrees to about 360 degrees. In some cases, the arc radius of the circumference between the restraining tabs 1303 can be at least about 10 degrees, at least about 20 degrees, at least about 30 degrees, at least about 40 degrees, at least about 50 degrees, at least about 60 degrees, at least about 70 degrees, at least about 80 degrees, at least about 90 degrees, at least about 100 degrees, at least about 110 degrees, at least about 120 degrees, at least about 130 degrees, at least about 140 degrees, at least about 150 degrees, at least about 160 degrees, at least about 170 degrees, at least about 180 degrees, at least about 190 degrees, at least about 200 degrees, at least about 210 degrees, at least about 220 degrees, at least about 230 degrees, at least about 240 degrees, at least about 250 degrees, at least about 260 degrees, at least about 270 degrees, at least about 280 degrees, at least about 290 degrees, at least about 300 degrees, at least about 310 degrees, at least about 320 degrees, at least about 330 degrees, at least about 340 degrees, at least about 350 degrees, or at least about 360 degrees. In some cases, the arc radius of the circumference between the restraining tabs 1303 can be at most about 10 degrees, at most about 20 degrees, at most about 30 degrees, at most about 40 degrees, at most about 50 degrees, at most about 60 degrees, at most about 70 degrees, at most about 80 degrees, at most about 90 degrees, at most about 100 degrees, at most about 110 degrees, at most about 120 degrees, at most about 130 degrees, at most about 140 degrees, at most about 150 degrees, at most about 160 degrees, at most about 170 degrees, at most about 180 degrees, at most about 190 degrees, at most about 200 degrees, at most about 210 degrees, at most about 220 degrees, at most about 230 degrees, at most about 240 degrees, at most about 250 degrees, at most about 260 degrees, at most about 270 degrees, at most about 280 degrees, at most about 290 degrees, at most about 300 degrees, at most about 310 degrees, at most about 320 degrees, at most about 330 degrees, at most about 340 degrees, at most about 350 degrees, or at most about 360 degrees.

FIG. 14 shows a perspective view of multiple centering cages on the distal end of a device 1400 described herein in accordance with embodiments described herein. Device 1400 comprises proximal centering cage 1401, distal centering cage 1403, and tip 1402. Device 1400 can comprise any of the devices described herein along with the dimensions of those devices. Tip 1402 can comprise any of the tips described herein. In some cases, the dual centering cages can be used in the presence of an inner and outer shaft, wherein proximal centering cage 1401 is attached to the outer shaft and the distal centering cage 1403 is on the inner shaft. In some cases, the proximal centering cage 1401 can be attached to a retaining ring. In some embodiments, the distal centering cage 1403 can either be attached to a retaining ring or be free floating, wherein the barriers determining the location of free floating can comprise the tip and the outer shaft. There may be two centering cages. In some cases, there are more than two centering cages. In some cases, each centering cage has the same number of centering loops. In some embodiments, various centering cages have varying numbers of centering loops, wherein the number can be 1, 2, 3, 4, 5, or more. The centering cage diameter may be the same across the centering cages. The diameter can be based off of the function of the device, and can be from about 1 and about 4 mm as discussed above. The centering cage diameter can be greater in more proximal centering cages, such that the diameter is the smallest in the distal centering cage 1403. A small centering cage can be useful in navigating angles, branch points, and smaller vessels. Additionally, the inner shaft and distal centering cage 1402 can be telescoped distal to the rest of the device.

The inner shaft can also have a smaller diameter, improving the navigation abilities of this design. In some cases, the inner shaft can have a diameter of about 1.1 mm. In some cases, the inner shaft can have a diameter of from about 0.5 mm and 1.5 mm. In some cases, the inner shaft can have a diameter of from about 0.5 mm to about 0.7 mm, about 0.5 mm to about 0.9 mm, about 0.5 mm to about 1.1 mm, about 0.5 mm to about 1.3 mm, about 0.5 mm to about 1.5 mm, about 0.7 mm to about 0.9 mm, about 0.7 mm to about 1.1 mm, about 0.7 mm to about 1.3 mm, about 0.7 mm to about 1.5 mm, about 0.9 mm to about 1.1 mm, about 0.9 mm to about 1.3 mm, about 0.9 mm to about 1.5 mm, about 1.1 mm to about 1.3 mm, about 1.1 mm to about 1.5 mm, or from about 1.3 mm to about 1.5 mm. In some cases, the inner shaft can have a diameter less than about 0.5 mm, less than about 0.7 mm, less than about 0.9 mm, less than about 1.1 mm, less than about 1.3 mm, and finally, or less than about 1.5 mm. In some cases, the inner shaft can have a diameter greater than about 0.5 mm, greater than about 0.7 mm, greater than about 0.9 mm, greater than about 1.1 mm, greater than about 1.3 mm, and finally, or greater than about 1.5 mm.

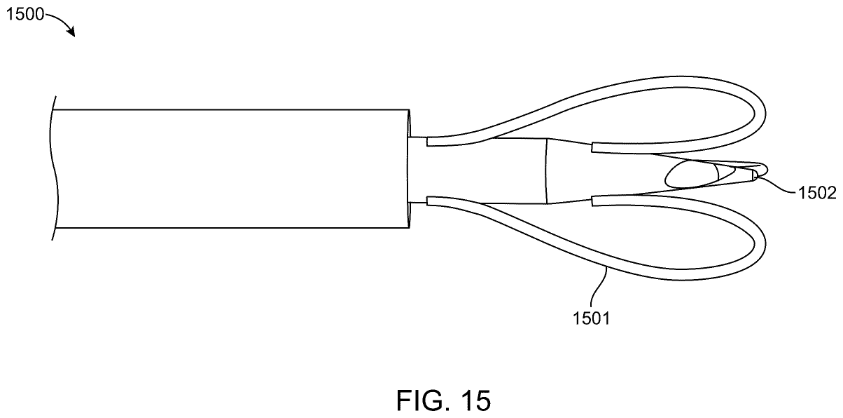

FIG. 15 shows a side view of an alternate centering cage on the distal end of a device 1500 described herein in accordance with embodiments described herein. Device 1500 comprises centering cage 1501 and tip 1502. Device 1500 can comprise any of the devices described herein along with the dimensions of those devices. Tip 1502 can comprise any of the tips described herein. In some cases, the loops of the centering cage 1501 extend beyond the tip. This can improve centering while crossing an occlusion. This can also protect the vessel wall from inadvertent injury by a sharp tip 1502. The tip 1502 can be sharp enough to penetrate an occlusion's plaque and/or occlusion mass. When axial force is applied to the inner shaft, or only shaft in the cases of devices where there is only one shaft, the centering cage 1501 loops may increase in width. The centering cage 1501 loops can then retract such that the tip extends beyond the centering cage 1501 loops and may apply force at and around the central axis of the device. Without axial force, the tip 1502 is in a neutral state within the confines of the centering cage 1501 loops. Unlike some of the other designs, the distal end of the centering cage 1501 loops is directly attached to the tip 1502, while the proximal end of the centering cage 1501 loops is directly attached to the inner shaft, or only shaft in the cases of devices where there is only one shaft.

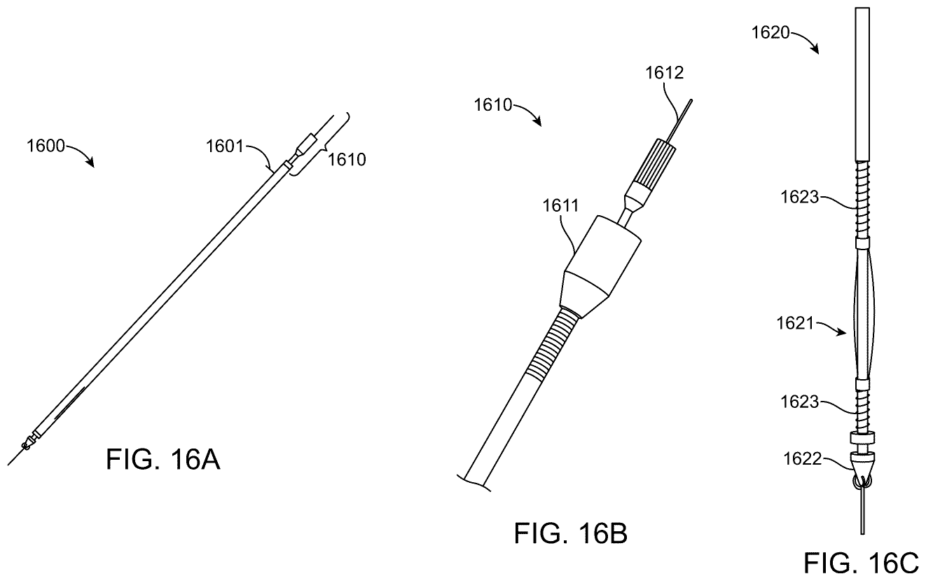

FIGS. 16A-16C show a perspective view, a magnified perspective view of the distal end, and an inner side view, respectively, of an alternate centering cage of a device 1600 described herein in accordance with embodiments described herein. Device 1600 can comprise an actuation lever 1601, a distal end 1610, and a spring-actuated retractable centering system 1620. With reference to FIG. 16B, distal end 1610 can comprise a guidewire 1612 and an actuator knob 1611. With reference to FIG. 16C, spring-actuated retractable centering system 1620 can comprise spring actuation mechanism 1623, tip 1622, and centering cage 1621. The spring actuation mechanism 1623 can have one or more sections underlying one or more actuation mechanisms. In FIGS. 16A-16C, the two sections spring actuation mechanism 1623 in FIG. 16C underlie the actuation lever in FIG. 16A and actuation knob in FIG. 16B. Various actuation locations and mechanisms of actuations can be used. Other features of device 1600, such as the number of shafts, the proximal end, and the dimensions, can comprise any of the devices described herein along with the dimensions of those devices. Tip 1622 can comprise any of the tips described herein.

Device 1600 comprises a spring actuated retractable centering system and centering cage. The centering system diameter can be adjusted from about 0 mm to about 3 mm while crossing the occlusion. The diameter can be manually adjusted. In some cases, the diameter can be adjusted from about 0 mm to about 0.5 mm, about 0 mm to about 1 mm, about 0 mm to about 1.5 mm, about 0 mm to about 2 mm, about 0 mm to about 2.5 mm, about 0 mm to about 3 mm, about 0.5 mm to about 1 mm, about 0.5 mm to about 1.5 mm, about 0.5 mm to about 2 mm, about 0.5 mm to about 2.5 mm, about 0.5 mm to about 3 mm, about 1 mm to about 1.5 mm, about 1 mm to about 2 mm, about 1 mm to about 2.5 mm, about 1 mm to about 3 mm, about 1.5 mm to about 2 mm, about 1.5 mm to about 2.5 mm, about 1.5 mm to about 3 mm, about 2 mm to about 2.5 mm, about 2 mm to about 3 mm, or from about 2.5 mm to about 3 mm. In some cases, the diameter can be adjusted to greater than about 0 mm, greater than about 0.5 mm, greater than about 1 mm, greater than about 1.5 mm, greater than about 2 mm, greater than about 2.5 mm, or greater than about 3 mm. In some cases, the diameter can be adjusted to less than about 0.5 mm, less than about 1 mm, less than about 1.5 mm, less than about 2 mm, less than about 2.5 mm, or less than about 3 mm.

Device 1600 can be beneficial in long occlusions in vessels with size variations occurring in the same regions as the occlusion. A user can adjust the diameter of device 1600 as useful depending on the diameter of the vessel. Similarly, device 1600 can be beneficial in multi-segment occlusions in vessels of different diameters. Device 1600 can also be helpful in more calcified lesions. Fully retracting the centering cage minimizes the device diameter and thus reduces the drag of the device.

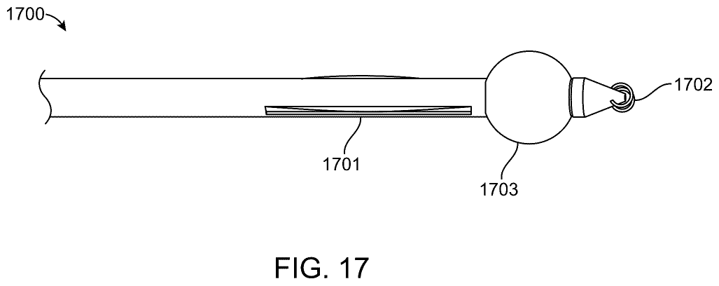

FIG. 17 shows a side view of an expandable balloon-assisted centering cage on the distal end of a device 1700 described herein in accordance with embodiments described herein. Device 1700 comprises centering cage 1701, tip 1702, and balloon 1703. Device 1700 can comprise any of the devices described herein along with the dimensions of those devices. Tip 1702 can comprise any of the tips described herein. In some cases, balloon 1703 can be coated with one or more medications. Balloon 1703 can assist with coating vessels with one or more medications. In crossing a harder and more calcified lesion, a user may remove a device as described herein and insert an angioplasty balloon 1703 to dilate and break the plaque and/or occlusion mass. The user can then reinsert the original device to continue crossing. In some cases, by having a balloon 1703 on the device that crosses the occlusion, a user can save time and effort by using this embodiment with in-built balloon. In some cases, the balloon 1703 can be deflated upon insertion but can be inflated upon encountering a highly calcified lesion. In some cases, the balloon 1703 can be inflated prior to insertion of the balloon. In some cases, the tip 1702 can be inserted into the lesion, the balloon can follow it, and the balloon can inflate within the lesion to break it down from inside. Either way, the balloon can be used to allow the remainder of the device to cross the occlusion.

Balloon 1703 can assist with pre-dilating moderately hard occlusion prior to further advancement by the centering system. Accordingly, balloon 1703 has a greater diameter than the centering system. In some cases, for example when the centering system is static and cannot be made smaller, the diameter of balloon 1703 is greater than the static diameter. In some cases, for example when the centering system is retractable or otherwise minimizable, the diameter of balloon 1703 can be smaller than, equal to, or greater than the maximal diameter of the centering system while still being greater than the minimal diameter of the centering system.

In some cases, the balloon may comprise an elastic material. In some cases, the balloon may comprise inelastic material. In some cases, the inelastic balloon material may help to minimize lateral pressure onto surrounding vessel walls. The balloon may compress the plaque and/or occlusion mass against the walls while minimally stretching the vessel wall, or not stretching it at all. In some cases, the inelastic material may minimize overexpansion of the balloon. The balloon may be deployed at a low pressure. In some cases, the pressure of the balloon is sufficient to dilate an occlusion for the rest of the device to proceed through the occlusion without exerting significant pressure on the vessel wall, such that the vessel wall is not extended or injured.

In some cases, the diameter of the balloon can be from about 2 mm and about 4 mm. In some cases, the diameter of the balloon can be from about 1 mm and about 5 mm. The diameter can be from about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 1.5 mm to about 4.5 mm, about 1.5 mm to about 5 mm, about 2 mm to about 2.5 mm, about 2 mm to about 3 mm, about 2 mm to about 3.5 mm, about 2 mm to about 4 mm, about 2 mm to about 4.5 mm, about 2 mm to about 5 mm, about 2.5 mm to about 3 mm, about 2.5 mm to about 3.5 mm, about 2.5 mm to about 4 mm, about 2.5 mm to about 4.5 mm, about 2.5 mm to about 5 mm, about 3 mm to about 3.5 mm, about 3 mm to about 4 mm, about 3 mm to about 4.5 mm, about 3 mm to about 5 mm, about 3.5 mm to about 4 mm, about 3.5 mm to about 4.5 mm, about 3.5 mm to about 5 mm, about 4 mm to about 4.5 mm, about 4 mm to about 5 mm, or from about 4.5 mm to about 5 mm. The diameter can be less than about 1 mm, less than about 1.5 mm, less than about 2 mm, less than about 2.5 mm, less than about 3 mm, less than about 3.5 mm, less than about 4 mm, less than about 4.5 mm, or less than about 5 mm. The diameter can be greater than about 1 mm, greater than about 1.5 mm, greater than about 2 mm, greater than about 2.5 mm, greater than about 3 mm, greater than about 3.5 mm, greater than about 4 mm, greater than about 4.5 mm, or greater than about 5 mm.

### Related Devices and Accessories

Described herein are related devices and accessories to the devices described throughout the application. In some cases, the devices comprise embolic protection devices. In some embodiments, the devices comprise microcatheters. In some cases, the devices comprise devices similar to those described throughout the application but with additional guidewire lumens and/or exits.

Provided herein are devices for atheroplasty, a novel concept of tunneling through occlusions similar to an atherectomy but without scraping or removal of the plaque and/or occlusion mass buildup, thereby making them safer than an atherectomy. In some embodiments, atheroplasty devices do not expand or stretch the vessel wall. The vessels can comprise coronary arteries, carotid arteries, iliac arteries and veins, femoral arteries and veins, popliteal arteries and veins, radial arteries, and other arteries and veins throughout the body. In some cases, the devices comprise a centering element that keeps them centered in the vessel, thereby significantly decreasing the chance of perforation or dissection of the vessel wall. In some cases, the devices are small, non-invasive devices that can be made relatively cheaply.

Described herein are accessories to the devices and methods described herein. In some embodiments, the accessories comprise stylets for steering devices and/or tips of devices once inserted in a blood vessel. In some cases, the accessories comprise specially formed guidewires.

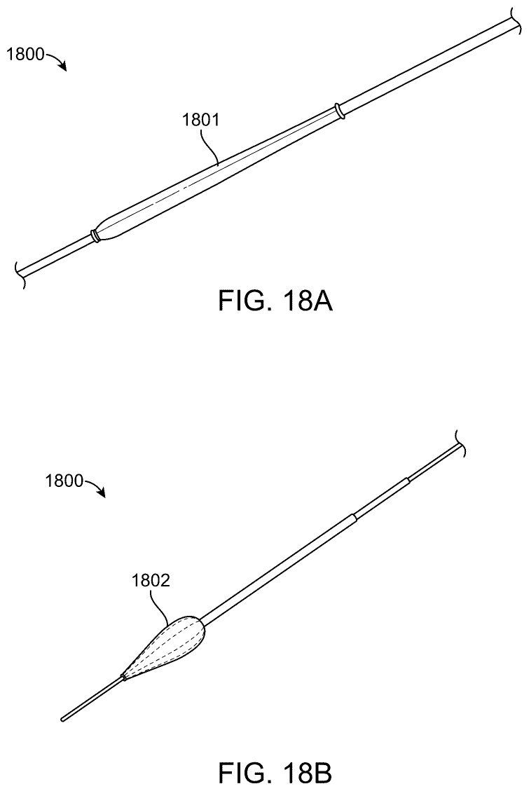

FIGS. 18A-18B show relaxed and expanded, respectively, perspective views of a distal end of a device 1800 described throughout the application in accordance with embodiments described throughout the application. Device 1800 comprises a bulbous distal end. In some cases, with reference to FIG. 18A, the distal end is in its collapsed or relaxed, smaller form 1801. In its smaller form 1801, the diameter of the distal end can be from about 1.5 mm to about 2.2 mm. In its smaller form 1801, the diameter of the distal end can be from about 1.5 mm to about 1.8 mm, about 1.5 mm to about 2 mm, about 1.5 mm to about 2.2 mm, about 1.8 mm to about 2 mm, about 1.8 mm to about 2.2 mm, or from about 2 mm to about 2.2 mm. In its smaller form 1801, the diameter of the distal end can be less than about 1.5 mm, less than about 1.6 mm, less than about 1.7 mm, less than about 1.8 mm, less than about 1.9 mm, less than about 2.0 mm, less than about 2.1 mm, or less than about 2.2 mm. In its smaller form 1801, the diameter of the distal end can be greater than about 1.5 mm, greater than about 1.6 mm, greater than about 1.7 mm, greater than about 1.8 mm, greater than about 1.9 mm, greater than about 2.0 mm, greater than about 2.1 mm, or greater than about 2.2 mm.

In some cases, with reference to FIG. 18B, the distal end is in its actuated, larger form 1802. In its larger form 1802, the largest diameter of the distal end can be from about 3 mm to about 7 mm. In its larger form 1802, the diameter of the distal end can be from about 3 mm to about 4 mm, about 3 mm to about 5 mm, about 3 mm to about 6 mm, about 3 mm to about 7 mm, about 4 mm to about 5 mm, about 4 mm to about 6 mm, about 4 mm to about 7 mm, about 5 mm to about 6 mm, about 5 mm to about 7 mm, or from about 6 mm to about 7 mm. In its larger form 1802, the diameter of the distal end can be less than about 3 mm, less than about 4 mm, less than about 5 mm, less than about 6 mm, or less than about 7 mm. In its larger form 1802, the diameter of the distal end can be greater than about 3 mm, greater than about 4 mm, greater than about 5 mm, greater than about 6 mm, or greater than about 7 mm.

When active, the bulbous distal end can be comprised of petal-shaped structures. The petal-shaped structures can comprise nitinol or other similar shape memory alloys and materials. When not actuated or active, the petal structure can become smaller, creating a smaller size tube over the shaft. Device 1800 can comprise any of the devices described throughout the application along with the dimensions of those devices.

Actuating distal end 1801 into distal end 1802 either before or during passage through the occlusion can exert pressure on plaque and/or occlusion mass as its tip drills through. Due to the dottering effect, the plaque and/or occlusion mass can be compressed similar to a snow plough. As the device 1800 proceeds through the occlusion, distal end 1802 can help to compact the plaque and/or occlusion mass along the walls of the vessel. In some cases, device 1800 can distal end 1802 can do this without extending, expanding, or stretching the vessel wall. While passing through an occlusion, the distal end 1802 can exert a minimal pressure on the surrounding plaque and/or occlusion mass and, through the plaque and/or occlusion mass, on the walls of the vessel, of less than about 2 mm Hg. The distal end 1802 can exert a pressure of less than about 1 mm Hg. The distal end 1802 can exert a pressure of from about 1 mm Hg to about 2 mm Hg. In some cases, the pressure exerted may be from about 2 mm Hg to about 10 mm Hg. In some cases, the pressure exerted may be from about 2 mm Hg to about 4 mm Hg, about 2 mm Hg to about 6 mm Hg, about 2 mm Hg to about 8 mm Hg, about 2 mm Hg to about 10 mm Hg, about 4 mm Hg to about 6 mm Hg, about 4 mm Hg to about 8 mm Hg, about 4 mm Hg to about 10 mm Hg, about 6 mm Hg to about 8 mm Hg, about 6 mm Hg to about 10 mm Hg, or from about 8 mm Hg to about 10 mm Hg. In some cases, there is minimal or no stretching of the vessel walls. In some cases, due to this low pressure, even though the device compresses the pulverized plaque and/or occlusion mass against the sides of the vessel, it does not cause expansion of the vessel wall. Accordingly, when passing through the blood vessel (e.g., the clean and/or non-occluded parts of the vessel) on its way to an occlusion mass, the devices described herein likewise do not cause expansion, widening, extension, stretching, or any combination thereof. Barotrauma, or pressure-based injury, to vessel walls can occur due to a high strain rate. As such, stretching the vessel wall slowly may not cause as much damage, if any. The devices described herein may have a strain rate of zero or one that is negligible to vessel walls in non-balloon variations due to lack of lateral force application and thus no active application of lateral strain rate. As such, there may be no vessel dilation or injury.

In some cases, in its active form, the distal end 1802 can be coated with one or more antiproliferative medications. Distal end 1802 can assist with coating vessels with one or more medications. In some cases, by having distal end 1802 on the device that crosses the occlusion, a user can save time and effort associated with withdrawing the device and inserting a second device coated with antiproliferative medications.

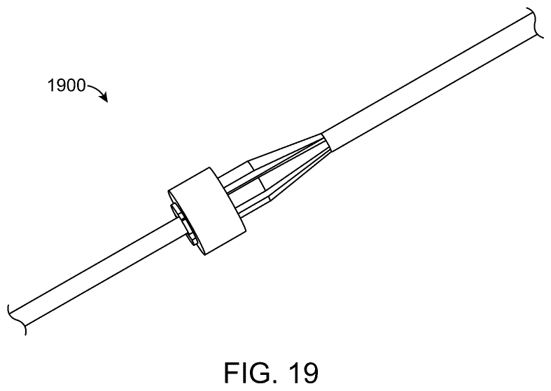

FIG. 19 shows a perspective view of an example actuator 1900 of the balloon of FIGS. 18A-18B on the proximal end of a device 1800 described throughout the application in accordance with embodiments described throughout the application. The actuator can be spring-based, manual, or any other type of actuator.

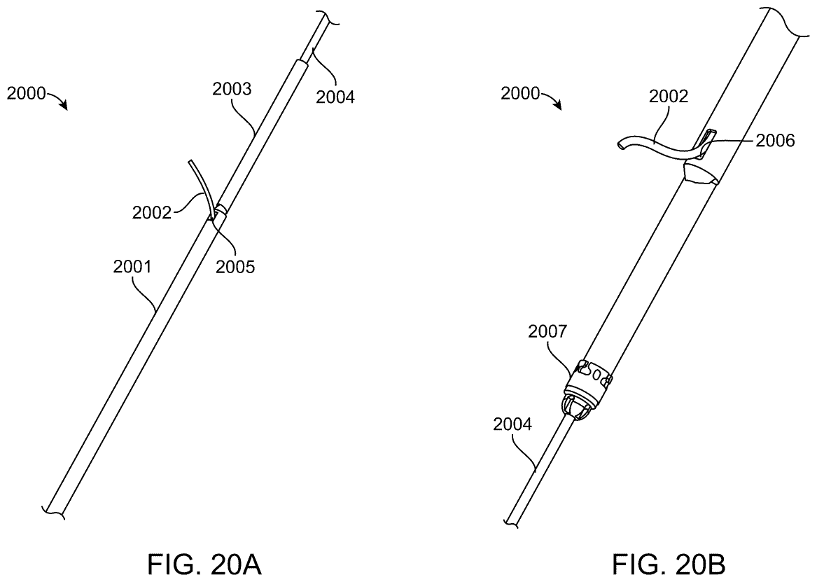

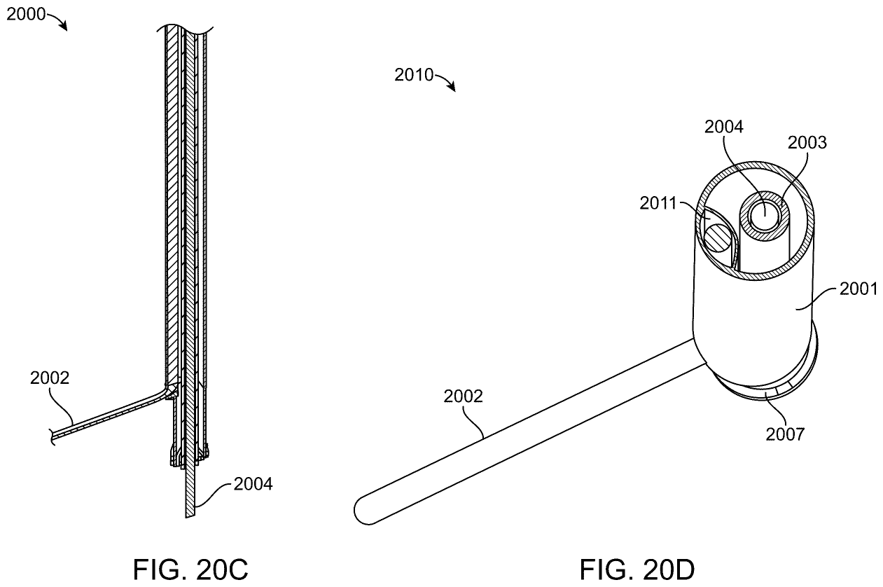

FIGS. 20A-D show perspective views (A, B, and D) and a side view (C) of a dual-lumen device. FIG. 20A shows a perspective view of a proximal end of a dual-lumen device 2000 as described throughout the application in accordance with embodiments described throughout the application. Device 2000 can comprise outer shaft 2001, second guidewire 2002, inner shaft 2003, first guidewire 2004, and entrance port 2005 for second guidewire 2002. With reference to FIG. 20B, device 2000 further comprises distal exit port 2006 and tip 2007 on the distal end of the device. FIG. 20C shows a side view of the inside of the dual-lumen device 2000 at the junction of two guidewires 2002 and 2004. FIG. 20D shows a perspective top view of the inside 2010 of the dual lumen. Inside 2010 can comprise outer shaft 2001, second guidewire 2002, inner shaft 2003, first guidewire 2004, tip 2007, and channel for second guidewire 2011. The dual lumen device 2000 can comprise one or more guidewire lumens and one or more guidewire exit ports. The dual lumen device 2000 can comprise two or more guidewire lumens and two or more guidewire exit ports. One or more of the exit ports can point to the side of the device or towards the vessel lumen. Device 2000 can comprise any of the devices described throughout the application along with the dimensions of those devices. Tip 2007 can comprise any of the tips described throughout the application.

If the device 2000 is veering towards the vessel wall, the secondary guidewire 2002 can be advanced out of its exit port 2006. Exit port 2006 can be proximal to the tip 2007 and to the side of the device 2000. As the secondary guidewire 2002 has a separate channel 2011 from the primary channel or lumen, the secondary guidewire 2002 can be separately steered. Second guidewire 2002 can be separately steered towards the blood vessel's central axis, thereby helping to recenter the device 2000.

Once the secondary wire 2002 tip is located near or close to a proximal side of the central axis of the occlusion, device 2000 can be removed along with primary guidewire 2004. Device 2000, whether the same device or a sterile version of the device, may be reintroduced over the original secondary guidewire 2002. The original secondary guidewire 2002 can be located in the primary lumen of device 2000, thus becoming the primary guidewire 2004.

In some cases, the distance between the distal end of the tip and the distal end of the outer shaft is from about 0.5 cm to about 2.5 cm. In some cases, the distance is from about 0.5 cm to about 1 cm, about 0.5 cm to about 1.5 cm, about 0.5 cm to about 2 cm, about 0.5 cm to about 2.5 cm, about 1 cm to about 1.5 cm, about 1 cm to about 2 cm, about 1 cm to about 2.5 cm, about 1.5 cm to about 2 cm, about 1.5 cm to about 2.5 cm, or from about 2 cm to about 2.5 cm. In some cases, the distance is less than 0.5 cm, less than 1 cm, less than 1.5 cm, less than 2 cm, or less than 2.5 cm. In some cases, the distance is greater than 0.5 cm, greater than 1 cm, greater than 1.5 cm, greater than 2 cm, or greater than 2.5 cm.

Accordingly, the distance between the exit holes of the two guidewires can be from about 1 cm to about 3 cm. In some cases, the distance is from about 1 cm to about 1.5 cm, about 1 cm to about 2 cm, about 1 cm to about 2.5 cm, about 1 cm to about 3 cm, about 1.5 cm to about 2 cm, about 1.5 cm to about 2.5 cm, about 1.5 cm to about 3 cm, about 2 cm to about 2.5 cm, about 2 cm to about 3 cm, or from about 2.5 cm to about 3 cm. In some cases, the distance is less than 1 cm, less than 1.5 cm, less than 2 cm, less than 2.5 cm, or less than 3 cm. In some cases, the distance is greater than 1 cm, greater than 1.5 cm, greater than 2 cm, greater than 2.5 cm, or greater than 3 cm.

The benefit of the dual lumen device 2000 is that whenever the device tip inadvertently approaches the vessel wall or enters the subintimal space, the dual lumen can help redirect the device toward the central axis of the occlusion.

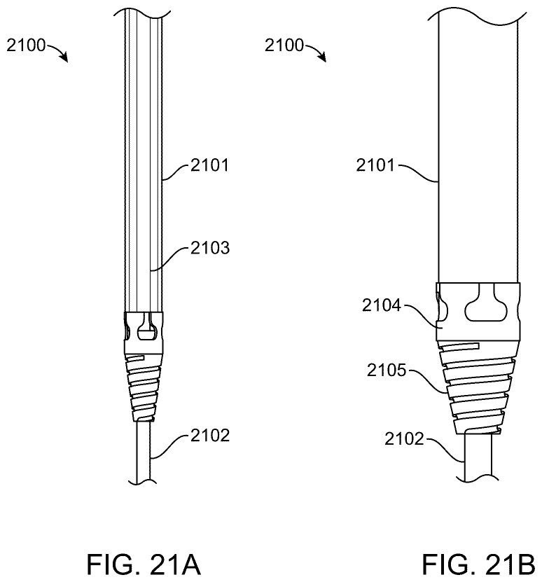

FIGS. 21A-21B show a side view and a magnified side view, respectively, of an embodiment of a microcatheter device 2100. Device 2100 can comprise outer shaft 2101, guidewire 2102, inner shaft 2103, retaining ring 2104, and tip 2105. This device 2100 can have a robust shaft. In some cases, the outer shaft 2101 can be made of a stainless steel braid for reinforced strengths. The stainless steel braid can provide a balance of flexibility, kink resistance, and pushability. In some cases, the inner shaft 2103 can comprise a stainless steel helical coil. This can similarly provide durability and resistance to deformation while allowing the device to be both flexible and strong. Furthermore, the device 2100 comprises a torpedo-shaped tip. The conical profile and helically cut surface of the tip 2105 can efficiently cut and slow-plough through fibrotic and calcified plaque and/or occlusion masses. These features can improve the robustness of the catheter to cross through otherwise “balloon uncrossable” occlusions and/or non-CTO stenoses. This can include 6-9% of coronary occlusions that may otherwise be difficult to cross with conventional balloons or microcatheters

In some cases, the inner shaft 2103 can rotate. The tip 2105 can rotate. Due to a lack of wide inhibitory features, rotating or torqueing the inner shaft 2103 and tip 2105 can be drag-free and fatigue-resistant.

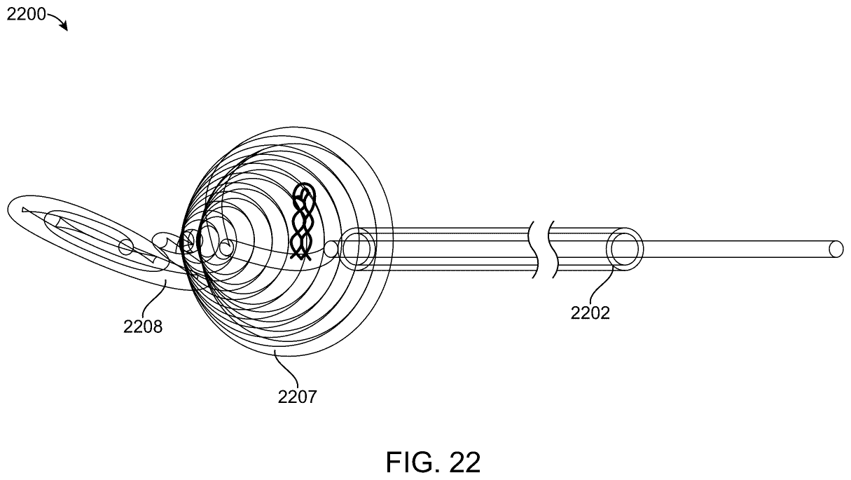

FIG. 22 shows a perspective top view of the proximal end of embolic protection device 2200. Embolic protection device 2200 comprises delivery catheter or interventional device 2202, filter cone 2207, and pig-tail tip 2208.

Atherosclerotic emboli can be encountered during percutaneous cardiovascular interventions such as during crossing of occlusion, balloon angioplasty, and atherectomy. Thromboembolism can also occur during interventional treatment of acute arterial and graft thrombosis. Percutaneous interventions for aorto-coronary saphenous vein graft (SVG) occlusions pose special risk for atheroembolizations due to the underlying soft occlusive material. Acute arterial or graft occlusions can also contain soft, easily friable thrombus. Athero or thromboembolization may be sometimes inconsequential, however these emboli may migrate distally and occlude smaller vascular bed and cut-off oxygenated blood flow, thereby risking in end-organ damage. This ischemic risk may be influenced by emboli size, baseline run-off status, status of collaterals, and acuity of presenting condition. Embolic damage can present with stroke, TIA, MI, toe ulcers, or acute limb ischemia depending on the blood vessel intervened.

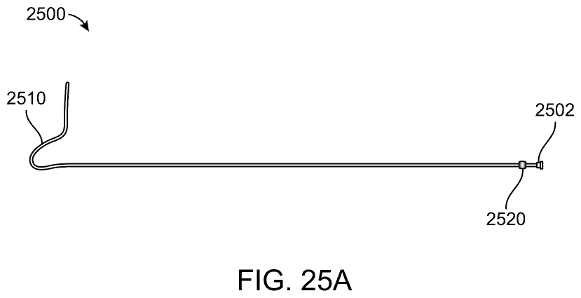

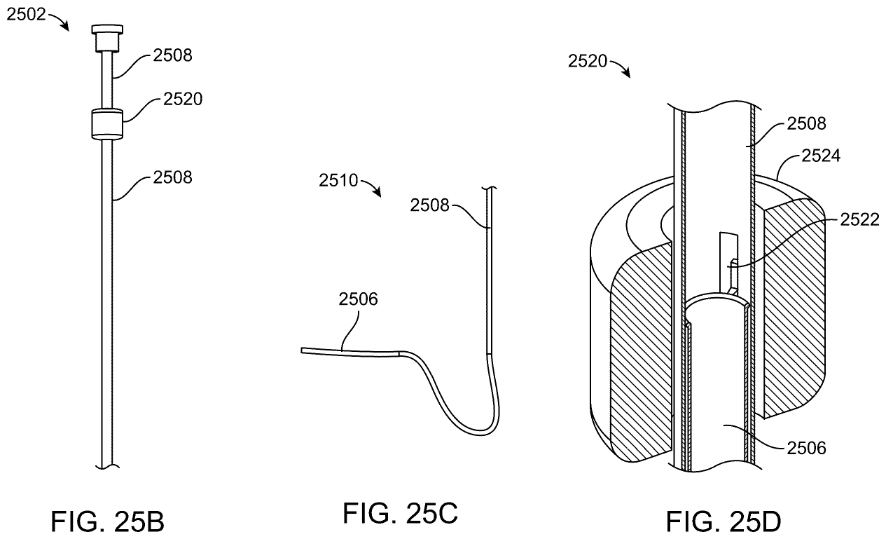

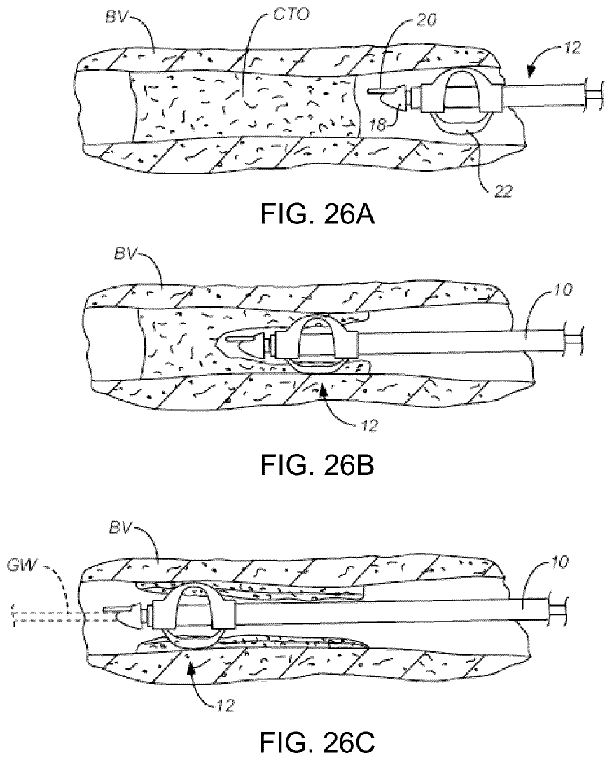

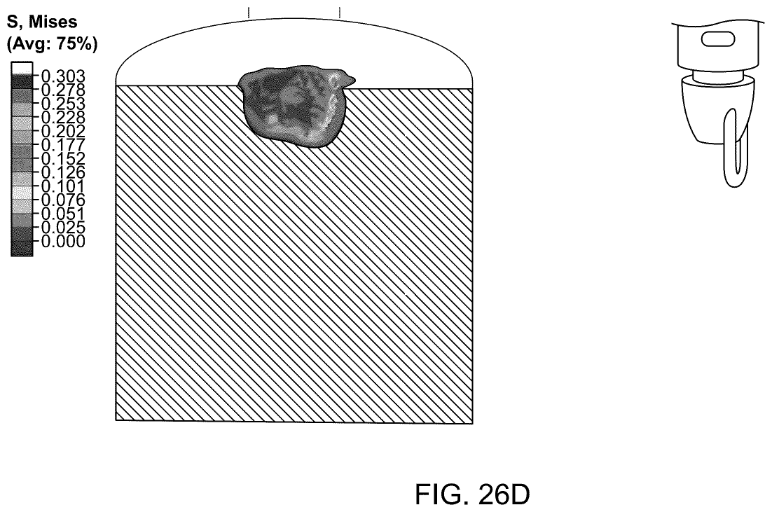

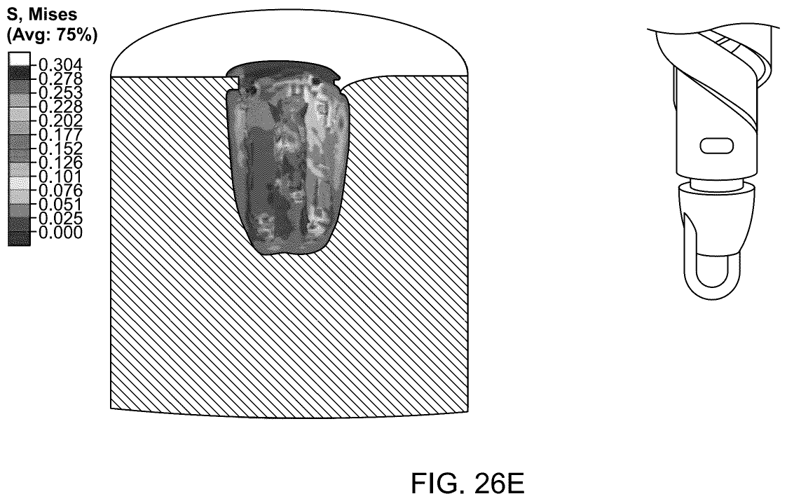

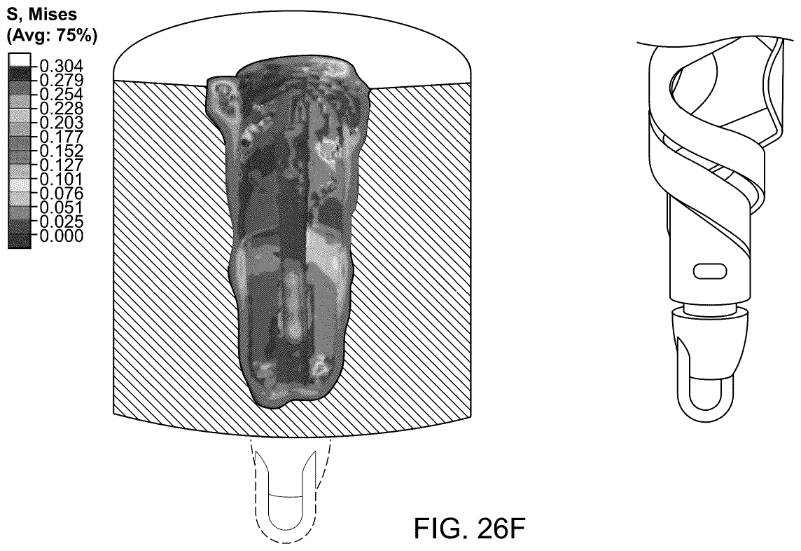

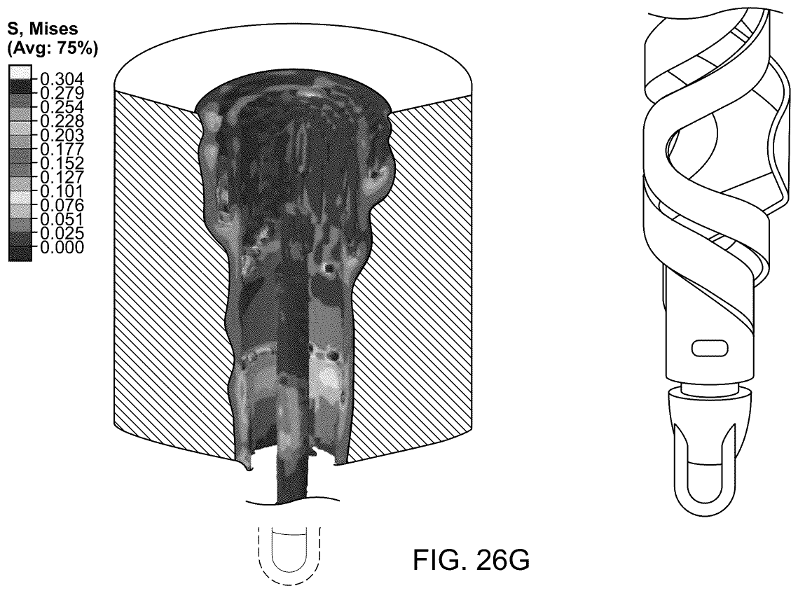

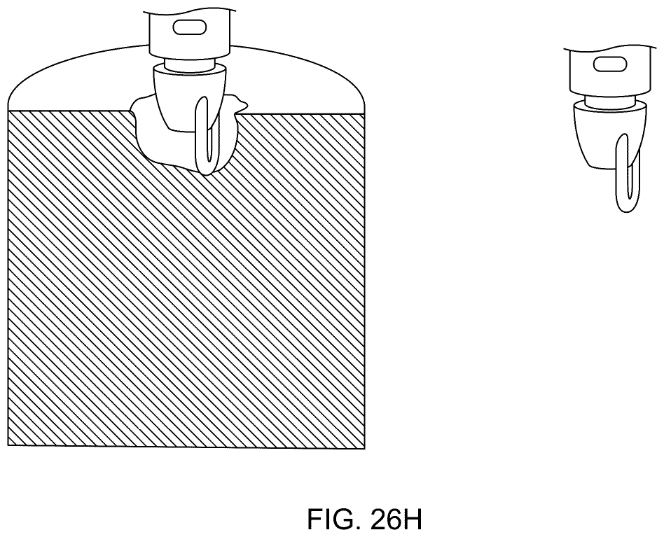

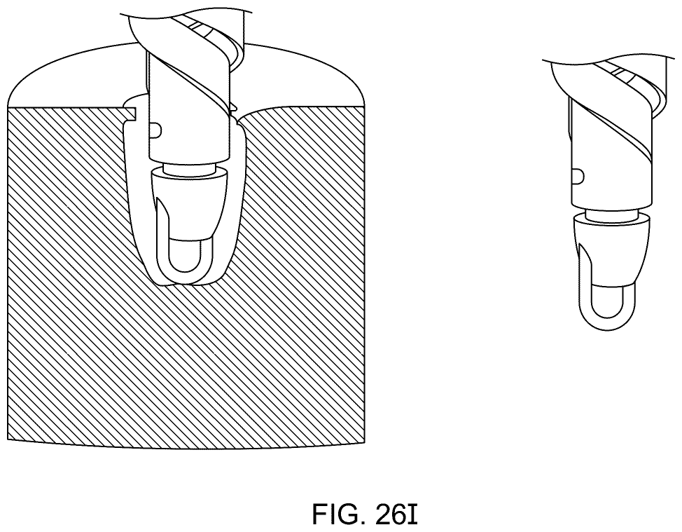

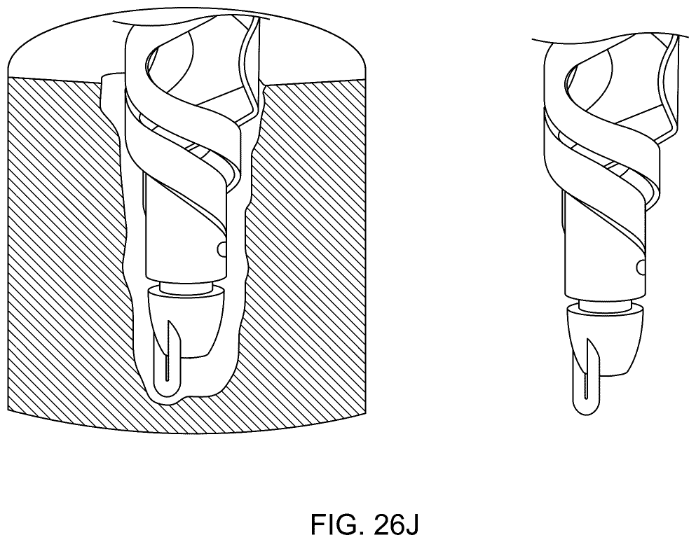

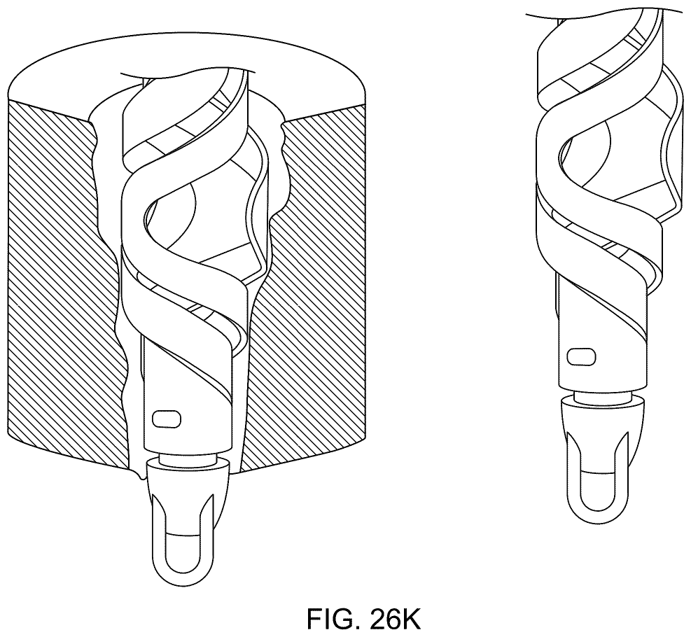

As further discussed below with regards to FIGS. 25A-C and FIGS. 26A-D, chronic total occlusions (CTO) generally associated with large plaque and/or occlusion mass burden hence may pose high risk for embolization during their recanalyzations. It may be difficult to extract thrombus or atheroma debris from downstream smaller vessels either with percutaneous or open surgical methods. In some cases, a CTO needs to be crossed with a guidewire or CTO crossing device before either a filter- or balloon-based embolic protection device (EPD) can be placed distal to the occlusion in the distal lumen. However, this crossing process of CTO itself poses embolic risk, particularly while crossing the distal cap using a large profile crossing device (usually 6 F or 7 F).

Due to its unique properties such as shape memory, superelasticity, radiopacity and biocompatibility, EPD 2200 may comprise nitinol. In some cases, EPD 2200 can have a constrained state. In its constrained state, EPD 2200 can be a straight wire. In some cases, EPD 2200 can be a straight Nitinol wire. EPD 2200 can have a diameter from about 0.014″ to about 0.018″. EPD 2200 can have a length from about 180 cm to about 300 cm. In this constrained form EPD 2200 can be easily disposed within the central guidewire lumen of a balloon/stent catheters, CTO crossing, and/or atherectomy devices.

In some cases, EPD wire 2200 can have a diameter from about 0.01″ and 0.02″ in its constrained state. In some cases, EPD wire 2200 can have a diameter from about 0.01″ to about 0.012″, about 0.01″ to about 0.014″, about 0.01″ to about 0.016″, about 0.01″ to about 0.018″, about 0.01″ to about 0.02″, about 0.012″ to about 0.014″, about 0.012″ to about 0.016″, about 0.012″ to about 0.018″, about 0.012″ to about 0.02″, about 0.014″ to about 0.016″, about 0.014″ to about 0.018″, about 0.014″ to about 0.02″, about 0.016″ to about 0.018″, about 0.016″ to about 0.02″, or from about 0.018″ to about 0.02″. In some cases, the diameter can be less than about 0.01″, less than about 0.012″, less than about 0.014″, less than about 0.016″, less than about 0.018″, or less than about 0.02″. In some cases, the diameter can be greater than about 0.01″, greater than about 0.012″, greater than about 0.014″, greater than about 0.016″, greater than about 0.018″, or greater than about 0.02″.

In some cases, EPD wire 2200 can have a length from about 150 cm to 350 cm in its constrained state. In some cases, EPD wire 2200 can have a length from about 150 cm to about 200 cm, about 150 cm to about 250 cm, about 150 cm to about 300 cm, about 150 cm to about 350 cm, about 200 cm to about 250 cm, about 200 cm to about 300 cm, about 200 cm to about 350 cm, about 250 cm to about 300 cm, about 250 cm to about 350 cm, or from about 300 cm to about 350 cm. In some cases, EPD wire 2200 can have a length less than about 150 cm, less than about 170 cm, less than about 190 cm, less than about 210 cm, less than about 230 cm, less than about 250 cm, less than about 270 cm, less than about 290 cm, less than about 310 cm, less than about 330 cm, less than about 350 cm. In some cases, EPD wire 2200 can have a length greater than about 150 cm, greater than about 170 cm, greater than about 190 cm, greater than about 210 cm, greater than about 230 cm, greater than about 250 cm, greater than about 270 cm, greater than about 290 cm, greater than about 310 cm, greater than about 330 cm, greater than about 350 cm.

Nitinol is a metal alloy that can be made up of Nicker and titanium. Nitinol can have two crystalline phases: a weaker, low temperature phase (masternsite) and a stronger, high temperature phase (austenite); which are mutually reversible. In some embodiments, from about 15 cm to about 20 cm of the distal segment of the EPD 2200, starting about 5 cm proximal to the distal end, is tightly wrapped around a cone shaped mold. The mold can measure from about 7 mm to about 10 mm in the widest diameter and from about 10 mm to about 15 mm in height. The outer surface of the mold may be threaded at a diameter of from about 0.014″ to about 0.018″, in which the device/wire coils can be parked for heat treatment. The space in between the threads (hence wire loops) can be configured to be not more than from about 40 microns to about 50 microns. This may give a shape memory to the device with a less than about 50 microns inter-coil gap in its fully programmed state which can only allow emboli less than 50 microns in size and red blood cells (less than 10 microns in diameter) to flow through the coils. In some cases, emboli less than about 50 microns are found to be clinically inconsequential. As such, this design can avoid obstructing blood flow while still capturing debris. The cone mold also has 2 wire constrainers: one on the top surface and a second one over the inner surface, which can allow wire bending and device configuration stabilization during heat-treatment. The distal-most 5 cm wire segment can also be wound inside a pig-tail shape threading on the surface of another mold. This pig-tail tip configuration 2208 of the distal end of the device/wire can be used to safeguard the blood vessel wall from injury from otherwise a sharp and pointed distal tip.

In this stage, the device/wire can resemble a filter-cone 2207 measuring from about 7 mm to about 10 mm at its widest (at upper border) diameter, from about 10 mm to about 15 mm in height, and centrally disposed in the blood vessel lumen with its periphery in flush with the blood vessel wall. In some cases, the widest diameter can be from about 5 mm to about 15 mm. In some cases, the widest diameter can be from about 5 mm to about 7 mm, about 5 mm to about 9 mm, about 5 mm to about 11 mm, about 5 mm to about 13 mm, about 5 mm to about 15 mm, about 7 mm to about 9 mm, about 7 mm to about 11 mm, about 7 mm to about 13 mm, about 7 mm to about 15 mm, about 9 mm to about 11 mm, about 9 mm to about 13 mm, about 9 mm to about 15 mm, about 11 mm to about 13 mm, about 11 mm to about 15 mm, or from about 13 mm to about 15 mm. The widest diameter can be less than about 5 mm, less than about 7 mm, less than about 9 mm, less than about 11 mm, less than about 13 mm, or less than about 15 mm. The widest diameter can be greater than about 5 mm, greater than about 7 mm, greater than about 9 mm, greater than about 11 mm, greater than about 13 mm, or greater than about 15 mm. In some cases, the height of the filter cone 2207 can be from about 5 mm to about 20 mm. In some cases, the height of the filter cone 2207 can be from about 5 mm to about 8 mm, about 5 mm to about 11 mm, about 5 mm to about 14 mm, about 5 mm to about 17 mm, about 5 mm to about 20 mm, about 8 mm to about 11 mm, about 8 mm to about 14 mm, about 8 mm to about 17 mm, about 8 mm to about 20 mm, about 11 mm to about 14 mm, about 11 mm to about 17 mm, about 11 mm to about 20 mm, about 14 mm to about 17 mm, about 14 mm to about 20 mm, or from about 17 mm to about 20 mm. The height can be less than about 5 mm, less than about 8 mm, less than about 11 mm, less than about 14 mm, less than about 17 mm, or less than about 20 mm. The height can be greater than about 5 mm, greater than about 8 mm, greater than about 11 mm, greater than about 14 mm, greater than about 17 mm, or greater than about 20 mm.

The space in between the wire loops can be pre-configured to be about less than 50 microns to capture and protect the vascular bed from any emboli greater than or equal to 50 microns and at the same time allow uninterrupted blood flow across the filter during intervention. In some cases, the space in between loops can be less than about 20 microns, less than about 30 microns, less than about 40 microns, or less than about 50 microns. In some cases, the space in between loops can be greater than about 10 microns, greater than about 20 microns, greater than about 30 microns, or greater than about 40 microns.

The distal 5 cm segment of the device/wire can attain its austenite phase shape i.e. “pigtail” configuration 2208. The diameter of this pigtail end configuration 2208 can be pre-programmed the heat treatment process to be at least from about 1 mm to about 2 mm larger than the target vessel in which it is to be parked, which is to prevent any inadvertent movement, migration, or uncoiling of the device during intervention that can lead to failure of its embolic protection function.

The segment of device/wire 2200 immediately proximal to the filter-cone 2207 is preshaped to bend inside along the inner surface of the cone and at the tip of the cone takes an upward turn to become straight wire. From this point the device/wire 2200 is an untreated segment and functions as any other guidewire and can be used for catheter/device exchange during intervention.

At the completion of intervention, the therapeutic device can be co-axially exchanged out over the device/wire 2200, with from about a 6 F to about a 7 F multipurpose or similarly shaped guide catheter, whose tip is carefully advanced close to the tip of the filter-cone under fluoroscopic guidance. An aspiration syringe from about 30 cc to about 60 cc can be attached to the distal end of the guide catheter and the collected debris from the cone filter can be aspirated with or without saline flush. The aspiration syringe can be from about 10 cc to about 80 cc. The aspiration syringe can be from about 10 cc to about 20 cc, about 10 cc to about 30 cc, about 10 cc to about 40 cc, about 10 cc to about 50 cc, about 10 cc to about 60 cc, about 10 cc to about 70 cc, about 10 cc to about 80 cc, about 20 cc to about 30 cc, about 20 cc to about 40 cc, about 20 cc to about 50 cc, about 20 cc to about 60 cc, about 20 cc to about 70 cc, about 20 cc to about 80 cc, about 30 cc to about 40 cc, about 30 cc to about 50 cc, about 30 cc to about 60 cc, about 30 cc to about 70 cc, about 30 cc to about 80 cc, about 40 cc to about 50 cc, about 40 cc to about 60 cc, about 40 cc to about 70 cc, about 40 cc to about 80 cc, about 50 cc to about 60 cc, about 50 cc to about 70 cc, about 50 cc to about 80 cc, about 60 cc to about 70 cc, about 60 cc to about 80 cc, or from about 70 cc to about 80 cc. The aspiration syringe can be less than about 10 cc, less than about 15 cc, less than about 20 cc, less than about 25 cc, less than about 30 cc, less than about 35 cc, less than about 40 cc, less than about 45 cc, less than about 50 cc, less than about 55 cc, less than about 60 cc, less than about 65 cc, less than about 70 cc, less than about 75 cc, or less than about 80 cc. The aspiration syringe can be greater than about 10 cc, greater than about 15 cc, greater than about 20 cc, greater than about 25 cc, greater than about 30 cc, greater than about 35 cc, greater than about 40 cc, greater than about 45 cc, greater than about 50 cc, greater than about 55 cc, greater than about 60 cc, greater than about 65 cc, greater than about 70 cc, greater than about 75 cc, or greater than about 80 cc. The device/wire 2200 can be manually pulled into the central lumen of the guide catheter and upon entering the constrained central lumen of the guide catheter the device/wire deforms and straightened to be withdrawn out of the body.

EPDs can be subjected to inadvertent pull/push traction forces by the user during their deployment, catheter exchange and interventional procedure. This can lead to filter malposition or dislodgement from its ideal in-flush apposition state with the vessel wall, which may result in particulate dislodgment from the filter cone 2207 and distal embolization or dissection of the vessel wall. Hence, in some embodiments, a short helical loop is configured and memorized over the straight portion of the device/wire near the tip of the cone segment 2207 during the heat treatment process. This safety feature can take the impact of inadvertent traction forces from the straight segment of the wire and thus may buffer the filter-cone segment 2207 from disfigurement due to its disposition in between the straight and cone segments 2207 of the wire/device 2200.

In some cases, the filter-cone 2207 coils are coated with a polymer with positive electrostatic charges to boost its emboli capturing efficacy. Cholesterol and phospholipid particles inside atherosclerotic plaque and/or occlusion masses may be negatively charged due to abundance of singlet oxygen molecules (O—) generated from chronic inflammation. Atheroemboli generated during interventions may be attracted and adhered to these oppositely charged polymer molecules.

In some cases, the surface of filter cone 2207 coils is coated with PEGylated antibody to high-density lipoproteins (HDLs). The PEG-protein conjugates can have several advantages: a prolonged residence in body, a decreased degradation by metabolic enzymes, and a reduction or elimination of protein immunogenicity. Atherosclerotic plaque and/or occlusion masses are rich in macrophages whose surfaces may be abundantly coated with HDL receptors. In this way, both micro and macro atheroemboli generated during interventions can be effectively captured downstream by antigen/antibody interactions over the surface of filter cone 2207 coils.

The embolic protection device can also be used during lower or upper limb deep venous thrombectomy interventions as a temporary IVC or SVC filter (Inferior Vena Cava or Superior Vena Cava) to minimize embolic material traversal into the pulmonary circulation.

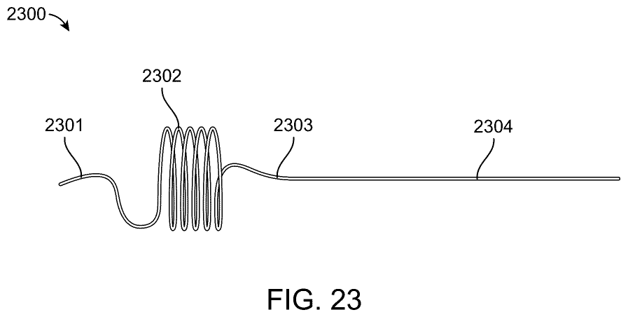

FIG. 23 shows a side view of an example embodiment of a coiled tip guidewire 2300 for use in devices described throughout the application. Guidewire 2300 can comprise flexible segment 2301, coiled segment 2302, transitional segment 2303, and stiff segment 2304. Coiled tip guidewire 2300 can be inserted into a guidewire lumen of a device. The device can be any of the devices described throughout the application, as this is an accessory that can function with any of the devices. The diameter of the guidewire can be non-uniform. The diameter can be uniform. The diameter can be approximately 0.014″ or 0.018″. In some cases, the diameter is approximately 0.014″ for coronary applications. Both floppy segment 2301 and coiled segment 2302 can be made of a flexible material. However, coiled segment 2302 can be pre-shaped and comprises of a memory shape material such as NiTi. Coiled segment 2302 can be set to coil when advanced from the guidewire lumen of a device. Transitional segment 2303 can have a transitional stiffness that is stiffer than segments 2301 and 2302 but lesser stiff than segment 2304. The coil in its deployed configured can be just distal to the device central lumen. Then, the wire segment of intermediate stiffness can be oriented across the bends to give better rail support to bulky interventional devices, such as coronary occlusion crossing devices.

Guidewire 2300 can be beneficial when the proximal end of occlusion is located just distal to a branch point or a bend and a device may have difficulty being tracked over the bends over a flexible distal segment of a guidewire. Instead, guidewire 2300 can be advanced through a device guidewire lumen until the coiled segment 2302 is disposed just proximal to the proximal end of the occlusion. At this point the transitional segment 2303 may be located across the acute vessel bends or at branch points. The device shaft can track over the transitional segment with intermedial stiffness 2303 and thus navigate easily past the bend or branch point.

In some cases, the total length of the flexible segments of a regular guidewire can be from about 30 mm to about 50 mm. The total length of the flexible segments can be less than about 20 mm, less than about 30 mm, less than about 40 mm, less than about 50 mm, or less than about 60 mm. The total length of the flexible segments can be greater than about 20 mm, greater than about 30 mm, greater than about 40 mm, greater than about 50 mm, or greater than about 60 mm. The coiled segment 2302 can be used to condense the from about 30 mm to about 50 mm of flexible length into less than or equal to about 1 cm. This condensed coil 2302 can be located between the bend angle and the proximal part of the occlusion. In some embodiments, the total length, counted as the space between coils and the coils themselves, of the flexible segments of guidewire 2300 is approximately 15 mm.

Occlusions may be formed with their proximal sides close to bends in arteries or branch points. In some cases, the distance between the proximal side of the occlusion and the bend or branch point may be from about 0.5 cm to about 1 cm. In some cases, the distance between the proximal side of the occlusion and the bend or branch point may be less than about 0.5 cm, less than about 1 cm, less than about 1.5 cm, or less than about 2 cm. In some cases, the distance between the proximal side of the occlusion and the bend or brand point may be greater than about 0.5 cm, greater than about 1 cm, greater than about 1.5 cm, or greater than about 2 cm. In some cases, an uncoiled flexible end of a guidewire may not be able to be stable in such anatomy and may not be able to support the device shaft during crossing over these bends. By condensing the flexible end into a coil formation, the coil can remain in the limited distance between the proximal side of the occlusion and the bend or branch point while retaining stability.

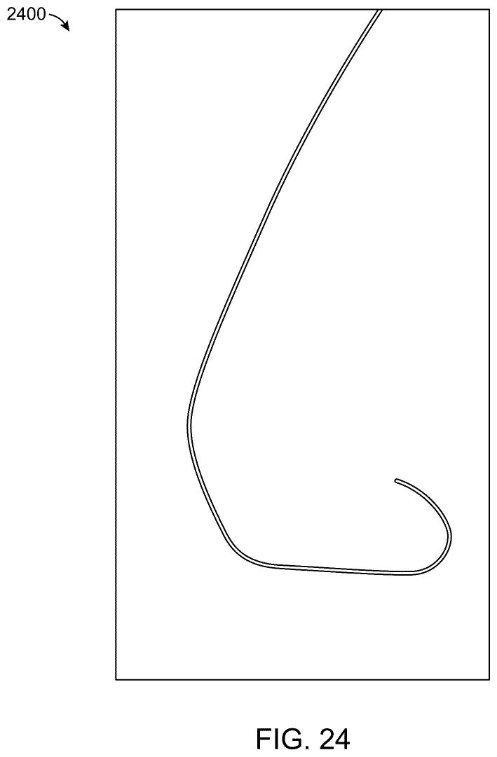

FIG. 24 shows a top view of an embodiment of a steering stylet 2400 for deflection of tips of example devices described throughout the application. Steering stylet 2400 can comprise a nitinol-based wire. In some cases, the stylet 2400 is pre-shaped and memory-set. In some cases, the stylet 2400 is pre-shaped and memory-set into a shape similar to that of FIG. 24. The stylet can be inserted into the guidewire lumen of a device. The device can be any of the devices described throughout the application, as this is an accessory that can function with any of the devices. When introduced into the guidewire lumen of the device, the stylet may get straightened out and stay relatively straight until it is advanced close to the device's distal end. In some embodiments, the distal 5 cm to 20 cm of any of the devices described throughout the application may be relatively more flexible than the rest of the device shaft. Accordingly, once the stylet reaches this point, it may become mechanically dominant in stiffness over the device shaft. Hence, at this point the stylet can start bending inside the lumen of the device due to its inherent shape memory effect. As the stylet is relatively stiffer compared to the distal end of the device, the stylet, when bent, can deflect the device tip. The angle of this device tip deflection and point of inflection on the device distal end from its tip varies and can depend on several factors. For example, the factors can include the diameter of the lumen of the blood vessel in which the device is located, the kind of shape given to the stylet's distal end at manufacturing, the flexibility of the device distal end itself, etc.

FIGS. 25A-25D show side views (25A-25C) and a perspective inner view (25D) of a hybrid guide catheter 2500 (also called a “hybrid guide and guide extensions catheter”) for optionally facilitating use of devices described throughout the application. FIG. 25A shows an external side view of hybrid catheter 2500 comprising proximal section 2502, distal section 2510, and actuation system 2520. FIG. 25B shows a magnified external side view of the proximal section 2502 of the hybrid catheter 2500 comprising outer shaft 2508, and actuation system 2520. FIG. 25C shows a magnified external side view of distal section 2510 of the hybrid catheter 2500 comprising inner shaft 2506 and outer shaft 2508. FIG. 25D shows a magnified internal perspective view of actuation system 2520 comprising inner shaft 2506, outer shaft 2508, actuation knob 2524, and actuation slider 2522. This hybrid guide catheter 2500 can be used for coronary procedures. Hybrid catheter 2500 can function as an accessory to the primary devices described above. In addition to the devices described above that can be used for coronary procedures, hybrid catheter 2500 may be compatible with coronary guidewires and other interventional devices used in coronary interventions, such as coronary crossing devices.

The hybrid catheter 2500 can comprise a seamless continuation of the lumen of inner shaft 2506 and outer shaft 2508. Guidewires and/or coronary interventional device can be inserted into the lumen of hybrid catheter 2500. The lumen can be used to introduce guidewires and/or coronary interventional device into the blood vessel and help guide the wire and device close to the occluded site. Actuation slide 2522 can help to push inner shaft 2506. The actuation knob 2524 can act as a housing for the actuation slider. In some cases, the actuation knob 2524 is mobile and can be rotated to rotate the inner shaft as needed to navigate small coronary arteries. The outer shaft can be strengthened with wire braiding. It may have a thermal shape set similar to a coronary guidecatheter, which can provide stability and column support for the interventional devices. The inner shaft may be thinner and more flexible than the outer shaft to facilitate smooth tracking inside the tortuous coronary anatomy. This hybrid catheter 2500 can add simplicity, stability, safety, and effectiveness to coronary interventional procedure. In the normal state, the tip of the inner shaft can be inside the outer shaft. During its usage, the hybrid guidecatheter can be introduced over a guidewire into the aorta and the outer shaft tip may be engaged into the coronary ostium. The inner shaft 2506 can be advanced distally deeper into the coronary artery, by distally sliding the actuator located at the proximal end. In some cases, the inner shaft 2506 can be advanced from about 5 cm to about 10 cm.

In some cases, the inner shaft 2506 can be advanced less than about 5 cm. In some cases, the inner shaft 2506 can be advanced greater than about 10 cm. In some cases, the inner shaft 2506 can be by itself or coaxially over a coronary guidewire or over a small coronary angioplasty balloon using an “inchworm” technique. The small balloon may be from about 2 mm to about 3 mm in diameter. The small balloon may be less than about 2 mm in diameter. The small balloon may be greater than about 3 mm in diameter. The “inchworm” technique can include positioning an inflated balloon halfway at the distal tip of a catheter during delivery and advancing a guide catheter immediately as the balloon is deflated, thus advancing the guide catheter distally over the deflated balloon. Once the inner shaft is positioned deeper inside the coronary artery, any coronary guidewire, crossing devices or other interventional catheters can be used through the lumen of inner shaft for interventions. The presence of the inner shaft across the coronary ostium and extending deeper inside the coronary artery can provide support and stability during the intervention and also reduces the potential loss of effective tip load of coronary crossing devices and guidewires.

### Methods

### Methods for Crossing Occlusions

Provided herein are methods for crossing occlusions. Any of the devices described herein comprising any of the features described herein can be used in any of the methods described below. In some cases, the methods can comprise drilling, cutting, penetrating, pulverizing, blunt dissection or any combination thereof. In some cases, a pushing force, rotation, or both can be used to cross the occlusion. In some cases, a user can simultaneously exert a pushing force and rotate the device. In some cases, simultaneously pushing and rotating can be more effective at crossing occlusions than using one or the other method alone, wherein effectiveness is measured by length of time of the crossing. In some cases, once one or more devices cross the occlusion, the one or more devices can be removed and leave a tunnel in the occlusion. In some embodiments, the tunnel is formed by a dottering effect, wherein the plaque and/or occlusion mass comprising the occlusion can be compressed and radially displaced to the sides of the vessel wall. This way, a tunnel can be formed without cutting away and removing the plaque and/or occlusion mass as in an atherectomy. In some embodiments, the vessel wall is not stretched, extended, or expanded. In some cases, the vessel wall is not dissected, cut, perforated, or damaged.

FIGS. 26A-26C and FIGS. 26D-26K show a device as described herein for crossing occlusions advanced through a chronic total occlusion CTO in a blood vessel (BV). The distal end portion 12 of the device can be advanced so that the cutting loop 20 lies immediately proximal to a proximal surface of the chronic total occlusion CTO. In some cases, a guidewire can be extended to the proximal surface of the CTO prior to the device so the device can follow over the wire. The user can manually rotate the cutting tip 18 and cutting loop 20 while advancing the device to penetrate through the proximal face of the occlusion and begin to create a central passage therethrough.

The cutting loop 20 can be used to pulverize the occlusion, then the conical tip and centering cage can separate the pulverized plaque and/or occlusion mass and compress it along the sides of the vessel wall by moving past the sides of the wall. The spiral centering springs of centering cage 22 can engage the inner walls of the blood vessel and maintain centering of the distal region 12 of the device as the device is advanced. The flat surfaces of the springs can atraumatically engage the vessel wall while the low width of each spring allows the centering cage to pass through the occlusive material with reduced resistance. While passing through, the centering cage exerts a minimal pressure on the surrounding plaque and/or occlusion mass and, through the plaque and/or occlusion mass, on the walls of the vessel, of less than 2 mm Hg. The centering cage can exert a pressure of less than 1 mm Hg. The centering cage can exert a pressure of from about 1 mm Hg to about 2 mm Hg. The centering cage can exert a pressure of greater than 2 mm Hg. In some cases, the pressure exerted may be from about 2 mm Hg to about 10 mm Hg. In some cases, the pressure exerted may be from about 2 mm Hg to about 4 mm Hg, about 2 mm Hg to about 6 mm Hg, about 2 mm Hg to about 8 mm Hg, about 2 mm Hg to about 10 mm Hg, about 4 mm Hg to about 6 mm Hg, about 4 mm Hg to about 8 mm Hg, about 4 mm Hg to about 10 mm Hg, about 6 mm Hg to about 8 mm Hg, about 6 mm Hg to about 10 mm Hg, or from about 8 mm Hg to about 10 mm Hg. In some cases, there is minimal or no stretching of the vessel walls. In some cases, due to this low pressure, even though the device compresses the pulverized plaque and/or occlusion mass against the sides of the vessel, it does not cause expansion of the vessel wall. Accordingly, when passing through the blood vessel on its way to an occlusion mass, the devices described herein likewise do not cause expansion, widening, extension, stretching, or any combination thereof. Barotrauma, or pressure-based injury, to vessel walls can occur due to a high strain rate. As such, stretching the vessel wall slowly may not cause as much damage, if any. The devices described herein may have a strain rate of zero or one that is negligible to vessel walls in non-balloon variations due to lack of lateral force application and thus no active application of lateral strain rate. As such, there may be no vessel dilation or injury. The device can be incrementally advanced as the user manually rotates the cutting tip and observes the progress fluoroscopically. Although the reference numbers are those that refer to device 10, these methods can be accomplished by any of the devices disclosed herein.

In some cases, the centering cage 22 centers the cutting loop such that rotating the cutting loop results in a circle, resulting in a circular tunnel as shown in FIG. 1A. In some cases, the centering cage 22 gives the cutting loop some degree of freedom such that rotating the cutting loop can result in a circle, ellipse, oval, or any other elliptical shape. The degrees of freedom can result in a wider diameter tunnel than the diameter of the centering cage 22. In devices that don't have a centering cage, the tunnel shape can similarly result in a circle, ellipse, oval, or any other elliptical shape with a wider diameter tunnel than the diameter of the cutting loop or tip.

In some cases, the degrees of freedom result in a channel that is from about 10% to about 20% larger than the largest profile of a device as described herein. In some cases, the degrees of freedom result in a channel that is from about 10% to about 12%, about 10% to about 14%, about 10% to about 16%, about 10% to about 18%, about 10% to about 20%, about 12% to about 14%, about 12% to about 16%, about 12% to about 18%, about 12% to about 20%, about 14% to about 16%, about 14% to about 18%, about 14% to about 20%, about 16% to about 18%, about 16% to about 20%, or from about 18% to about 20%. In some cases, the degrees of freedom result in a channel that is than about 10%, less than about 12%, less than about 14%, less than about 16%, less than about 18%, or less than about 20% larger than the largest profile of a device as described herein. In some cases, the degrees of freedom result in a channel that is than about 10%, greater than about 12%, greater than about 14%, greater than about 16%, greater than about 18%, or greater than about 20% larger than the largest profile of a device as described herein.

Due to the degrees of freedom, the diameter and area of the channel can vary. In some cases, where there was no prior channel, the devices and methods described herein can create a channel of the sizes described below. In some cases, where there was a prior channel, the devices and methods described herein can enlarge the existing channel by the amounts described below.

In some cases, the diameter or addition to the diameter of the channel can be from about 0.5 mm to about 3.7 mm. In some cases, the diameter or addition to the diameter of the channel can be from about 0.5 mm to about 0.9 mm, about 0.5 mm to about 1.3 mm, about 0.5 mm to about 1.7 mm, about 0.5 mm to about 2.1 mm, about 0.5 mm to about 2.5 mm, about 0.5 mm to about 2.9 mm, about 0.5 mm to about 3.3 mm, about 0.5 mm to about 3.7 mm, about 0.9 mm to about 1.3 mm, about 0.9 mm to about 1.7 mm, about 0.9 mm to about 2.1 mm, about 0.9 mm to about 2.5 mm, about 0.9 mm to about 2.9 mm, about 0.9 mm to about 3.3 mm, about 0.9 mm to about 3.7 mm, about 1.3 mm to about 1.7 mm, about 1.3 mm to about 2.1 mm, about 1.3 mm to about 2.5 mm, about 1.3 mm to about 2.9 mm, about 1.3 mm to about 3.3 mm, about 1.3 mm to about 3.7 mm, about 1.7 mm to about 2.1 mm, about 1.7 mm to about 2.5 mm, about 1.7 mm to about 2.9 mm, about 1.7 mm to about 3.3 mm, about 1.7 mm to about 3.7 mm, about 2.1 mm to about 2.5 mm, about 2.1 mm to about 2.9 mm, about 2.1 mm to about 3.3 mm, about 2.1 mm to about 3.7 mm, about 2.5 mm to about 2.9 mm, about 2.5 mm to about 3.3 mm, about 2.5 mm to about 3.7 mm, about 2.9 mm to about 3.3 mm, about 2.9 mm to about 3.7 mm, and finally, about 3.3 mm to about 3.7 mm. In some cases, the diameter or addition to the diameter of the channel can be less than about 0.5 mm, less than about 0.9 mm, less than about 1.3 mm, less than about 1.7 mm, less than about 2.1 mm, less than about 2.5 mm, less than about 2.9 mm, less than about 3.3 mm, or less than about 3.7 mm. In some cases, the diameter or addition to the diameter of the channel can be greater than about 0.5 mm, greater than about 0.9 mm, greater than about 1.3 mm, greater than about 1.7 mm, greater than about 2.1 mm, greater than about 2.5 mm, greater than about 2.9 mm, greater than about 3.3 mm, or greater than about 3.7 mm.

In some cases, the diameter or addition to the diameter of the channel can be from about 0.5 mm to about 5 mm. In some cases, the diameter or addition to the diameter of the channel can be from about 0.5 mm to 1 mm, about 0.5 mm to 1.5 mm, about 0.5 mm to 2 mm, about 0.5 mm to 2.5 mm, about 0.5 mm to 3 mm, about 0.5 mm to 3.5 mm, about 0.5 mm to 4 mm, about 0.5 mm to 4.5 mm, about 0.5 mm to 5 mm, about 1 mm to 1.5 mm, about 1 mm to 2 mm, about 1 mm to 2.5 mm, about 1 mm to 3 mm, about 1 mm to 3.5 mm, about 1 mm to 4 mm, about 1 mm to 4.5 mm, about 1 mm to 5 mm, about 1.5 mm to 2 mm, about 1.5 mm to 2.5 mm, about 1.5 mm to 3 mm, about 1.5 mm to 3.5 mm, about 1.5 mm to 4 mm, about 1.5 mm to 4.5 mm, about 1.5 mm to 5 mm, about 2 mm to 2.5 mm, about 2 mm to 3 mm, about 2 mm to 3.5 mm, about 2 mm to 4 mm, about 2 mm to 4.5 mm, about 2 mm to 5 mm, about 2.5 mm to 3 mm, about 2.5 mm to 3.5 mm, about 2.5 mm to 4 mm, about 2.5 mm to 4.5 mm, about 2.5 mm to 5 mm, about 3 mm to 3.5 mm, about 3 mm to 4 mm, about 3 mm to 4.5 mm, about 3 mm to 5 mm, about 3.5 mm to 4 mm, about 3.5 mm to 4.5 mm, about 3.5 mm to 5 mm, about 4 mm to 4.5 mm, about 4 mm to 5 mm, or from about 4.5 mm to 5 mm. In some cases, the diameter or addition to the diameter of the channel can be less than about 0.5 mm, less than about 1 mm, less than about 1.5 mm, less than about 2 mm, less than about 2.5 mm, less than about 3 mm, less than about 3.5 mm, less than about 4 mm, less than about 4.5 mm, or less than about 5 mm. In some cases, the diameter or addition to the diameter of the channel can be greater than about 0.5 mm, greater than about 1 mm, greater than about 1.5 mm, greater than about 2 mm, greater than about 2.5 mm, greater than about 3 mm, greater than about 3.5 mm, greater than about 4 mm, greater than about 4.5 mm, or greater than about 5 mm.

In some cases, the cross-sectional area or addition to the cross-sectional area of the channel can be from about 0.5 mm² and about 13.5 mm². In some cases, the cross-sectional area or addition to the cross-sectional area of the channel can be from about 0.5 mm² and about 18.5 mm². In some cases, the area or addition to the area of the channel can be from about 0.5 mm² to about 2.5 mm², about 0.5 mm² to about 4.5 mm², about 0.5 mm² to about 6.5 mm², about 0.5 mm² to about 8.5 mm², about 0.5 mm² to about 10.5 mm², about 0.5 mm² to about 12.5 mm², about 0.5 mm² to about 14.5 mm², about 0.5 mm² to about 16.5 mm², about 0.5 mm² to about 18.5 mm², about 2.5 mm² to about 4.5 mm², about 2.5 mm² to about 6.5 mm², about 2.5 mm² to about 8.5 mm², about 2.5 mm² to about 10.5 mm², about 2.5 mm² to about 12.5 mm², about 2.5 mm² to about 14.5 mm², about 2.5 mm² to about 16.5 mm², about 2.5 mm² to about 18.5 mm², about 4.5 mm² to about 6.5 mm², about 4.5 mm² to about 8.5 mm², about 4.5 mm² to about 10.5 mm², about 4.5 mm² to about 12.5 mm², about 4.5 mm² to about 14.5 mm², about 4.5 mm² to about 16.5 mm², about 4.5 mm² to about 18.5 mm², about 6.5 mm² to about 8.5 mm², about 6.5 mm² to about 10.5 mm², about 6.5 mm² to about 12.5 mm² about 6.5 mm² to about 14.5 mm², about 6.5 mm² to about 16.5 mm², about 6.5 mm² to about 18.5 mm², about 8.5 mm² to about 10.5 mm², about 8.5 mm² to about 12.5 mm², about 8.5 mm² to about 14.5 mm², about 8.5 mm² to about 16.5 mm², about 8.5 mm² to about 18.5 mm², about 10.5 mm² to about 12.5 mm², about 10.5 mm² to about 14.5 mm², about 10.5 mm² to about 16.5 mm², about 10.5 mm² to about 18.5 mm², about 12.5 mm² to about 14.5 mm², about 12.5 mm² to about 16.5 mm² about 12.5 mm² to about 18.5 mm², about 14.5 mm² to about 16.5 mm², about 14.5 mm² to about 18.5 mm², and finally, about 16.5 mm² to about 18.5 mm². In some cases, the area or addition to the area of the channel can be less than about 0.5 mm², less than about 1.5 mm², less than about 2.5 mm², less than about 3.5 mm², less than about 4.5 mm², less than about 5.5 mm², less than about 6.5 mm², less than about 7.5 mm², less than about 8.5 mm², less than about 9.5 mm², less than about 10.5 mm², less than about 11.5 mm², less than about 12.5 mm², less than about 13.5 mm², less than about 14.5 mm², less than about 15.5 mm², less than about 16.5 mm², less than about 17.5 mm², or less than about 18.5 mm². In some cases, the area or addition to the area of the channel can be greater than about 0.5 mm², greater than about 1.5 mm², greater than about 2.5 mm², greater than about 3.5 mm², greater than about 4.5 mm², greater than about 5.5 mm², greater than about 6.5 mm² greater than about 7.5 mm², greater than about 8.5 mm², greater than about 9.5 mm², greater than about 10.5 mm², greater than about 11.5 mm², greater than about 12.5 mm², greater than about 13.5 mm², greater than about 14.5 mm², greater than about 15.5 mm², greater than about 16.5 mm², greater than about 17.5 mm², or greater than about 18.5 mm².

In some cases, the cross-sectional area or addition to the cross-sectional area of the channel can be from about 2 mm² and about 18 mm². In some cases, the cross-sectional area or addition to the cross-sectional area of the channel can be from about 2 mm² to 4 mm², about 2 mm² to 6 mm², about 2 mm² to 8 mm², about 2 mm² to 10 mm², about 2 mm² to 12 mm², about 2 mm² to 14 mm² about 2 mm² to 16 mm², about 2 mm² to 18 mm², about 4 mm² to 6 mm², about 4 mm² to 8 mm² about 4 mm² to 10 mm² about 4 mm² to 12 mm², about 4 mm² to 14 mm², about 4 mm² to 16 mm², about 4 mm² to 18 mm², about 6 mm² to 8 mm², about 6 mm² to 10 mm², about 6 mm² to 12 mm² about 6 mm² to 14 mm², about 6 mm² to 16 mm², about 6 mm² to 18 mm², about 8 mm² to 10 mm² about 8 mm² to 12 mm², about 8 mm² to 14 mm², about 8 mm² to 16 mm², about 8 mm² to 18 mm², about 10 mm² to 12 mm², about 10 mm² to 14 mm², about 10 mm² to 16 mm², about 10 mm² to 18 mm², about 12 mm² to 14 mm², about 12 mm² to 16 mm², about 12 mm² to 18 mm², about 14 mm² to 16 mm², about 14 mm² to 18 mm², about 16 mm² to 18 mm². In some cases, the cross-sectional area or addition to the cross-sectional area of the channel can be less than about 2 mm², less than about 3 mm², less than about 4 mm², less than about 5 mm², less than about 6 mm², less than about 7 mm², less than about 8 mm², less than about 9 mm², less than about 10 mm², less than about 11 mm² less than about 12 mm², less than about 13 mm², less than about 14 mm², less than about 15 mm², less than about 16 mm², less than about 17 mm², less than about 18 mm². In some cases, the cross-sectional area or addition to the cross-sectional area of the channel can be greater than about 2 mm², greater than about 3 mm², greater than about 4 mm², greater than about 5 mm², greater than about 6 mm², greater than about 7 mm², greater than about 8 mm², greater than about 9 mm², greater than about 10 mm², greater than about 11 mm², greater than about 12 mm², greater than about 13 mm², greater than about 14 mm², greater than about 15 mm², greater than about 16 mm², greater than about 17 mm², greater than about 18 mm².

In some cases, channel diameter can be enlarged by about 1 mm to about 4 mm in a vessel size of from about 1.5 mm to about 15 mm.

In some embodiments, enlargement of the channel via the devices and methods described herein can provide a percentage improvement in channel diameter from about 5% to about 80% (e.g., 105% and 180% of the original channel size). The percentage improvement in channel diameter can be from about 5% to about 20%, about 5% to about 35%, about 5% to about 50%, about 5% to about 65%, about 5% to about 80%, about 20% to about 35%, about 20% to about 50%, about 20% to about 65%, about 20% to about 80%, about 35% to about 50%, about 35% to about 65%, about 35% to about 80%, about 50% to about 65%, about 50% to about 80%, or from about 65% to about 80%. The percentage improvement in channel diameter can be less than about 5%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, or less than about 80%. The percentage improvement in channel diameter can be greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, or greater than about 80%.

The procedure may be complete when the distal tip of the device passes through a distal face of the occlusion, as shown in FIG. 26C. After the device has reached the position shown in FIG. 26C, a guidewire GW may be placed, the device can be removed and/or other interventional procedures may be performed.

In some cases, one or more devices described herein can be used to tunnel through an occlusion. A guidewire, catheter, or other device can then be extended past the occlusion, as in FIG. 26C. In some cases, one or more devices described herein can be used to cross partway through an occlusion, with the remainder of the occlusion being traversed by a guidewire. In some embodiments, one device as described herein can cross partway through the occlusion and can then be switched out for another device as described herein to cross the remainder of the occlusion. This can be beneficial when a single occlusion has multiple properties, for example part of the occlusion is soft and part is hard.

Once withdrawn after crossing the occlusion, the device may leave a tunnel or a channel. A subsequent device comprising a medicine coating the distal end can be inserted to apply the medicine to the walls of the tunnel. In some cases, for example when the device comprises a balloon, the balloon can have medicine coating its walls for application after crossing the occlusion. This way, only one device can be used to cross the occlusion, leave behind a tunnel, and administer medication to the tunnel and/or blood vessel.

This method can be used effectively on soft and moderately hard plaque and/or occlusion masses that constitute approximately 85% of occlusions. Unlike guidewires alone, which are only able to impart a puncture strength of about 20 g to about 40 g. Moderately hard plaque and/or occlusion masses can require up to about 300 g of strength to puncture. The devices described herein can impart up to approximately 300 g of strength, so they are able to puncture soft and moderately hard plaque and/or occlusion masses.

FIGS. 26D-26G show a device as described herein for crossing occlusions using Finite Element Analysis. The figures can comprise snapshots of the entry of a device into an occlusion based on a ‘disappearing elements’ model. As such, the actual concentration of forces at various touch points between the device features and the surrounding occlusive mass are higher as the mass may be compressed in that area. As such, there may be more stress concentration on those spots than shown, but not reaching all the way to the outer perimeter of the blue zone. The vessel wall may be kept unperturbed for negligible, if any, lateral barotrauma to occur to the blood vessel wall.

FIGS. 2611-26K likewise show a device as described herein for crossing occlusions. The figures can comprise snapshots of the entry of a device into an occlusion. The vessel wall may be kept unperturbed for negligible, if any, lateral barotrauma to occur to the blood vessel wall.

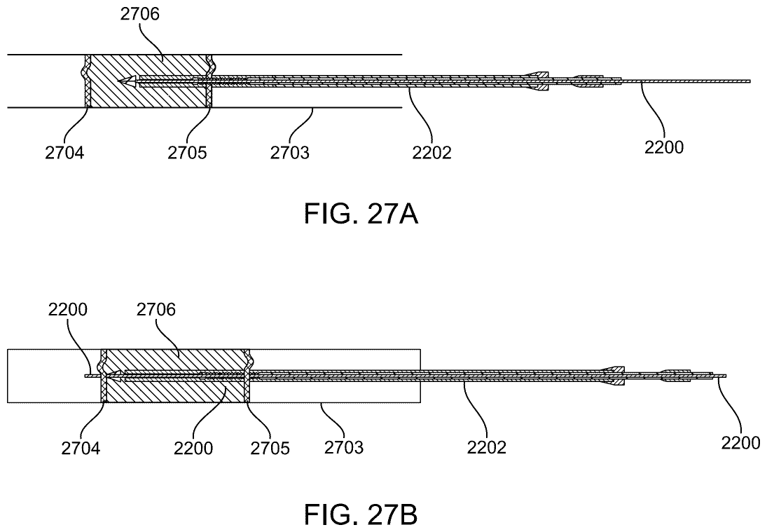

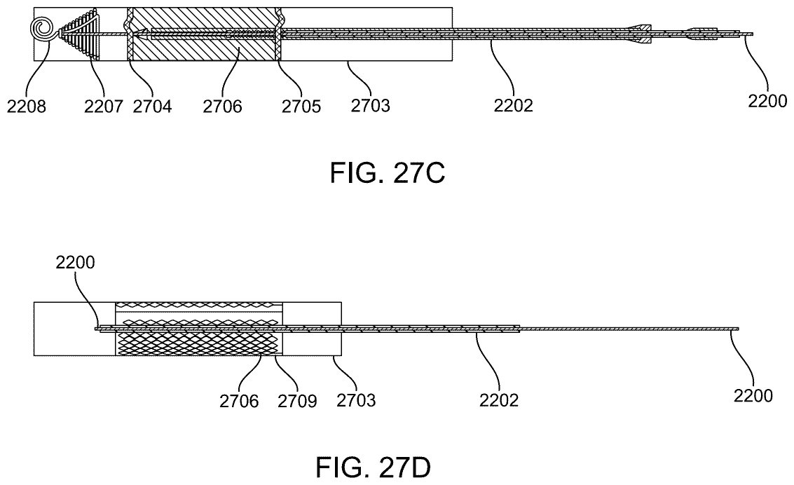

FIGS. 27A-27D show the use of an embolic protection device 2200 as described herein for capturing debris released from crossed occlusions in accordance with embodiments described herein.

FIG. 27A shows embolic protection device 2200 partially crossing an occlusion comprising embolic protection device 2200, delivery catheter or interventional device 2202, proximal surface of the occlusion 2705, distal surface of the occlusion 2704, blood vessel 2703, and plaque and/or occlusion mass 2706. FIG. 27B shows embolic protection device 2200 fully crossing an occlusion comprising embolic protection device 2200, delivery catheter or interventional device 2202, proximal surface of the occlusion 2705, distal surface of the occlusion 2704, blood vessel 2703, and plaque and/or occlusion mass 2706. FIG. 27C shows embolic protection device 2200 in use comprising embolic protection device 2200, delivery catheter or interventional device 2202, proximal surface of the occlusion 2705, distal surface of the occlusion 2704, blood vessel 2703, plaque and/or occlusion mass 2706, filter cone 2207, and pig-tail tip 2208. FIG. 27D shows embolic protection device 2200 fully crossing an occlusion comprising embolic protection device 2200, delivery catheter or interventional device 2202, blood vessel 2703, plaque and/or occlusion mass 2706, and stent 2709.

In some embodiments, embolic protection device 2200 comprises a guidewire inside of a delivery or interventional catheter 2202. In some cases, the delivery or interventional catheter 2202 can cross an occluded area of a blood vessel using the methods described herein. During crossing, plaque and/or occlusion mass and other debris can be dislodged from the occluded area. Once the delivery or interventional catheter 2202 has crossed the occluded area, a user can extend embolic protection device/guidewire 2200 past the end of the delivery or interventional catheter 2202. Once outside of the confining lumen, EPD 2200 returns to its memorized shape comprising a coiled filter cone 2207 and a pig-tail tip 2208. The spaces between the coils of the filter cone 2207 can be less than 50 microns, the filter cone can capture debris that is larger than 50 microns. As red blood cells are approximately 10 microns in size, they are not blocked by the filter cone 2207. As described above, small debris less than 50 microns in size are not dangerous for blood vessels. As such, the filter cone 2207 can capture dangerous debris without impeding the flow of blood.

At the completion of intervention/crossing and once the filter cone 2207 and pig-tail tip 2208 have been allowed to form, the delivery or interventional catheter can be withdrawn from the blood vessel over the device/wire 2200. A user can then insert from about a 6 F to about a 7 F multipurpose or similarly shaped guide catheter over the device/wire 2200. The tip of this guide catheter can be carefully advanced close to the tip of the filter cone 2207. In some cases, it can be advanced under fluoroscopic guidance. An aspiration syringe from about 30 cc to about 60 cc can be attached to the proximal end of the guide catheter and the collected debris from the cone filter 2207 can be aspirated with or without saline flush.

The aspiration syringe can be from about 10 cc to about 80 cc. The aspiration syringe can be from about 10 cc to about 20 cc, about 10 cc to about 30 cc, about 10 cc to about 40 cc, about 10 cc to about 50 cc, about 10 cc to about 60 cc, about 10 cc to about 70 cc, about 10 cc to about 80 cc, about 20 cc to about 30 cc, about 20 cc to about 40 cc, about 20 cc to about 50 cc, about 20 cc to about 60 cc, about 20 cc to about 70 cc, about 20 cc to about 80 cc, about 30 cc to about 40 cc, about 30 cc to about 50 cc, about 30 cc to about 60 cc, about 30 cc to about 70 cc, about 30 cc to about 80 cc, about 40 cc to about 50 cc, about 40 cc to about 60 cc, about 40 cc to about 70 cc, about 40 cc to about 80 cc, about 50 cc to about 60 cc, about 50 cc to about 70 cc, about 50 cc to about 80 cc, about 60 cc to about 70 cc, about 60 cc to about 80 cc, or from about 70 cc to about 80 cc. The aspiration syringe can be less than about 10 cc, less than about 15 cc, less than about 20 cc, less than about 25 cc, less than about 30 cc, less than about 35 cc, less than about 40 cc, less than about 45 cc, less than about 50 cc, less than about 55 cc, less than about 60 cc, less than about 65 cc, less than about 70 cc, less than about 75 cc, or less than about 80 cc. The aspiration syringe can be greater than about 10 cc, greater than about 15 cc, greater than about 20 cc, greater than about 25 cc, greater than about 30 cc, greater than about 35 cc, greater than about 40 cc, greater than about 45 cc, greater than about 50 cc, greater than about 55 cc, greater than about 60 cc, greater than about 65 cc, greater than about 70 cc, greater than about 75 cc, or greater than about 80 cc.

The device/wire 2200 can be manually pulled into the central lumen of the guide catheter and upon entering the constrained central lumen of the guide catheter the device/wire deforms and straightened to be withdrawn out of the body.

### Methods for Inserting a Device for Crossing Occlusions

Described herein are methods for inserting the devices described herein to reach occlusions. In some cases, inserting can comprise insertion at a radial, pedal, brachial, femoral, or popliteal artery or vein, or any combination thereof.

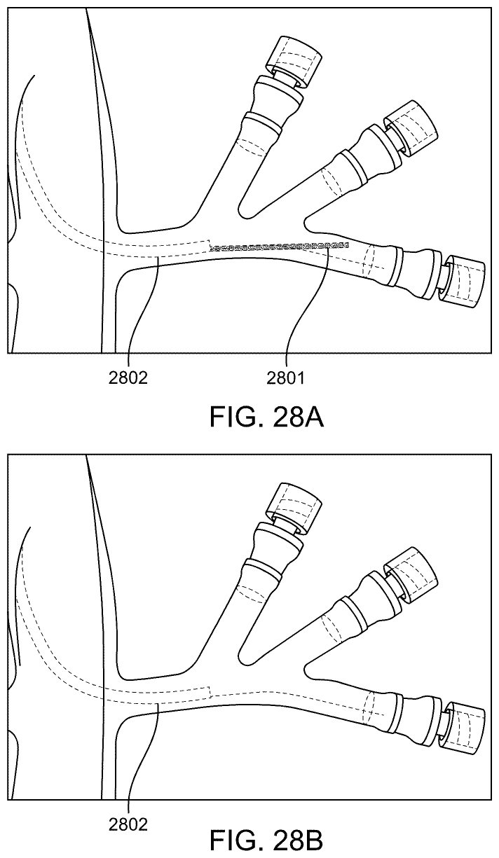

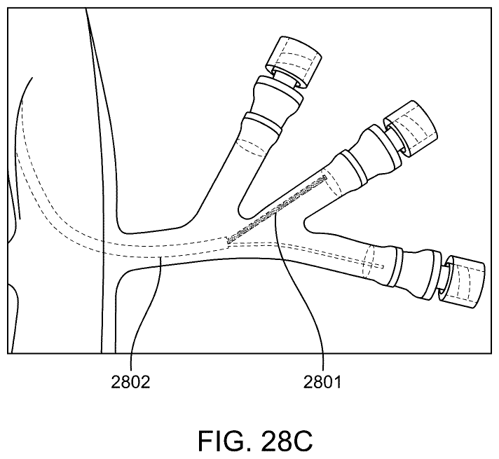

In some cases, inserting the devices described herein to reach occlusions can comprise steering through branching and/or tortuous vessels. FIGS. 28A-28C show the use of a steering stylet in deflecting the tip and guidewire 2801 away from the direction of the shaft 2802 and away from an original vessel to a desired vessel at a branching point in accordance with methods described herein. FIG. 28A shows a side view of the tip and guidewire 2801 proceeding from shaft 2802 in a non-desired direction. FIG. 28B shows a side view of the guidewire 2801 being pulled back into the shaft 2802 and guidewire lumen. FIG. 28C shows a side view of tip and guidewire 2801 proceeding from shaft 2802 in a desired direction after use of the stylet. As described above, the stylet can be pre-shaped and memory-set. Once the stylet reaches the distal end of a device as described throughout the application, it may become stiffer than the stiffness of the last approximately 20 cm segment of device shaft and thus guide the direction of shaft 2802. The stylet can start bending inside the lumen of the device due to its inherent shape memory effect. Rotating the stylet so that it points in the desired direction can deflect the tip in the desired direction, thus directing a guidewire 2801 in the same direction as well. Accordingly, the distal end of the device can continue to be inserted until it reaches the occlusion now that it has traversed the branching point due to the stylet.

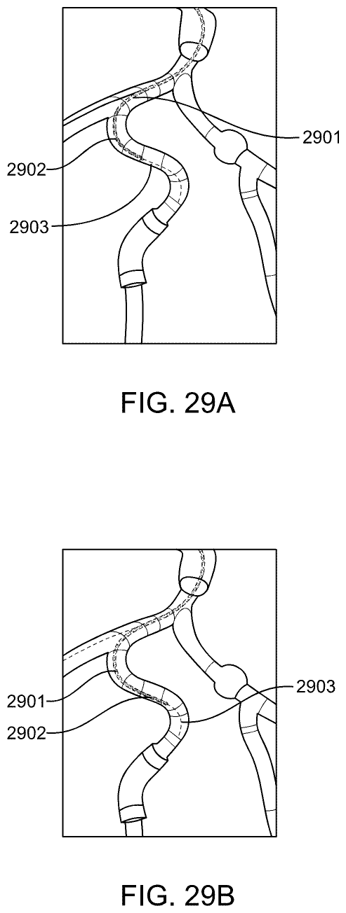

In some embodiments, inserting can comprise extending the device to a plaque and/or occlusion mass of interest via a telescoping version of one of the devices disclosed herein. For example, devices 10, 600, 620, 700, 800, or any of the other crossing devices disclosed herein can be altered or manufactured to contain a telescoping feature as shown in FIG. 8 and described above. A device with a telescoping feature as in FIG. 8 may use the telescoping feature to maneuver past bends and branching points in the vessels, similar to a stylet or guidewire. This is shown in FIGS. 29A-29B and FIGS. 30A-30C.

In some cases, for example FIGS. 29A-29B, the device comprising a telescoping feature may remain in its telescoped form for an extended time while traversing the vessels until it reaches the plaque and/or occlusion mass of interest and/or no longer needs to be in the telescoping form. FIG. 29A shows outer shaft 2901, inner shaft 2902, and guidewire 2903. The bundle between the inner shaft 2902 and the guidewire 2903 is the tip with cutting loop. FIG. 29A shows these three parts of the device after the inner shaft 2902 has followed the guidewire 2903 through the branching and bending point but before the outer shaft 2901 has followed the guidewire 2903 and inner shaft 2902.

FIG. 29B shows the same three elements—outer shaft 2901, inner shaft 2902, and guidewire 2903—but after the outer shaft 2901 has followed the guidewire 2903 and inner shaft 2902 past the bend. Together, these two figures show that telescoping can be a successful method to navigate past bends and branching points in the vessels, similar to a stylet or guidewire. This method can be used using any of the handles shown in FIGS. 2A-2C.

The length of the guidewire in front of the tip can vary. In some cases, for example when traversing bends and branch points, it can be beneficial to have the guidewire extended further from the tip and the rest of the device. In some cases, as shown in FIGS. 29A-29B, the guidewire can maintain a fairly consistent distance from the tip for consistent guidance through the vessels. In some cases, having the guidewire in front of the tip and rest of the device, even if not substantially in front, may help to minimize injury to the vessel wall. This can occur because the guidewire encounters the vessel wall first and can rebound or adjust movement based on that contact, and the guidewire can be less damaging than the device tip. In some cases, for example when the device has arrived at the site of the occlusion, the guidewire can be withdrawn into the device lumen to allow the tip and cutting loop(s) to cut through the occlusion.

In some cases, for example FIGS. 30A-30C, the device comprising a telescoping feature may temporarily use its telescoped form to traverse a bend or branch point before returning to its non- or less telescoped form by having the outer shaft “catch up” to the inner shaft. FIG. 30A shows outer shaft 3001, inner shaft 3002, and guidewire 3003. The bundle between the inner shaft 3002 and the guidewire 3003 is the tip with cutting loop. FIG. 30A shows these three parts of the device before the inner shaft 3002 has followed the guidewire 3003 through the branching and bending point but before the outer shaft 3001 has followed the guidewire 3003 and inner shaft 3002. FIG. 30B shows the same three elements—outer shaft 3001, inner shaft 3002, and guidewire 3003—after the inner shaft 3002 has followed the guidewire 3003 but before the outer shaft 3001 has followed the inner shaft 3002 and guidewire 3003. FIG. 30C shows the same three elements—outer shaft 3001, inner shaft 3002, and guidewire 3003—after the outer shaft 3001 has followed the inner shaft 3002 and guidewire 3003 past the bend.

Although the visible length of the inner shaft 3002 is similar between FIG. 30A and FIG. 30B, it is shorter in FIG. 30C. This is because the inner shaft 3002 and tip have been kept roughly steady while moving the outer shaft 3001 towards them. In this way, the outer shaft 3001 can “catch up” to the inner shaft 3002 and guidewire 3003. In some cases, once caught up, the outer shaft 3001 can cover the entirety of the inner shaft 3002 and only leave the tip. In some cases, once caught up, the outer shaft 3001 can leave some of the inner shaft in a telescoped form, as shown in FIG. 30C.

The benefit of the ability to adjust the outer shaft 3001 to be near the tip and guidewire is that the outer shaft 3001 can provide more support to both the inner shaft 3002 and guidewire 3003. The outer shaft 3001 can act like a support microcatheter for the elongated or telescoped inner shaft 3002. This can allow the device to exert more force when encountering plaque and/or occlusion mass regions.

As the insertion method shown in FIGS. 30A-30C may benefit from independent motion of the outer shaft 3001 and the inner shaft 3002, the handle in FIG. 2C may be appropriate, as that handle has a separate movement tab for the outer shaft.

The length of the guidewire in front of the tip can vary. In some cases, for example when traversing bends and branch points, it can be beneficial to have the guidewire extended further from the tip and the rest of the device. In some cases, as shown in FIGS. 30A-30B, the guidewire can maintain a fairly consistent distance from the tip for consistent guidance through the vessels. In some cases, having the guidewire in front of the tip and rest of the device, even if not substantially in front, may help to minimize injury to the vessel wall. This can occur because the guidewire encounters the vessel wall first and can rebound or adjust movement based on that contact, and the guidewire can be less damaging than the device tip. In some cases, for example when the device has arrived at the site of the occlusion, the guidewire can be withdrawn into the device lumen to allow the tip and cutting loop(s) to cut through the occlusion.

In some embodiments, inserting can comprise extending the device to a plaque and/or occlusion mass of interest via a guiding catheter. In some cases, coronary guiding catheters can be to access a patient's coronary arteries via the coronary ostia to provide access for subsequent interventional and diagnostic catheters to perform a variety of procedures, such as angioplasty, atherectomy, thrombectomy, intravascular ultrasound (IVUS), and the like. In some cases, the interventional catheters comprise any of the devices described herein. In some cases, the interventional catheters comprise a plurality of the devices described herein. The guiding catheters can comprise an elongated catheter body such as a tubular member with a proximal and distal end and a central lumen running along its length. The outer diameters may range 5 F to 8 F (1 F (French) equals about 0.33 mm) and the length may range from about 90 cm to about 110 cm. The guiding catheters can be used for femoral access and radial access in coronary interventions using the coronary interventional devices described herein.

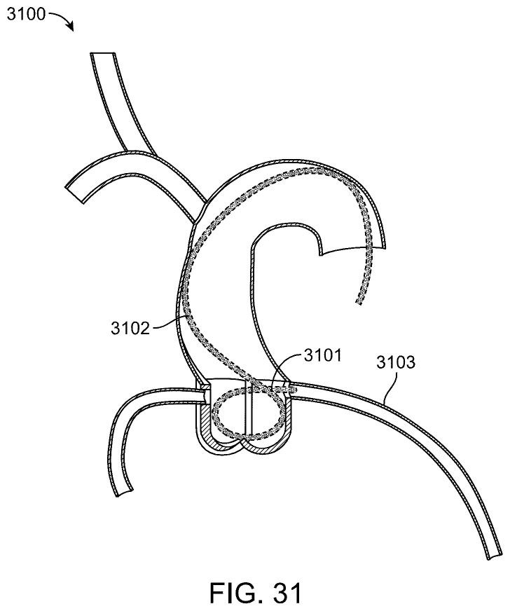

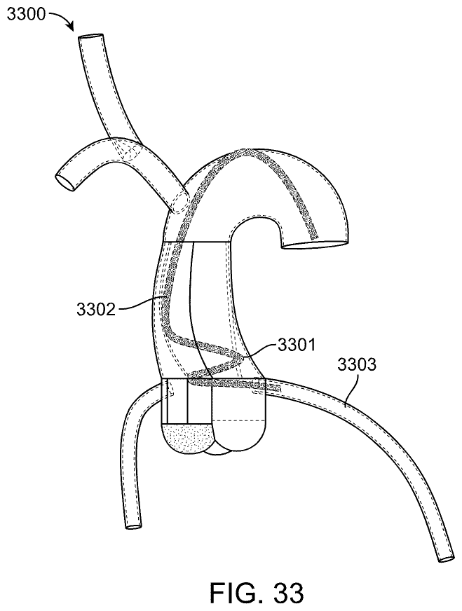

In some embodiments, a guiding catheter can be improved by increasing stability, thereby acting as a coronary stabilization system. In some embodiments, the stability of the guiding catheter is derived from its unique pre-set ellipsoidal shape at its distal segment. FIG. 31 shows a side view of a coronary stabilization method with a low loop comprising a pre-set ellipsoidal shape in the aorta in accordance with methods described herein. The ellipsoidal shape can have the largest possible radius of curvature at any point on its curvature while retaining the tip's co-axial orientation with the coronary ostium at baseline as well as during axial push application on the inlaying interventional device by the user. In addition, the guiding catheter's recurved, ellipsoidal-shaped distal segment, snugly buttresses against wall of the aortic root. The dimensions of this ellipsoidal segment are from 2 cm to 2.5 cm vertically and 3 cm to 4 cm horizontally, so as to accommodate into lumen of the normative aortic sinus. In some cases, the dimensions are swapped, such as when the ellipsoidal segment is rotated 90 degrees to lie horizontally, as in FIG. 33. In some cases, the dimensions are less than 2 cm. In some cases, the dimensions are greater than 4 cm. Grades of larger and smaller sizes over this size can also be available to accommodate aortic roots of various sizes.

The recurved ellipsoidal working end can provide multiple advantages, which together considerably can add up to the system's stability, efficacy, and safety. First, a large contact surface area of the guiding catheter can meet with the adjoining aortic sinus and aortic valve at rest and during an inlaying device passage. The ellipsoidal shape at the working distal end can impart stability to the guiding catheter as it snugly fits inside a U-shaped anatomical area formed by the aortic sinus and aortic valve resulting in a large contact area. This large contact area between the guiding catheter and aortic sinus/aortic valve can provide a large static friction, which in turn boosts the resistance to guiding catheter's backup tendency.

While crossing a lesion with a device, a user can encounter guiding catheter/device backout, resulting in loss of stability to various degrees. To counter this backout, the user may apply forward counter-push on the guiding catheter shaft, which further inadvertently adds up to destabilization of the guiding catheter. In the present disclosure, the backup movement of a guiding catheter as a reaction to the inlaying interventional device forward push can be effectively counterbalanced by the forward axial push on the guiding catheter shaft by the user due to the ellipsoidal segment being further snugly buttressing up against the aortic wall, thus resulting in minimal net movement of the guiding catheter and its tip from the ostial location.

In some embodiments, a further advantage of the guiding catheters disclosed herein is the minimal possible contact area between outer surface of the inlying device with the inner surface of the guiding catheter at every point on the ellipsoidal segment. The ellipsoidal shape may be unchanged even with the interventional device advancement over a much bigger range of axial force application on the interventional device as it is buttressed against the aortic sinus and valve.

In some embodiments, the shape minimizes the potential loss of axial forces. A sharper bend may result in a higher net force loss as the angle increases from about 0° to about 90°. In contrast, a smooth curve, as shown in FIG. 31, consisting of an infinitesimal number of sequentially placed bends with minute angles together results in a much smaller loss of the net axial force when compared to a single sharp bend greater than or equal to 45°.

Furthermore, even if the guiding catheter tip is partially backed out of the coronary ostia during advancement of the inlaying interventional device through a tight coronary lesion or hard occlusion, the straighter distal segment of the preset invention can maintain a co-axial orientation with the proximal coronary artery and its ostium. Thus, the guiding catheters disclosed herein can withstand a high axial displacement and force before losing their stability. This can successfully stabilize the interventional device when crossing a harder and tighter lesion which would otherwise get destabilized. This can also enable any interventional devices described herein to steer through hard stiff calcified tortuous coronaries, as well as acute coronary branch points, a situation where these devices may otherwise fail due to destabilization and disengagement.

The guiding catheters and insertion methods described herein can also enhance the safety of each coronary interventional procedure. Inadequate guiding catheter support during a coronary intervention can result in loss guiding catheter stability. The need to realign and reengage the guiding catheter is associated with the risk of coronary ostial dissection, acute coronary thrombosis and myocardial infarction. The stable guiding catheter of the present disclosure can reduce such risks. In addition, procedures using the guiding catheter can be shorter, reducing both radiation and contrast dosages.

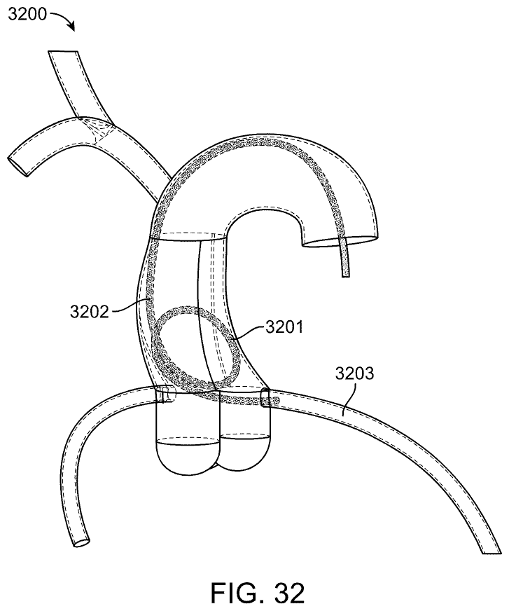

FIG. 32 shows a side view of the coronary stabilization method with a high loop in the aorta in accordance with methods described herein. The ellipsoidal loop at the distal end of the guiding catheter can be disposed at a higher level, just above the coronary ostial plane in a non-interfering position with the aortic valve movement. In some cases, a guiding catheter induced aortic insufficiency during coronary interventions can occur, which can potentially lead to transient hemodynamic instability. The noninterfering position of the guiding catheter loop in this embodiment can avoid this risk.

The ellipsoidal shape of the guiding catheter may provide resilience and can withstand deformation forces resulting from guide catheter/interventional device manipulations during coronary interventions. This performance characteristic can be improved by modifying mechanical properties of the shaft, such as modifying the properties of a stainless-steel braid reinforcement used during guide catheter extrusion. This can be done by adjusting the braid wire's thickness, cross section geometry, material composition, pitch ratio of the mesh, and/or by adding a second braid made up of memory shape metal alloy (such as nitinol) at the ellipsoidal segment.

In some cases, another advantage of the present guiding catheter derived from its unique ellipsoidal shape is the ability to accommodate many different locations and orientations of coronary ostia. This can be done by advancing and/or withdrawing the guiding catheter until its tip get oriented co-axially with respect to the target coronary ostia, and thus allowing the ellipsoidal segment to get tightly engaged the aortic wall. For example, FIG. 33 shows a side view of the coronary stabilization method with a horizontal loop in the aorta in accordance with methods described herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

It should be noted that there may be at least one or two possible types of rings in the devices described herein. In some cases, the devices described herein may comprise a “C ring” or a “retaining ring,” as described above with the functions described above. These two names may be synonymous because “C ring” can describe the shape while “retaining ring” can describe the functionality. In some cases, the devices described herein may comprise an “outer member” ring or “OM” ring. This second type of ring may encircle the outer shaft.

It should be noted that “create” and “enlarged” are used throughout the specification in relation to the effect of the devices and methods described herein on channel formation or expansion.

However, these can be used interchangeably, as “create” can apply to partial occlusions as well as chronic total occlusions and “expand” can apply to channels that are so small that there is zero, or substantially zero, blood flow through them.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The ranges disclosed herein also encompass any and all overlap, sub-ranges, and combinations thereof. Language such as “up to,” “at least,” “greater than,” “less than,” “between,” “from,” and the like includes the number recited. Numbers preceded by a term such as “approximately”, “about”, and “substantially” as used herein include the recited numbers, and also represent an amount close to the stated amount that still performs a desired function or achieves a desired result. The term “about” or “approximately” may mean within an acceptable error range for the particular value, which will depend in part on how the value is measured or determined, e.g., the limitations of the measurement system. For example, the terms “approximately”, “about”, and “substantially” may refer to an amount that is within less than 10% of, within less than 5% of, within less than 1% of, within less than 0.1% of, and within less than 0.01% of the stated amount. For example, “about” may mean within 1 or more than 1 standard deviation, per the practice in the art. Alternatively, “about” may mean a range of up to 20%, up to 10%, up to 5%, or up to 1% of a given value. As used herein, the term “about” a number refers to that number plus or minus 10% of that number. The term “about” a range refers to that range minus 10% of its lowest value and plus 10% of its greatest value. Where particular values are described in the application and claims, unless otherwise stated the term “about” meaning within an acceptable error range for the particular value may be assumed.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa.

The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### CLAUSES

Listed herein is a set of clauses providing the devices, systems, and methods described herein.
Clause 1. A method for treating an occluded blood vessel, the method comprising: centering a working end of a treatment catheter in the occluded blood vessel, wherein centering comprises maintaining the position of the working end within a lumen of the occluded blood vessel; disrupting the occlusion mass with the working end of the treatment catheter; radially displacing the disrupted occlusion mass and adjacent occlusion mass against a wall of the occluded blood vessel; wherein radially displacing the disrupted and adjacent occlusion mass creates a blood flow channel in the occlusion mass while simultaneously maintaining the centering of the working end of the treatment catheter, wherein radially displacing the disrupted and adjacent occlusion mass enlarges a diameter of the channel by compressing the disrupted and adjacent occlusion mass against a wall of the blood vessel while minimizing injury to the blood vessel wall; and withdrawing the treatment catheter from the blood vessel after the channel is enlarged, wherein the channel remains enlarged after the catheter is withdrawn.
Clause 2. The method of clause 1, wherein centering the working end of the catheter comprises centering the working end within a blood vessel wall.
Clause 3. The method of clause 1 or 2, wherein centering the working end of the catheter comprises deploying at least one flat spring laterally outwardly to atraumatically engage a wide lateral surface of the at least one flat spring against a wall region of the blood vessel to centrally align the catheter in a lumen of the blood vessel as the catheter is advanced.
Clause 4. The method of clause 3, wherein disrupting the occlusion mass comprises penetrating a narrow distal edge on the at least one flat spring through the occlusion to enlarge the channel through the occlusion as the catheter is advanced.
Clause 5. The method of any one of clauses 3 to 4, wherein radially displacing the disrupted and adjacent occlusion mass comprises self-deploying the at least one flat spring from a distal portion of the catheter after releasing the at least one flat spring from radial constraint so that the at least one flat spring elastically self-expands, wherein the at least one flat spring comprises at least one spiral flat spring.
Clause 6. The method of any one of clauses 3 to 5, wherein the at least one flat spring comprises a plurality of flat springs.
Clause 7. The method of any one of clauses 1 to 6, wherein centering the working end comprises deploying a centering element of the treatment catheter.
Clause 8. The method of clause 7, further comprising advancing the treatment catheter through the occluded blood vessel, wherein the deployed centering element radially displaces the disrupted occlusion mass as the treatment catheter is advanced through the occluded blood vessel.
Clause 9. The method of clause 7 or 8, wherein the centering element is positioned at or near the working end of the treatment catheter.
Clause 10. The method of any one of clauses 7 to 9, wherein the centering element comprises an expandable anchoring balloon.
Clause 11. The method of any one of clauses 7 to 10, wherein the centering element comprises at least one flat spring disposed circumferentially about a distal portion of a tubular catheter body of the treatment catheter and adapted to elastically self-expand from a radially constrained configuration to a radially expanded configuration wherein the at least one flat spring has a wide lateral surface configured to atraumatically engage against a wall region of the blood vessel to centrally align the tubular catheter body in a lumen of the blood vessel.
Clause 12. The method of clause 11, wherein at least one flat spring comprises three spiral flat springs spaced circumferentially apart by 120°.
Clause 13. The method of clause 11 or 12, wherein at least one flat spring has an Ω-shaped profile with a base portion attached to the tubular catheter body and a loop portion extending radially away from the tubular catheter body.
Clause 14. The method of any one of clauses 7 to 13, wherein the centering element comprises a cutting tip, at least one cutting loop, or both.
Clause 15. The method of clause 14, wherein centering comprises oscillating or rotating the cutting tip, at least one cutting loop, or both.
Clause 16. The method of clause 14 or 15, wherein the at least one cutting loop comprises two or more parallel cutting loops, two or more crossed cutting loops, at least one centered cutting loop, at least one cutting loop curved to a side, or any combination thereof.
Clause 17. The method of any one of clauses 14 to 16, wherein the cutting tip comprises a fluted tip, a pointed-end tip, a serrated tip, a helical screw tip, a tubular tip, a cone tip, a screw surface tip, an eyelet tip comprising a guidewire, or any combination thereof.
Clause 18. The method of any one of clauses 1 to 17, wherein disrupting the occlusion mass with the working end of the treatment catheter comprises cutting the occlusion mass with a cutting element of the working end of the treatment catheter.
Clause 19. The method of clause 18, wherein cutting the occlusion mass with the cutting element comprises rotating or oscillating the cutting element.
Clause 20. The method of clause 18 or 19, wherein centering the working end of the catheter comprises oscillating the cutting element.
Clause 21. The method of any one of clauses 18 to 20, wherein rotating or oscillating the cutting element comprises manually rotating or oscillating the cutting element.
Clause 22. The method of clause 21, wherein manually rotating or rotationally oscillating the tip comprises rotating or oscillating a wheel on a handle attached to a proximal end of the catheter.
Clause 23. The method of clause 21 or 22, wherein the cutting element comprises a cutting tip, at least one cutting loop, or both.
Clause 24. The method of clause 23, wherein disrupting the occlusion mass comprises one or more of: pushing the cutting tip mounted on a distal end of the catheter, or rotating the cutting tip.
Clause 25. The method of clause 23 or 24, wherein the at least one cutting loop comprises two or more parallel cutting loops, two or more crossed cutting loops, at least one centered cutting loop, at least one cutting loop curved to a side, or any combination thereof.
Clause 26. The method of any one of clauses 23 to 25, wherein the cutting tip comprises a fluted tip, a pointed-end tip, a serrated tip, a helical screw tip, a tubular tip, a cone tip, a screw surface tip, an eyelet tip comprising a guidewire, or any combination thereof.
Clause 27. The method of any one of clauses 1 to 26, wherein withdrawing the catheter comprises withdrawing the catheter over a guidewire disposed in a lumen of the catheter, wherein the guidewire remains in the blood vessel lumen.
Clause 28. The method of clause 27, wherein withdrawing the catheter comprises withdrawing the catheter from a distal end of the occlusion mass after the catheter had been advanced fully through the occlusion mass.
Clause 29. The method of clause 27 or 28, wherein withdrawing the catheter comprises withdrawing the catheter from within the occlusion mass after the catheter had been advanced partially through the occlusion, further comprising advancing the guidewire through a remaining portion of the occlusion mass.
Clause 30. The method of any one of clauses 27 to 29, wherein withdrawing the catheter comprises withdrawing the catheter from a proximal face of the occlusion mass prior to being retracted, further comprising advancing the guidewire through the occlusion mass.
Clause 31. The method of any one of clauses 27 to 30, further comprising inserting a second device over the guidewire after the treatment catheter is withdrawn.
Clause 32. The method of any one of clauses 27 to 31, wherein the guidewire is a shape memory guidewire.
Clause 33. The method of clause 32, further comprising extending the guidewire past an end of the catheter into the blood vessel after radially displacing the occlusion mass, wherein extending the guidewire causes it to return to a shape memory form, wherein the shape memory form comprises an embolic protection device comprising a spiral cone filter and a distal coil
Clause 34. The method of clause 33, further comprising capturing debris released while disrupting the occlusion mass in the spiral cone filter.
Clause 35. The method of clause 34, further comprising inserting a guide catheter over the guidewire, wherein the guide catheter comprises an aspiration syringe attached to the distal end of the guide catheter; and aspirating the debris captured in the spiral cone filter.
Clause 36. The method of any one of clauses 1 to 35, wherein the catheter comprises an interventional catheter, a delivery catheter, a guide catheter, a microcatheter, an embolic protection device, or any combination thereof.
Clause 37. The method of any one of clauses 1 to 36, wherein disrupting the occlusion mass causes substantially no dissection, cutting, perforation, damage, injury, or any combination thereof occurs to the blood vessel.
Clause 38. The method of any one of clauses 1 to 37, wherein radially displacing the disrupted and adjacent occlusion mass causes substantially no expansion, widening, extension, stretching, abrasion, cutting, or any combination thereof occurs to the blood vessel.
Clause 39. The method of clause 38, wherein radially displacing the disrupted and adjacent occlusion mass causes substantially no expansion, widening, extension, stretching, abrasion, cutting, or any combination thereof due to a substantially negligible strain rate, substantially negligible lateral force application, and/or substantially negligible active application of lateral strain rate.
Clause 40. The method of any one of clauses 1 to 39, further comprising administering one or more medications to the blood vessel while radially displacing the disrupted and adjacent occlusion mass, while withdrawing the catheter, with a second device after withdrawing the catheter, or any combination thereof.
Clause 41. The method of any one of clauses 1 to 40, wherein disrupting the occlusion mass comprises applying one or more of mechanical, vibrational, ultrasound, thermal, cooling, cryo, or optical energy to the occlusion mass.
Clause 42. The method of any one of clauses 1 to 41, wherein radially displacing the disrupted and adjacent occlusion mass enlarges a diameter of the channel to approximately match a widest part of the working end of the treatment catheter.
Clause 43. The method of any one of clauses 1 to 42, wherein radially displacing the disrupted and adjacent occlusion mass enlarges a diameter of the channel to greater than a widest part of the working end of the treatment catheter.
Clause 44. The method of any one of clauses 1 to 43, wherein the disrupted and adjacent occlusion mass is radially displaced by a radial distance less than a radial displacement of an outer wall of the blood vessel as the disrupted and adjacent occlusion mass are radially displaced.
Clause 45. The method of any one of clauses 1 to 44, wherein radially displacing the disrupted and adjacent occlusion mass enlarges the channel in the occluded blood vessel by between about 0.5 mm and about 3.6 mm in diameter.
Clause 46. The method of any one of clauses 1 to 45, wherein radially displacing the disrupted and adjacent occlusion mass enlarges the channel in the occluded blood vessel by between about 0.6 mm² and about 15 mm² in area.
Clause 47. The method of any one of clauses 1 to 46, wherein radially displacing the disrupted and adjacent occlusion mass comprises creating a channel in the occlusion mass.
Clause 48. The method of clause 47, wherein creating a channel comprises creating a channel having a diameter of about 0.5 mm to about 5 mm.
Clause 49. The method of clause 47 or 48, wherein creating a channel comprises creating a channel in the occlusion mass having a cross-sectional area of about 2 mm² to about 18 mm².
Clause 50. The method of any one of clauses 1 to 49, further comprising navigating the catheter to the occluded blood vessel prior to or while simultaneously centering the working end of the catheter.
Clause 51. The method of clause 50, further comprising steering the catheter through bends, angles, branch points, or any combination thereof, wherein steering comprises using a steering stylet.
Clause 52. The method of clause 51, wherein the steering stylet comprises a pre-shaped memory material pre-set to an angle or curve to steer the catheter through bends, angles, branch points, or any combination thereof.
Clause 53. The method of any one of clauses 1 to 52, wherein the occluded blood vessel is a coronary artery, a femoral artery, brachial artery, radial artery, pedal artery, or a tibial artery.
Clause 54. The method of any one of clauses 1 to 53, wherein the enlargement of the channel improves channel diameter by about 1 mm to about 4 mm in a vessel size of from about 1.5 mm to about 15 mm.
Clause 55. The method of any one of clauses 1 to 54, wherein the enlargement of the channel provides percentage improvement in channel diameter from about 5% to about 80%.
Clause 56. The method of any one of clauses 1 to 55, wherein the occluded blood vessel has a chronic total occlusion.
Clause 57. A treatment catheter for treating an occluded blood vessel, the catheter comprising: a tubular catheter body having a distal end, a proximal end, and a central passage therethrough; a rotatable shaft extending through the central passage of the tubular catheter body and having a distal end, a proximal end, and a central lumen therethrough; a cutting tip mounted on the distal end of the rotatable shaft and configured to cut through occlusive material when rotated, pushed, or both; wherein the cutting tip has a passage contiguous with the central lumen of the rotatable shaft; at least one flat spring disposed circumferentially about a distal portion of the tubular catheter body and adapted to elastically self-expand from a radially constrained configuration to a radially expanded configuration wherein each flat spring has (1) a wide lateral surface configured to atraumatically engage against a wall region of the blood vessel to centrally align the tubular catheter body in a lumen of the blood vessel and (2) a narrow distal edge configured to penetrate the occlusion as the catheter is distally advanced; and a handle at the proximal end of the tubular catheter body.
Clause 58. The catheter of clause 57, wherein tubular catheter body has a length in the range from about 120 cm to about 190 cm and a diameter from 1 mm to 4 mm.
Clause 59. The catheter of clause 57 or 58, further comprising a manual rotation device attached to the rotatable shaft.
Clause 60. The catheter of clause 59, wherein the manual rotation device comprises a rotating cylinder in the handle.
Clause 61. The catheter of any one of clauses 57 to 60, wherein the cutting tip includes at least one cutting loop extending in a distal direction from a distal end of the cutting tip.
Clause 62. The catheter of clause 61, wherein the at least one cutting loop extending in a distal direction from the distal end of the cutting tip is radially offset from the passage in the cutting tip.
Clause 63. The catheter of clause 61 or 62, wherein the passage in the cutting tip is aligned with a central axis of the rotatable shaft and the at least one cutting loop is radially offset from the central axis.
Clause 64. The catheter of any one of clauses 61 to 63, wherein the at least one cutting loop is aligned with a central axis of the rotatable shaft and the passage in the cutting tip is radially offset from the central axis.
Clause 65. The catheter of any one of clauses 61 to 64, wherein the at least one cutting loop is aligned with a central axis of the rotatable shaft and the passage in the cutting tip is radially offset from the central axis.
Clause 66. The catheter of any one of clauses 61 to 65, wherein the at least one cutting loop is curved to a side.
Clause 67. The catheter of any one of clauses 61 to 66, wherein the cutting tip includes at least two cutting loops extending in a distal direction from a distal end of the cutting tip.
Clause 68. The catheter of clause 67, wherein the at least two cutting loops form crossed cutting loops, wherein the crossed cutting loops have at least three legs.
Clause 69. The catheter of any one of clauses 57 to 68, wherein the cutting tip comprises a fluted distal surface.
Clause 70. The catheter of any one of clauses 57 to 69, wherein the cutting tip comprises a pointed-end tip, a serrated tip, a helical screw tip, a tubular tip, a cone tip, a screw surface tip, an eyelet tip comprising a guidewire, or any combination thereof.
Clause 71. The catheter of any one of clauses 57 to 70, wherein each of the at least one flat spring has an Ω-shaped profile with a base portion attached to the tubular catheter body and a loop portion extending radially away from the tubular catheter body.
Clause 72. The catheter of any one of clauses 57 to 71, wherein the at least one flat spring is straight and tangential to the rotatable shaft.
Clause 73. The catheter of any one of clauses 57 to 72, wherein the at least one flat spring flat spring is configured to retract.
Clause 74. The catheter of clause 73, wherein the retraction can be actuated by a spring.
Clause 75. The catheter of any one of clauses 57 to 74, wherein the at least one flat spring extends from the distal portion of the tubular catheter body to beyond the cutting tip.
Clause 76. The catheter of any one of clauses 57 to 75, wherein the location of the at least one flat spring on the distal portion of the tubular catheter body is restricted by restraining tabs.
Clause 77. The catheter of any one of clauses 57 to 76, wherein the at least one flat spring comprises three spiral flat springs.
Clause 78. The catheter of clause 77, wherein the three spiral flat springs are spaced circumferentially apart by 120°.
Clause 79. The catheter of clause 57 to 78, further comprising at least a second flat spring distal to the at least one flat spring on the distal portion of the tubular catheter body, wherein the second flat spring is disposed on an inner shaft that telescopes the cutting tip.
Clause 80. The catheter of clause 79, wherein the at least a second flat spring comprises a plurality of flat springs.
Clause 81. The catheter of any one of clauses 57 to 80, wherein the catheter further comprises a guidewire disposed within the central lumen of the rotatable shaft.
Clause 82. The catheter of clause 81, wherein the guidewire comprises a coiled distal tip.
Clause 83. The catheter of clause 82, wherein the coiled distal tip comprises a pre-shaped memory material set to coil when advanced from the central lumen.
Clause 84. The catheter of clause 82 or 83, wherein the coiled distal tip comprises a flexible segment, a flexible coiled segment, a transitional segment, and a stiff segment; wherein the length of both flexible segments combined is approximately 15 mm.
Clause 85. The catheter of clause 84, wherein the coiled distal tip is configured to navigate past a bend, angle, branch point, or any combination thereof of the blood vessel by locating the transitional segment across the bend, angle, branch point, or any combination thereof.
Clause 86. The catheter of any one of clauses 57 to 85, further comprising a steering stylet.
Clause 87. The catheter of clause 86, wherein the steering stylet comprises a pre-shaped memory material.
Clause 88. The catheter of clause 87, wherein the steering stylet is configured to steer the cutting tip, rotatable shaft, or both in a direction imprinted into the pre-shaped memory material.
Clause 89. The catheter of clause 86 or 87, wherein the steering stylet comprises a stiffness greater than a stiffness of the rotatable shaft at a distal end of the rotatable shaft.
Clause 90. The catheter of any one of clauses 57 to 89, wherein the catheter is sized to enter at the femoral, radial, tibial, pedal, coronary, or brachial vessels.
Clause 91. A treatment catheter for treating an occluded blood vessel, the catheter comprising: a tubular catheter body having a distal end, a proximal end, and a central passage therethrough; a rotatable shaft extending through the central passage of the tubular catheter body and having a distal end, a proximal end, and a central lumen therethrough; a cutting tip mounted on the distal end of the rotatable shaft and configured to cut through occlusive material when rotated, pushed, or both; wherein the cutting tip has a passage contiguous with the central lumen of the rotatable shaft; and an expandable dilating element disposed circumferentially about a distal portion of the tubular catheter body distal to at least one centering and occlusion mass displacement element and configured to pre-dilate the occluded blood vessel before the at least one centering and occlusion mass displacement element engages the occluded blood vessel.
Clause 92. The catheter of clause 91, wherein the at least one centering and occlusion mass displacement element comprises at least one flat spring is disposed circumferentially about a distal portion of the tubular catheter body and adapted to elastically self-expand from a radially constrained configuration to a radially expanded configuration wherein each flat spring has (1) a wide lateral surface configured to atraumatically engage against a wall region of the blood vessel to centrally align the tubular catheter body in a lumen of the blood vessel and (2) a narrow distal edge configured to penetrate the occlusion as the catheter is distally advanced.
Clause 93. The catheter of clause 91 or 92, wherein the dilating element comprises an expandable balloon.
Clause 94. The catheter of any one of clauses 91 to 93, wherein the dilating element comprises a diameter of between about 2 mm and about 4 mm.
Clause 95. The catheter of any one of clauses 91 to 94, wherein the cutting tip comprises at least one cutting loop.
Clause 96. The catheter of clause 95, wherein the at least one cutting loop is radially offset from the passage in the cutting tip.
Clause 97. The catheter of clause 95 or 96, wherein the passage in the cutting tip is aligned with a central axis of the rotatable shaft and the at least one cutting loop is radially offset from the central axis.
Clause 98. The catheter of any one of clauses 95 to 97, wherein the at least one cutting loop is aligned with a central axis of the rotatable shaft and the passage in the cutting tip is radially offset from the central axis.
Clause 99. The catheter of any one of clauses 95 to 98, wherein the at least one cutting loop is aligned with a central axis of the rotatable shaft and the passage in the cutting tip is radially offset from the central axis.
Clause 100. The catheter of any one of clauses 95 to 99, wherein the at least one cutting loop is curved to a side.
Clause 101. The catheter of any one of clauses 95 to 100, wherein the at least one cutting loop comprises at least two cutting loops extending in a distal direction from a distal end of the cutting tip.
Clause 102. The catheter of clause 101, wherein the at least two cutting loops form crossed cutting loops, wherein the crossed cutting loops have at least three legs.
Clause 103. The catheter of any one of clauses 91 to 102, wherein the cutting tip comprises a fluted tip, pointed-end tip, a serrated tip, a helical screw tip, a tubular tip, a cone tip, a screw surface tip, an eyelet tip comprising a guidewire, or any combination thereof.
Clause 104. The catheter of any one of clauses 91 to 103, wherein the catheter further comprises a guidewire disposed within the central lumen of the rotatable shaft.
105. A treatment catheter for treating an occluded blood vessel, the catheter comprising: a tubular catheter body having a distal end, a proximal end, and a central passage therethrough; a rotatable shaft extending through the central passage of the tubular catheter body and having a distal end, a proximal end, and a central lumen therethrough; and an energy projecting element, wherein the energy projecting element is configured to disrupt occlusive material in the occluded blood vessel.
Clause The catheter of clause 105, wherein the energy projecting element disrupts the occlusive material prior to a radial displacement of the occlusive material.
Clause 107. The catheter of clause 105 or 106, wherein the energy projecting element is disposed on the tubular catheter body.
Clause 108. The catheter of any one of clauses 105 to 107, wherein the energy projecting element is disposed on the rotatable shaft.
Clause 109. The catheter of clause 108, wherein the energy projecting element is configured to disrupt the occlusive material while rotating.
Clause 110. The catheter of any one of clauses 105 to 109, wherein the energy projecting element is configured to disrupt the occlusive material by applying vibrational, ultrasound, thermal, cooling, cryo, or optical energy to the occlusive material.
Clause 111. The catheter of any one of clauses 105 to 110, further comprising at least one flat spring disposed circumferentially about a distal portion of the tubular catheter body and adapted to elastically self-expand from a radially constrained configuration to a radially expanded configuration wherein each flat spring has (1) a wide lateral surface configured to atraumatically engage against a wall region of the blood vessel to centrally align the tubular catheter body in a lumen of the blood vessel and (2) a narrow distal edge configured to penetrate the occlusion as the catheter is distally advanced.
Clause 112. A treatment catheter for treating an occluded blood vessel, the catheter comprising: a rotatable shaft having a distal end, a proximal end, and a central lumen therethrough; a cutting tip mounted on the distal end of the rotatable shaft and configured to cut through occlusive material when rotated, pushed, or both; at least one cutting loop mounted on a distal end of the cutting tip; wherein the cutting tip has a passage contiguous with the central lumen of the rotatable shaft; wherein the cutting tip and the at least one cutting loop are configured to centrally align the catheter in a lumen of the blood vessel; and a handle at the proximal end of the rotatable shaft.
Clause 113. The catheter of clause 112, wherein the catheter lacks a deployable centering mechanism.
Clause 114. The catheter of clause 112 or 113, wherein rotatable shaft has a length in the range from 120 cm to 160 cm and a diameter from about 1 mm to about 1.5 mm.
Clause 115. The catheter of any one of clauses 112 to 114, further comprising a manual rotation device attached to the rotatable shaft.
Clause 116. The catheter of clause 115, wherein the manual rotation device comprises a rotating cylinder in the handle.
Clause 117. The catheter of any one of clauses 112 to 116, wherein the at least one cutting loop extending is radially offset from the passage in the cutting tip.
Clause 118. The catheter of any one of clauses 112 to 117, wherein the passage in the cutting tip is aligned with a central axis of the rotatable shaft and the at least one cutting loop is radially offset from the central axis.
Clause 119. The catheter of any one of clauses 112 to 118, wherein the at least one cutting loop is aligned with a central axis of the rotatable shaft and the passage in the cutting tip is radially offset from the central axis.
Clause 120. The catheter of any one of clauses 112 to 119, wherein the at least one cutting loop is aligned with a central axis of the rotatable shaft and the passage in the cutting tip is radially offset from the central axis.
Clause 121. The catheter of any one of clauses 112 to 120, wherein the at least one cutting loop is curved to a side.
Clause 122. The catheter of any one of clauses 112 to 121, wherein the at least one cutting loop comprises at least two cutting loops extending in a distal direction from a distal end of the cutting tip.
Clause 123. The catheter of clause 122, wherein the at least two cutting loops form crossed cutting loops, wherein the crossed cutting loops have at least three legs.
Clause 124. The catheter of clause 122 or 123, wherein the at least two cutting loops form crossed cutting loops, wherein the crossed cutting loops have at least four legs.
Clause 125. The catheter of any one of clauses 112 to 124, wherein the cutting tip comprises a fluted distal surface.
Clause 126. The catheter of any one of clauses 112 to 125, wherein the cutting tip comprises a pointed-end tip, a serrated tip, a helical screw tip, a tubular tip, a cone tip, a screw surface tip, an eyelet tip comprising a guidewire, or any combination thereof.
Clause 127. The catheter of any one of clauses 112 to 126, further comprising a telescoping mechanism configured to telescope the cutting tip away from the rotatable shaft.
Clause 128. The catheter of clause 127, further comprising an expandable anchoring balloon disposed on the rotatable shaft proximal to the cutting tip.
Clause 129. The catheter of any one of clauses 112 to 128, further comprising an outer shaft with a second lumen, wherein the outer shaft is configured to encircle the rotatable shaft and the second lumen.
Clause 130. The catheter of clause 129, further comprising a first guidewire in the central lumen of the rotatable shaft and a second guidewire in the second lumen.
Clause 131. The catheter of clause 130, wherein the outer shaft has a distal exit hole for the second lumen that is proximal to the cutting tip.
Clause 132. The catheter of any one of clauses 112 to 131, wherein the catheter further comprises a guidewire disposed within the central lumen of the rotatable shaft.
Clause 133. The catheter of clause 132, wherein the guidewire comprises a coiled distal tip.
Clause 134. The catheter of clause 133, wherein the coiled distal tip comprises a pre-shaped memory material set to coil when advanced from the central lumen.
Clause 135. The catheter of clause 133 or 134, wherein the coiled distal tip comprises a flexible segment, a flexible coiled segment, a transitional segment, and a stiff segment; wherein the length of both flexible segments combined is approximately 15 mm.
Clause 136. The catheter of clause 135, wherein the coiled distal tip is configured to navigate past a bend, angle, branch point, or any combination thereof of the blood vessel by locating the transitional segment across the bend, angle, branch point, or any combination thereof.
Clause 137. The catheter of any one of clauses 112 to 136, further comprising a steering stylet.
Clause 138. The catheter of clause 137, wherein the steering stylet comprises a pre-shaped memory material.
Clause 139. The catheter of clause 138, wherein the steering stylet is configured to steer the cutting tip, rotatable shaft, or both in a direction imprinted into the pre-shaped memory material.
Clause 140. The catheter of any one of clauses 137 to 139, wherein the steering stylet comprises a stiffness greater than a stiffness of the rotatable shaft at a distal end of the rotatable shaft.
Clause 141. The catheter of any one of clauses 112 to 140, wherein the catheter is sized to enter at the femoral, radial, tibial, pedal, coronary, or brachial vessels.
Clause 142. A guiding catheter for accessing a target blood vessel in a patient's ascending aorta, the guiding catheter comprising: an elongated catheter body comprising a proximal end and a distal end and configured to slidably receive an interventional catheter through the proximal end and guide the interventional catheter to the target blood vessel; wherein a distal tip of the elongate catheter body is configured to removably engage an ostium of the target blood vessel; and a distal region of the elongate catheter body is formed as a loop with a curve that bends and crosses over itself.
Clause 143. The catheter of clause 142, wherein the elongated catheter body further comprises a generally straight proximal region joined to the distal region.
Clause 144. The catheter of clause 143, wherein the generally straight proximal region is configured to lie over an aortic arch when the distal tip engages the target blood vessel ostium.
Clause 145. The catheter of clause 143 or 144, wherein the generally straight proximal region is configured to pass through a radial artery when the distal tip engages the target blood vessel ostium.
Clause 146. The catheter of any one of clauses 143 to 145, wherein the distal region consists of a single loop that subtends an arc in a range from about 240° to about 300° between the generally straight proximal portion to the distal tip.
Clause 147. The catheter of any one of clauses 143 to 145, wherein the distal region consists of a single loop that subtends an arc in a range from about 350° to about 390° between the generally straight proximal portion to the distal tip.
Clause 148. The catheter of any one of clauses 142 to 147, wherein the looped distal region of the elongate catheter body is configured to radially expand within the ascending aorta to impinge against opposed walls of the patient's ascending aorta and to have a distal-most bend positioned over an aortic valve.
Clause 149. A method for accessing a target blood vessel in a patient's ascending aorta, the method comprising: advancing a distal region of an elongate catheter body into the patient's ascending aorta, wherein the distal region of the elongate catheter is constrained in a straightened configuration; releasing the distal region of the elongate catheter body from constraint, wherein the distal region forms a loop with a curve that bends and crosses over itself within the patient's ascending aorta; engaging a distal tip of the elongate catheter body against an ostium of the target blood vessel; and advancing an interventional catheter through a lumen of the elongate catheter body to the ostium and into the target blood vessel.
Clause 150. The method of clause 149, wherein the elongate catheter body is advanced over an aortic arch of the patient.
Clause 151. The method of clause 149 or 150, wherein the elongate catheter body is advanced through a radial artery of the patient.
Clause 152. The method of any one of clauses 149 to 151, wherein the elongate catheter body is advanced over a stiff guide wire and releasing the distal region of the elongate catheter body from constraint comprises withdrawing the stiff guide wire from the distal region.
Clause 153. The method of any one of clauses 149 to 152, wherein the distal region consists essentially of a single loop that subtends an arc in a range from about 240° to about 300° between a generally straight proximal portion to the distal tip.
Clause 154. The method of any one of clauses 149 to 153, wherein the distal tip is configured to engage a right or left coronary ostium.
Clause 155. The method of any one of clauses 149 to 152, wherein the distal region comprises a loop that subtends an arc in a range from about 350° to about 390° between a generally straight proximal portion to the distal tip.
Clause 156. The method of any one of clauses 149 to 155, wherein the loop comprises a vertical loop.
Clause 157. The method of clause 156, wherein the looped segment is disposed above the coronary ostial plane in a noninterfering position to the aortic valvular movement.
Clause 158. The method of clause 156 or 157, wherein the looped segment is disposed below the coronary ostial plane in a noninterfering position to the aortic valvular movement.
Clause 159. The method of any one of clauses 149 to 158, wherein the loop comprises a horizontal loop.
Clause 160. The method of any one of clauses 149 to 159, wherein the looped distal region of the elongate catheter body radially expands within the patient's ascending aorta to impinge against opposed walls of the patient's ascending aorta and to have a distal-most bend positioned over an aortic valve when released from constraint.
Clause 161. The method of any one of clauses 149 to 160, further comprising inserting the elongate catheter body into a lumen of a hybrid guide catheter prior to advancing the distal region of the elongate catheter body into the patient's ascending aorta.
Clause 162. The method of clause 161, wherein advancing the distal region of the elongate catheter body into the patient's ascending aorta comprises advancing the elongate catheter body through the lumen of the hybrid guide catheter.
Clause 163. The method of any one of clauses 149 to 162, wherein the interventional catheter comprises the treatment catheter of any one of clauses 57 to 141.
Clause 164. The method of any one of clauses 149 to 163, wherein the elongated catheter body comprises the guide catheter of any one of clauses 142 to 148.
Clause 165. A catheter for treating an occluded blood vessel, the catheter comprising: a rotatable shaft having a distal end, a proximal end, and a central lumen therethrough; a cutting tip mounted on the distal end of the rotatable shaft and configured to cut through occlusive material when rotated, pushed, or both; wherein the cutting tip has a passage contiguous with the central lumen of the rotatable shaft; and an expandable element disposed circumferentially about a distal portion of the rotatable shaft, wherein the expandable element is configured to be in a contracted state when the catheter is inserted into the blood vessel and in an expanded state when the catheter is cutting through the occlusive material, wherein the expandable element is configured to exert force to radially displace occlusion mass causing the occlusion substantially while minimizing distension of a wall of the blood vessel.
Clause 166. The catheter of clause 165, wherein the expandable element is made at least partially of nitinol or another shape memory material.
Clause 167. The catheter of clause 165 or 166, further comprising an actuator for expanding the expandable element.
Clause 168. The catheter of any one of clauses 165 to 167, wherein the expandable element exerts a pressure on the occlusion mass being radially displaced.
Clause 169. The catheter of clause 168, wherein the pressure is less than about 2 mm Hg.
Clause 170. The catheter of any one of clauses 165 to 169, wherein the expandable element comprises an expandable balloon.
Clause 171. The catheter of clause 170, wherein the expandable balloon comprises an uneven balloon having a wider proximal end than distal end.
Clause 172. The catheter of clause 171, wherein a diameter at the proximal end is between about 3 mm and about 7 mm when expanded.
Clause 173. The catheter of any one of clauses 165 to 172, wherein minimizing distension of the wall of the blood vessel comprises increasing a diameter of the blood vessel at a location of the occlusive material by less than about 3 mm.
Clause 174. The catheter of any one of clauses 165 to 173, wherein the expandable element comprises a mechanically expanded cage.
Clause 175. The catheter of any one of clauses 165 to 174, wherein rotatable shaft has a length in the range from 120 cm to 160 cm and a diameter from about 1 mm to about 1.5 mm.
Clause 176. The catheter of any one of clauses 165 to 175, wherein the cutting tip comprises at least one cutting loop.
Clause 177. The catheter of clause 176, wherein the at least one cutting loop is radially offset from the passage in the cutting tip.
Clause 178. The catheter of clause 176 or 177, wherein the passage in the cutting tip is aligned with a central axis of the rotatable shaft and the at least one cutting loop is radially offset from the central axis.
Clause 179. The catheter of any one of clauses 176 to 178, wherein the at least one cutting loop is aligned with a central axis of the rotatable shaft and the passage in the cutting tip is radially offset from the central axis.
Clause 180. The catheter of any one of clauses 176 to 179, wherein the at least one cutting loop is aligned with a central axis of the rotatable shaft and the passage in the cutting tip is radially offset from the central axis.
Clause 181. The catheter of any one of clauses 176 to 180, wherein the at least one cutting loop is curved to a side.
Clause 182. The catheter of any one of clauses 176 to 181, wherein the at least one cutting loop comprises at least two cutting loops extending in a distal direction from a distal end of the cutting tip.
Clause 183. The catheter of clause 182, wherein the at least two cutting loops form crossed cutting loops, wherein the crossed cutting loops have at least three legs.
Clause 184. The catheter of clause 182 or 183, wherein the at least two cutting loops form crossed cutting loops, wherein the crossed cutting loops have at least four legs.
Clause 185. The catheter of any one of clauses 165 to 184, wherein the cutting tip comprises a fluted tip, pointed-end tip, a serrated tip, a helical screw tip, a tubular tip, a cone tip, a screw surface tip, an eyelet tip comprising a guidewire, or any combination thereof.
Clause 186. The catheter of any one of clauses 165 to 185, wherein the catheter further comprises a telescoping feature configured to telescope the cutting tip away from the rotatable shaft.
Clause 187. The catheter of any one of clauses 165 to 186, wherein the catheter further comprises a guidewire disposed within the central lumen of the rotatable shaft.
Clause 188. The catheter of clause 187, wherein the guidewire comprises a coiled distal tip.
Clause 189. The catheter of clause 188, wherein the coiled distal tip comprises a pre-shaped memory material set to coil when advanced from the central lumen.
Clause 190. The catheter of clause 188 or 189, wherein the coiled distal tip comprises a flexible segment, a flexible coiled segment, a transitional segment, and a stiff segment; wherein the length of both flexible segments combined is approximately 15 mm.
Clause 191. The catheter of clause 190, wherein the coiled distal tip is configured to navigate past a bend, angle, branch point, or any combination thereof of the blood vessel by locating the transitional segment across the bend, angle, branch point, or any combination thereof.
Clause 192. The catheter of any one of clauses 165 to 191, further comprising a steering stylet.
Clause 193. The catheter of clause 192, wherein the steering stylet comprises a pre-shaped memory material.
Clause 194. The catheter of clause 193, wherein the steering stylet is configured to steer the cutting tip, rotatable shaft, or both in a direction imprinted into the pre-shaped memory material.
Clause 195. The catheter of any one of clauses 192 to 194, wherein the steering stylet comprises a stiffness greater than a stiffness of the rotatable shaft at a distal end of the rotatable shaft.
Clause 196. The catheter of any one of clauses 165 to 195, wherein the catheter is sized to enter at the femoral, radial, tibial, pedal, coronary, or brachial vessels.
Clause 197. A catheter for treating an occluded blood vessel, the catheter comprising: a tubular catheter body having a distal end, a proximal end, and a central passage therethrough; a rotatable shaft extending through the central passage of the tubular catheter body and having a distal end, a proximal end, and a central lumen therethrough; a conical cutting tip mounted on the distal end of the rotatable shaft and configured to cut through occlusive material when rotated, pushed, or both, wherein the cutting tip comprises a helically cut surface; wherein the cutting tip has a passage contiguous with the central lumen of the rotatable shaft; a handle at the proximal end of the tubular catheter body.
Clause 198. A system for treating an occluded blood vessel, the system comprising: a treatment catheter, wherein the catheter comprises the catheter of any of clauses 57 to 141; a guidewire disposed inside the central lumen of the treatment catheter, wherein a distal end of the guidewire comprises a spiral cone filter configured to capture debris from crossing the occluded blood vessel, wherein a distal tip of the guidewire comprises a coil; and an aspiration syringe, wherein the aspiration syringe is configured to collect the captured debris from the spiral cone filter.
Clause 199. The system of clause 198, wherein the guidewire comprises a constrained state.
Clause 200. The system of clause 199, wherein the guidewire in the constrained state comprises a straight wire.
Clause 201. The system of any one of clauses 198 to 200, wherein the guidewire comprises a diameter between about 0.014″ to about 0.018″.
Clause 202. The system of any one of clauses 198 to 201, wherein the guidewire comprises a length of between about 180 cm to about 300 cm.
Clause 203. The system of any one of clauses 198 to 202, wherein the guidewire comprises a shape memory nitinol guidewire.
Clause 204. The system of any one of clauses 198 to 203, wherein the spiral cone filter comprises a length of between about 15 cm to about 20 cm.
Clause 205. The system of any one of clauses 198 to 204, wherein the spiral cone filter comprises a diameter of between about 7 mm to about 10 mm at a widest part of the spiral cone filter.
Clause 206. The system of any one of clauses 198 to 205, wherein the spiral cone filter comprises a height of between about 10 mm to about 15 mm.
Clause 207. The system of any one of clauses 198 to 206, wherein the spiral cone filter comprises a plurality of coils.
Clause 208. The system of clause 207, wherein a gap between each coil of the plurality of coils is less than 50 microns.
Clause 209. The system of clause 208, wherein the gap between each coil is sufficiently large that the spiral cone filter does not substantially interfere with passage of blood cells.
Clause 210. The system of any one of clauses 198 to 209, wherein the spiral cone filter comprises a polymer coating with positive electrostatic charges.
Clause 211. The system of any one of clauses 198 to 210, wherein the spiral cone filter comprises a coating of a PEGylated antibody to high-density lipoproteins.
Clause 212. The system of any one of clauses 198 to 211, wherein the spiral cone filter comprises a distal end and a proximal end, wherein the distal end is disposed about 5 cm proximal to an end of the guidewire.
Clause 213. The system of any one of clauses 198 to 212, wherein the coil is configured to protect a blood vessel wall from damage, injury, dissection, perfusion, or any combination thereof.
Clause 214. The system of any one of clauses 198 to 213, wherein a length of the coil is about 5 cm.
Clause 215. The system of any one of clauses 198 to 214, wherein 5 cm of the distal tip of the guidewire comprise the coil.
Clause 216. The system of any one of clauses 198 to 215, wherein the coil comprises a pig tail coil.
Clause 217. The system of any one of clauses 198 to 216, wherein the aspiration syringe comprises between about 30 cc to about 60 cc.
Clause 218. The system of any one of clauses 198 to 217, further comprising a guide catheter.
Clause 219. The system of clause 218, wherein the aspiration syringe is disposed on a distal end of the guide catheter.
Clause 220. The system of clause 218 or 219, wherein the guide catheter comprises a tip configured to be advance close to a tip of the spiral cone filter, wherein the aspiration syringe is configured to collect the captured debris from the tip of the spiral cone filter.
Clause 221. The system of any one of clauses 218 to 220, wherein the guide catheter has a diameter between about 2 mm to about 2.5 mm.
Clause 222. The method of any one of clauses 1 to 56, wherein disrupting the occlusion mass comprises pushing with a tip load force of from about 50 g to about 1500 g.

### EXAMPLES

The following examples are included for illustrative purposes only and are not intended to limit the scope of the present disclosure. Examples 1, 2, and 3 describe three studies that were conducted.

### Example 1: Safety and Effectiveness of the Peripheral CTO Crossing Device

A clinical trial was conducted on the safety and effectiveness of the peripheral CTO crossing devices described herein. In particular, the device used for this study was the device of FIGS. 3A-3B for crossing of chronic total occlusions (CTOs) in the peripheral arteries of lower limbs. The occlusions were located in the femoropopliteal arteries. The device was tested on approximately 74 individuals who were monitored over a period of about 30 days. The methods used were those shown in FIGS. 26A-26C and described herein.

The general inclusion criteria included being at least 22 years of age, having peripheral arterial disease as defined by Rutherford Clinical Classification (Category 2-5), and having the peripheral artery disease in the target extremity confirmed by imaging (e.g., catheter angiography, computed tomographic angiography (CTA), and/or magnetic resonance angiography (MRA)).

The angiographic inclusion criteria included the target lesion being located in the common femoral artery (CFA), superficial femoral artery (SFA), and/or popliteal artery; having vessel diameter(s) of the target lesion(s) from 3 mm and 10 mm; the target lesion having a severely stenosed segment of at most 300 mm that involves the CTO(s), the target lesion having at least one CTO that is 99-100% stenosed, and the subject having at least one vessel with run-off to the foot. Over 70% of the patients had moderate to severe calcification.

There were a number of exclusion criteria, such as infection, planned intervention during the study, bleeding diseases, allergies to the materials used, recent myocardial infarction or stroke, prior procedures or amputations on the limb, and various other criteria.

Clinical success was defined as Technical Success in the absence of device-related major adverse events through discharge or 24-hours post-procedure, whichever is sooner. Technical Success was defined as the ability of catheter to facilitate placement of a guidewire into the distal lumen without a procedural complication within 30 days after the procedure. Procedural complication was defined as the need for open or repeat endovascular surgical repair in the treated limb, or a major bleeding event.

Results: In the RESTOR-1 prospective multicenter pivotal study involving 74 study subjects, the CTO crossing device demonstrated 91.6% clinical success rate in per protocol subjects with no device related major adverse limb events. The device created a wide angiographic intraluminal channel in femoropopliteal arteries (2.87 mm mean diameter and 8.01 mm² mean cross sectional area) in every subject with technical success.

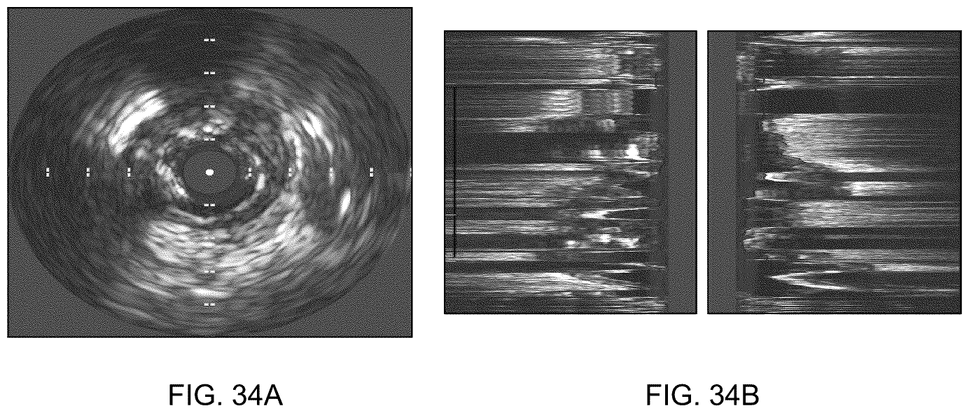

An example of the resulting channels is shown in FIGS. 34A-34B, as taken via IVUS and presented using a post processing software. Some of the tracts made were larger in diameter than the largest diameter of the device. Surprisingly, the vessels where the tracts were made showed zero, or negligible, distension, expansion, widening, extension, or stretching, abrasion, or cutting. Also surprising was that the device showed positive results (e.g., tract widening) in soft, medium, and hard calcification plaque, unlike atherectomy and angioplasty devices that specialize in hard plaque.

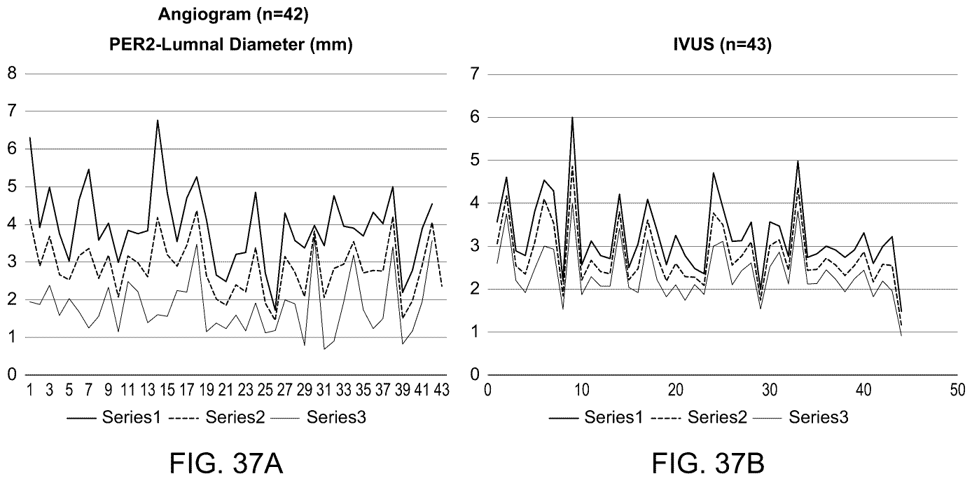

FIGS. 37A-37B illustrate graphs of channel diameters resulting from passage of a device as described herein as it crossed through an occlusion using methods described in example 1 and captured using an angiogram or an intravascular ultrasound (IVUS), respectively. The results of 42 patients are represented on the angiogram and the results of 43 patients (with available data) are represented on the IVUS. These represent the x-axis. The y-axis represents the tract diameter after use of a device described herein. The darkest line illustrates the maximum value results of each subject across three trials. The medium gray line illustrates the mean value results of each subject across three trials. The lightest line illustrates the minimum value results of each subject across the three trials.

As shown in FIG. 37A, across all of the participants in the trials of example 1, the single largest area of tract diameter based on the angiogram was 6.76 mm. The single smallest area of tract diameter based on the angiogram was 0.68 mm. Per the angiogram, the average of the areas with the largest tract diameters was 3.93 mm. Per the angiogram, the average of the areas with the mean value results of the tract diameters was 2.87 mm. Per the angiogram, the average of the areas with the smallest tract diameters was 1.80 mm.

As shown in FIG. 37B, across all of the participants in the trials of example 1, the single largest area of tract diameter based on the IVUS was 6 mm. The single smallest area of tract diameter based on the IVUS was 0.91 mm. Per the IVUS, the average of the areas with the largest tract diameters was 3.24 mm. Per the IVUS, the average of the areas with the mean value results of the tract diameters was 2.78 mm. Per the IVUS, the average of the areas with the smallest tract diameters was 2.36 mm. The average cross-sectional areas of channels resulting from passage of a device as described herein as it crossed through an occlusion using methods described in example 1 and captured using an IVUS were across trial 6.71 mm² participants. The single largest average cross sectional area for an individual was 20.33 mm².

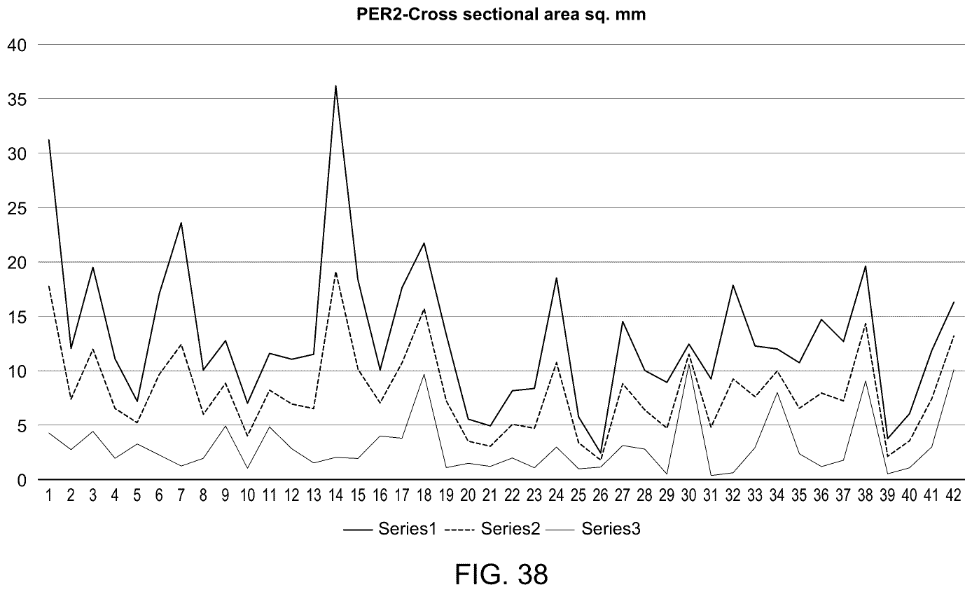

FIG. 38 illustrates a graph of the cross-sectional areas of channels resulting from passage of a device as described herein as it crossed through an occlusion using methods described in example 1 and captured using an angiogram. The graph was generated using a post-processing software. The x-axis represents the number of patients with available data (42). The y-axis represents the tract cross sectional area after use of a device described herein. The darkest line illustrates the maximum value results of each subject across three trials. The medium gray line illustrates the mean value results of each subject across three trials. The lightest line illustrates the minimum value results of each subject across the three trials.

As shown in FIG. 38, across all of the participants in the trials of example 1, the single largest area of tract cross sectional area based on the angiogram was 36.18 mm². The single smallest area of tract cross sectional area based on the angiogram was 0.49 mm². Per the angiogram, the average of the areas with the largest tract cross sectional areas was 12.98 mm². Per the angiogram, the average of the areas with the mean value results of the tract cross sectional areas was 8.01 mm².

Per the angiogram, the average of the areas with the smallest tract cross sectional areas was 3.04 mm².

### Example 2: Safety and Performance Study of Peripheral CTO Crossing Systems Above and Below the Knee

A clinical trial was conducted on the safety and effectiveness of the peripheral CTO crossing devices described herein. In particular, the device used for this study was the device of FIGS. 3A-3B for crossing of chronic total occlusions (CTOs) in the peripheral arteries of lower limbs. This study was for below the knee arteries and the femoropopliteal arteries.

### Objective

The goal was to demonstrate that the peripheral CTO Crossing Systems can safely and effectively provide guidewire access across chronic total occlusions in the peripheral arteries of lower limbs.

### Study Design

This was a prospective, non-randomized, multi-center study for treatment of chronic total occlusions found in the peripheral vasculature of the lower limbs. Patients were being enrolled and consented into the study based on history and prior diagnosis of peripheral CTO (e.g., symptoms, ultrasound, and angiography). Subjects were prepared, and medications were administered per the treatment protocol. An antegrade or retrograde femoral access was obtained using a 6 F or 7 F introducer sheath.

Baseline angiography of the target lesion was performed prior to placement of the device to determine CTO length, percent of stenosis, reference vessel diameter, target vessel tortuosity, and calcification severity.

The investigational device was prepped and positioned at the target lesion. The device was advanced across the proximal cap of the occlusion, the CTO body and up to 0.5-1 cm proximal to the distal cap of the CTO. The cutting loop of the device penetrated the distal cap. A guidewire was advanced across the penetrated distal cap and into the distal true lumen.

The peripheral device was removed and an Intravascular Ultrasound (IVUS) catheter was introduced over the guidewire, advanced in to the track made by the device passage, and intraluminal disposition of the track was recorded. The IVUS catheter was removed and the target lesion was treated either with a balloon, stent, and/or atherectomy, at the discretion of the operator.

Cine-angiography captured the entire procedure.

Patients were monitored overnight and followed through discharge and at 30 days and 6-months post procedure.

### Enrollment

Patients were enrolled in one site. Patients included men and women over 18 years of age with peripheral CTO lesions in the lower limbs having a Rutherford Clinical Classification Categories 2-5 and a life expectancy greater than 6 months at the time of enrollment.

There were a number of exclusion criteria, such as infection, planned intervention during the study, bleeding diseases, allergies to the materials used, recent myocardial infarction or stroke, prior procedures or amputations on the limb, and various other criteria.

### Inclusion Criteria

The following inclusion criteria were followed:
1. Subject was a suitable candidate for angiography and endovascular intervention per ESC guidelines I/TASC II recommendations.
2. Target lesion(s) that was in a native de novo common femoral (CFA), superficial femoral (SFA), popliteal and tibial arteries.
3. Peripheral Device Target lesion(s) reference vessel diameter was between ≥4 mm and ≤10 mm by visual estimate. Peripheral-2 Device Target lesion(s) reference vessel diameter was between ≥2.5 mm and ≤10.0 mm by visual estimate.
4. Peripheral Device Target lesion(s) length was ≤125 mm. Peripheral Device 2 Target lesion(s) length was ≤100 mm.
5. Target lesion(s) were CTO, 100% occluded per investigator visual assessment at time of procedure.
6. When the target CTO lesion was in the common, superficial femoral or popliteal arteries, subject had at least one patent tibial vessel on the target leg with run-off to the foot.
7. No evidence of aneurysm or dissection in target vessel.
8. Calcification of CTO region of lesion was less than severe.

### Outcome Measures

Safety: freedom from device-related Major Adverse Events (MAEs) at 30 days and 6 months post procedure, as per Clinical Events Committee (CEC) adjudication, defined as: cardiovascular related deaths, unplanned index limb amputation, dissection of grade C or greater that require an intervention to resolve, or symptomatic distal embolization, defined as clinical signs or symptoms of distal emboli detected in the treated limb distal to the treated lesion after the index procedure or noted angiographically after the index procedure and requiring mechanical or pharmacologic means to improve flow.

Performance/Technical Success: The ability of the catheter to intraluminally cross a CTO of the peripheral arteries of the lower limb and facilitate placement of a guidewire into the distal true vessel lumen, as assessed by IVUS and/or angiography via core lab evaluation.

Procedural Success: Achievement of Technical Success together with post-procedural patency. Post-procedural patency is defined as ≤50% residual percent diameter stenosis at the end of revascularization procedure and at 6 months. Additionally, the ability of the crossing catheter to perform as follows:
a) Pre-treatment assembly of the device over the guidewire and into the guide catheter without damage or kinking.
b) Delivery of the device, guidewire, and guide catheter assembly to the desired location proximal to the proximal cap of the CTO.
c) Extension and expansion of the Centering System out of the guide catheter.
d) Ability of the device to penetrate the proximal cap.
e) Ability of the cutting loop to rotate in response to the rotation of the handle wheel as it bores through the CTO.
f) Ability of the device tip/cutting loop to penetrate the distal cap.
g) Ability to place a guidewire in the true vessel lumen distal to the target CTO.
h) Ability to retrieve the device into the guide catheter.
i) Ability to remove the device from the body and maintain guidewire access.

### Data Analysis

Data was analyzed using statistical methods. Categorical data was summarized using frequency tables, presenting the subject counts and percentages. Continuous variables were summarized by the mean, standard deviation, median, minimum and maximum. Within-subject changes were analyzed parametrically using the Paired t-test if the differences are normally distributed, or non-parametrically using the Sign-Rank Test if the differences are not normally distributed.

Results: In the first in-human trial involving six subjects conducted in Paraguay and Panama, the peripheral crossing system demonstrated 75% technical success in crossing femoropopliteal and 100% technical success in tibial CTOs with no device-related adverse events. They created a wide intraluminal channel in all successful subjects without embolization risk.

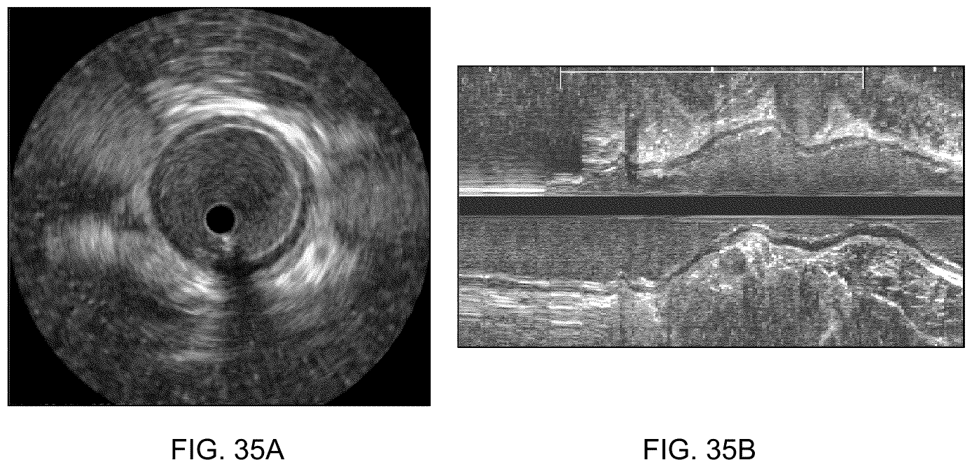

An example of the resulting channels is shown in FIGS. 35A-35B and FIGS. 1A-1B, as taken via IVUS and presented using a post processing software. Some of the tracts made were larger in diameter than the largest diameter of the device. Surprisingly, the vessels where the tracts were made showed zero, or negligible, distension, expansion, widening, extension, stretching, abrasion, or cutting. Also surprising was that the device showed positive results (e.g., tract widening) in soft, medium, and hard calcification plaque, unlike atherectomy and angioplasty devices that specialize in hard plaque.

The single largest area of tract diameter in the femoropopliteal region was 3.48 mm based on angiogram data. Based on IVUS data, it was 5.54 mm located in the below the knee region. The single smallest area of tract diameter was 0.96 mm in the below the knee region via angiogram data and 1.24 mm in the femoropopliteal region based on angiogram data. The average of the areas with the largest tract diameters was 3.05 mm via angiogram. The average of the areas with the mean value results of the tract diameters was 2.27 mm. The average of the areas with the smallest tract diameters was 1.51 mm.

The single largest area of tract cross sectional area based on the angiogram was 9.53 mm².

The single smallest area of tract cross sectional area based on the angiogram was 0.73 mm². Per the angiogram, the average of the areas with the largest tract cross sectional areas was 7.39 mm². Per the angiogram, the average of the areas with the mean value results of the tract cross sectional areas was 4.73 mm². Per the angiogram, the average of the areas with the smallest tract cross sectional areas was 2.06 mm². These included the femoropopliteal and below the knee results.

### Example 3: Safety and Performance Study of the Peripheral CTO Crossing System

A clinical trial was conducted on the safety and effectiveness of the peripheral CTO crossing devices described herein. In particular, the device used for this study was the device of FIGS. 3A-3B for crossing of chronic total occlusions (CTOs) in the peripheral arteries of lower limbs.

### Objective

The goal was to demonstrate that the peripheral CTO Crossing Systems can safely and effectively provide guidewire access across chronic total occlusions in the peripheral arteries of lower limbs.

### Study Design

This was a prospective, non-randomized, multi-center study for treatment of chronic total occlusions found in the peripheral vasculature of the lower limbs. Patients were enrolled and consented into the study based on history and prior diagnosis of peripheral CTO (e.g., symptoms, ultrasound, and angiography). Subjects were prepared, and medications were administered per the treatment protocol. A femoral or pedal access was obtained using a 6 F or 7 F introducer sheath.

Baseline angiography of the target lesion was performed prior to placement of the device to determine CTO length, percent of stenosis, reference vessel diameter, target vessel tortuosity, and calcification severity.

The investigational device was prepped and positioned at the target lesion. The device was advanced across the proximal cap of the occlusion, the CTO body and up to 0.5-1 cm proximal to the distal cap of the CTO. The cutting loop of the device penetrated the distal cap. A guidewire was advanced across the penetrated distal cap and into the distal true lumen.

The peripheral device was removed and an Intravascular Ultrasound (IVUS) catheter was introduced over the guidewire, advanced in to the track made by the device passage, and intraluminal disposition of the track was recorded. The IVUS catheter was removed and the target lesion was treated either with a balloon, stent, and/or atherectomy, at the discretion of the operator.

Cine-angiography captured the entire procedure.

Patients were monitored overnight and followed through discharge and at 30 days and 6-months post procedure.

### Enrollment

Up to 10 patients were enrolled at 1 site. Patients included men and women over 18 years of age with peripheral CTO lesions in the lower limbs having a Rutherford Clinical Classification Categories 2-5 and a life expectancy greater than 6 months at the time of enrollment.

There were a number of exclusion criteria, such as infection, planned intervention during the study, bleeding diseases, allergies to the materials used, recent myocardial infarction or stroke, prior procedures or amputations on the limb, and various other criteria.

### Inclusion Criteria

The following inclusion criteria were followed:
1. Subject was a suitable candidate for angiography and endovascular intervention per ESC guidelines I/TASC II recommendations.
2. Target lesion(s) was located in a native de novo common femoral (CFA), superficial femoral (SFA) or popliteal arteries.
3. Target lesion(s) reference vessel diameter was between 3.50 mm and 8.0 mm by visual estimate.
4. Target lesion(s) were ≥20 mm and ≤200-mm in length
5. Target lesion(s) were CTO, 100% occluded per investigator visual assessment at time of procedure.
6. Subject had at least one patent tibial vessel on the target leg with run-off to the ankle.
7. No evidence of aneurysm or dissection in target vessel.
8. Calcification of CTO region of lesion was less than severe.

### Outcome Measures

Safety: freedom from device-related Major Adverse Events (MAEs) at 30 days and 6 months post procedure, as per Clinical Events Committee (CEC) adjudication, defined as: need for emergency surgical revascularization of target limb, unplanned limb amputation, dissection of grade D or greater that require an intervention to resolve, or symptomatic distal embolization, defined as clinical signs or symptoms of distal emboli detected in the treated limb distal to the treated lesion after the index procedure or noted angiographically after the index procedure and requiring mechanical or pharmacologic means to improve flow.

Performance/Technical Success: The ability of the catheter to intraluminally cross a CTO of the peripheral arteries of the lower limb and facilitate placement of a guidewire into the distal true vessel lumen, as assessed by IVUS and/or angiography via core lab evaluation.

Procedural Success: Achievement of Technical Success together with post-procedural patency. Post-procedural patency is defined as ≤50% residual percent diameter stenosis at the end of revascularization procedure and at 6 months. Additionally, the ability of the crossing catheter to perform as follows:
j) Pre-treatment assembly of the device over the guidewire and into the guide catheter without damage or kinking.
k) Delivery of the device, guidewire, and guide catheter assembly to the desired location proximal to the proximal cap of the CTO.
l) Extension and expansion of the Centering System out of the guide catheter.
m) Ability of the device to penetrate the proximal cap.
n) Ability of the cutting loop to rotate in response to the rotation of the handle wheel as it bores through the CTO.
o) Ability of the device tip/cutting loop to penetrate the distal cap.
p) Ability to place a guidewire in the true vessel lumen distal to the target CTO.
q) Ability to retrieve the device into the guide catheter.
r) Ability to remove the device from the body and maintain guidewire access.

### Data Analysis

Data was analyzed using statistical methods. Categorical data was summarized using frequency tables, presenting the subject counts and percentages. Continuous variables were summarized by the mean, standard deviation, median, minimum and maximum. Within-subject changes were analyzed parametrically using the Paired t-test if the differences are normally distributed, or non-parametrically using the Sign-Rank Test if the differences are not normally distributed. Sample size was small (10) because this was a first-in-human study.

Results: In the first in-human trial involving six subjects conducted in Paraguay and Panama, the peripheral crossing system (e.g., the devices disclosed herein) demonstrated 75% technical success in crossing femoropopliteal and 100% technical success in tibial CTOs with no device-related adverse events. They created a wide intraluminal channel in all successful subjects without embolization.

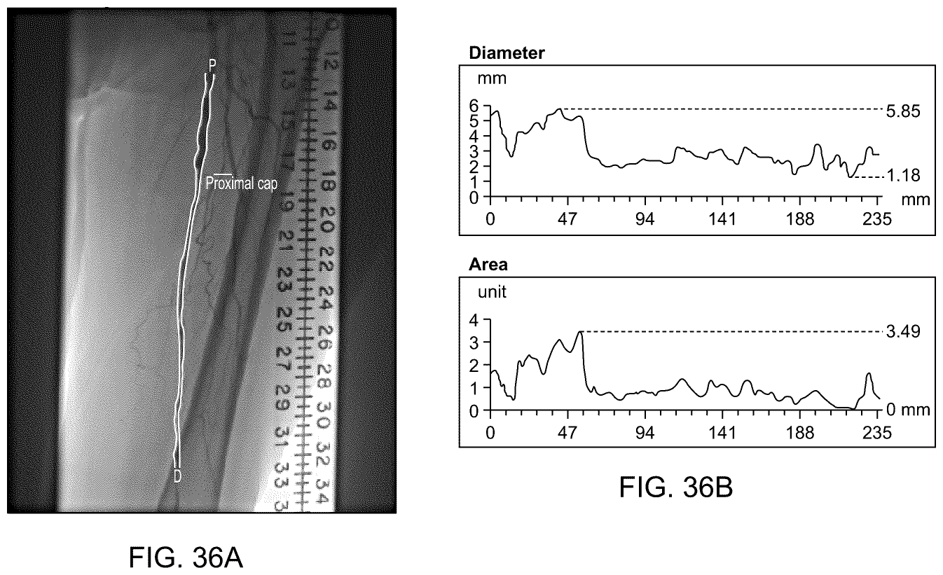

An example of a resulting channel for a patient is shown in FIGS. 36A-36B, as taken via IVUS and presented using a post processing software. Some of the tracts made were larger in diameter than the largest diameter of the device. Surprisingly, the vessels where the tracts were made showed zero, or negligible, distension, expansion, widening, extension, stretching, abrasion, or cutting. Also surprising was that the device showed positive results (e.g., tract widening) in soft, medium, and hard calcification plaque, unlike atherectomy and angioplasty devices that specialize in hard plaque.

The maximum diameter of the tract formed through a previously occluded section of the upper leg was 5.85 mm. In other areas of the tract, the minimal diameter of the tract formed was 1.18 mm. The maximum area of the tract formed through a previously occluded section of the upper leg was 3.49 mm. In other areas of the tract, the minimal area of the tract formed was 0 mm.

Another patient had a maximum tract diameter of 4.96 mm, a minimum diameter of 2.38 mm, and an average of 3.67 mm below the knee. This patient's cross sectional maximum area was 19.34 mm², the minimum area was 4.44 mm², and the average was 11.89 mm².

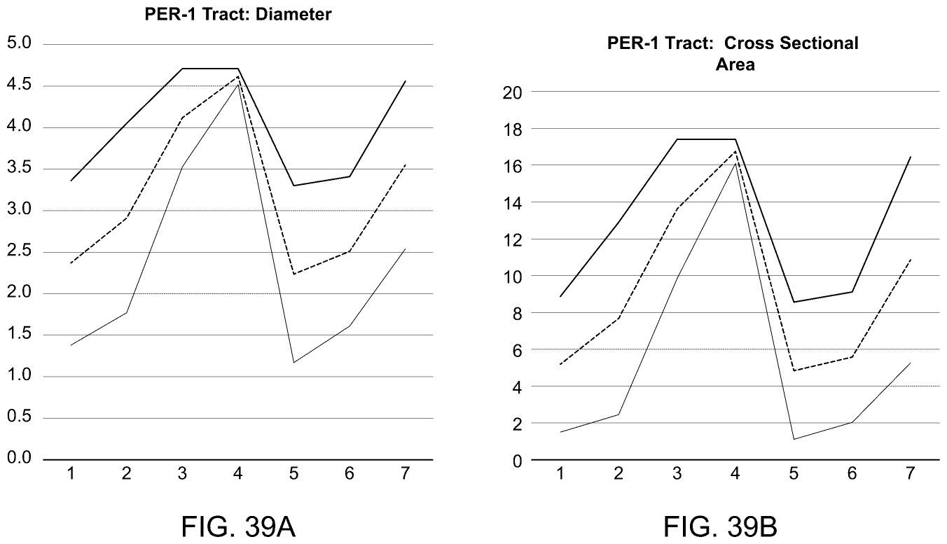

FIGS. 39A-39B illustrate graphs of channel diameters and cross-sectional areas, respectively, resulting from passage of a device through an occlusion as described herein as it crossed through an occlusion using methods described in examples 2 and 3. These graphs show combined results from the two studies. These graphs show occlusions in the femoropopliteal arteries. The x-axis represents the number of patients (7). The y-axis represents the tract diameter in FIG. 39A and the tract cross-sectional area in FIG. 39B after use of a device described herein. The darkest line illustrates the maximum value results of each subject across three trials. The medium gray line illustrates the mean value results of each subject across three trials. The lightest line illustrates the minimum value results of each subject across the three trials.

As shown in FIG. 39A, across all of the participants in the trials of examples 2 and 3, the single largest area of tract diameter was 4.71 mm. The single smallest area of tract diameter was approximately 1.17 mm. The average of the areas with the largest tract diameters was 4.01 mm. The average of the areas with the mean value results of the tract diameters was 3.19 mm. The average of the areas with the smallest tract diameters was 2.36 mm.

As shown in FIG. 39B, across all of the participants in the trials of examples 2 and 3, the single largest area of tract cross section was 17.4 mm². The single smallest area of tract cross section was 1.11 mm. The average of the areas with the largest tract cross sections was 12.96 mm². The average of the areas with the mean value results of the tract cross sections was 9.21 mm². The average of the areas with the smallest tract cross sections was 5.47 mm².

### Example 4: Developing and Using an Embolic Protection Device

A mold/wire assembly can be programmed by heat-treatment to around 5000 F for about 10-15 seconds, which may conform a Nitinol wire to its austenite phase shape, memorized by its lattice network. The wire can be programmed to have a cone spiral at the distal end, followed by a pig tail coil at the distal-most about 5 cm. Then the device can be cooled to room temperature (around 20-25 C) at which Nitinol returns to its masternite phase. If this preprogrammed Nitinol device/wire is exposed to a temperature at or above its “transition temperature” (=<30 degrees C., which is close to human body temperature) the device can regain it preprogrammed austenite phase shape. The nitinol wire can retain its superelastic properties over a broad (up to 500 C) range over its transition temperature.

The device/wire can be manually straightened in its the masternite phase and introduced inside the central lumen of a micro catheter or any interventional device as described herein and deployed inside the blood vessel along with micro catheter/interventional device. The entire distal 20-25 cm length of the device/wire can remain constrained in the central lumen of the catheter/interventional device until the targeted lesion is completely crossed and microcatheter/interventional device tip is in the blood vessel lumen distal to the target lesion. At this point the device/wire can be pushed distally out of central lumen (thus releasing from its constrained state) into the vessel lumen distal to the lesion. When the device/wire get exposed to the transition temperature it immediately regains its memorized austenite phase shape. At this point, the embolic protection device can be used to capture any debris released while the targeted lesion was crossed or any subsequent debris from further widening, cutting, and/or crossing.

### Example 5: GLP Animal Study

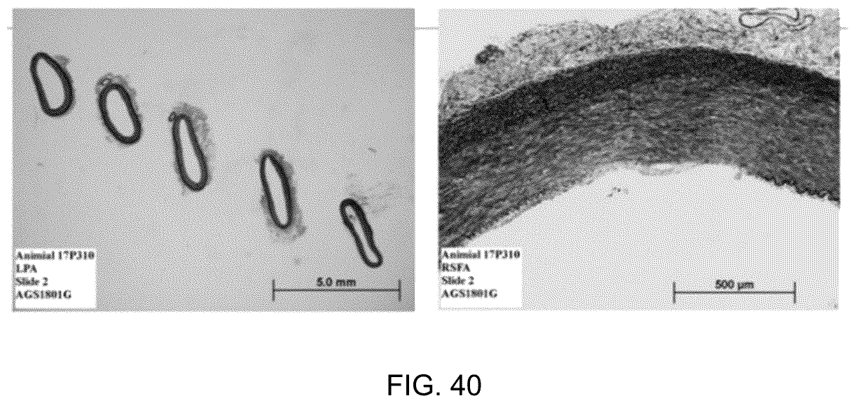

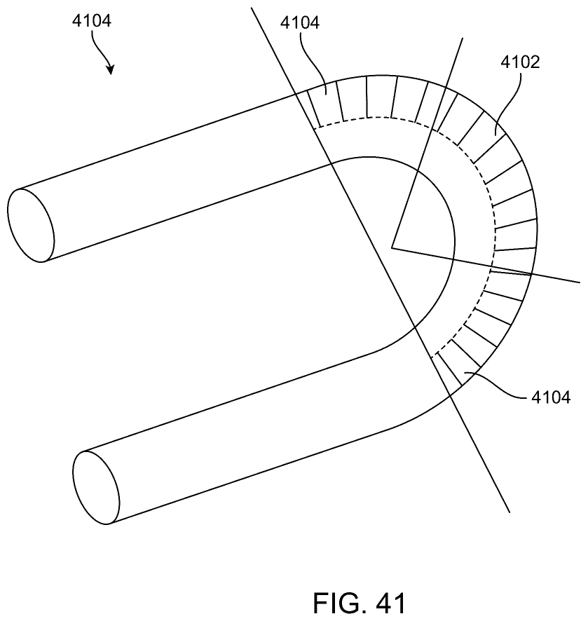

A Pre-GLP Pilot study and GLP study were conducted prior to human trials. The studies tested the device of FIGS. 3A-3B for crossing of chronic total occlusions (CTOs) in the peripheral arteries of lower limbs. The device was the test device, and the control device was an existing CTO catheter. Arterial histology of the test device versus the control device showed substantial equivalency (FIG. 40) in both the pilot and GLP studies. The gross and histological assessment was taken at 3 days (subacute) and 30 days (chronic). Distal organ thromboembolic risk was evaluated.

Results: The predominant pathology included minimal to mild “wall contact” endovascular lesions affecting intima (endothelial loss, intimal edema or hyperplasia). There was less frequency of internal elastic lamina (IEL) fracture or sub-intimal compression. There was no fibrin, no thrombus, no perforation, no dissections, and no inflammation.

### Example 6: Tip Load and Penetration Power Comparison

Tip load and penetration power of the devices disclosed herein were measured and compared to third-party devices. FIG. 41 shows the tip of a cutting loop 4100 of a device as described herein. The tip of the cutting loop can be separated into the forward cutting surface 4102 and the lateral plaque displacing surface 4104. Both 4102 and 4104 can add to the tip load of the device. Forward cutting surface 4102 also determines the penetration power of the device.

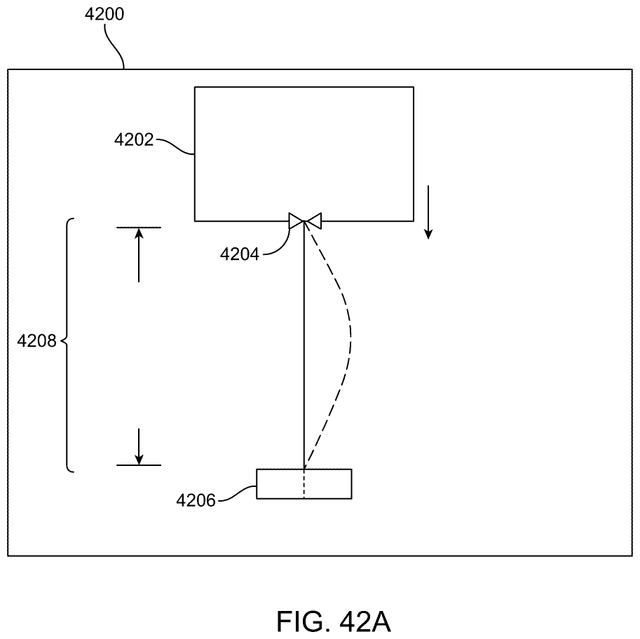

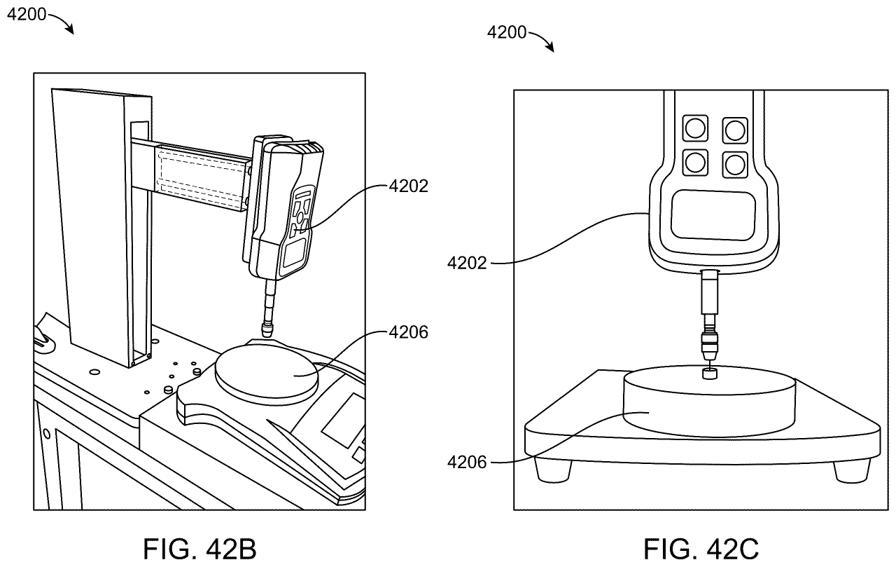

Tip load and penetration power were measured as shown in FIGS. 42A-42C using setup 4200. FIGS. 42A-42C show force test stands 4202, device fixation point 4204, silicone reference and base 4206, and test sample distance 4208. The silicone reference had a 2 mm radius. Test sample distance 4208 was varied to measure the tip load and penetration power of the devices tested. Test sample distance 4208 was varied between 5 mm, 10 mm, and 15 mm. The force test stand 4202 was moved down vertically until the test sample device buckled approximately 2 mm, using the silicone reference 4206 for the buckling distance. The dotted curve shown in FIG. 42A represents the curve of a buckled device.

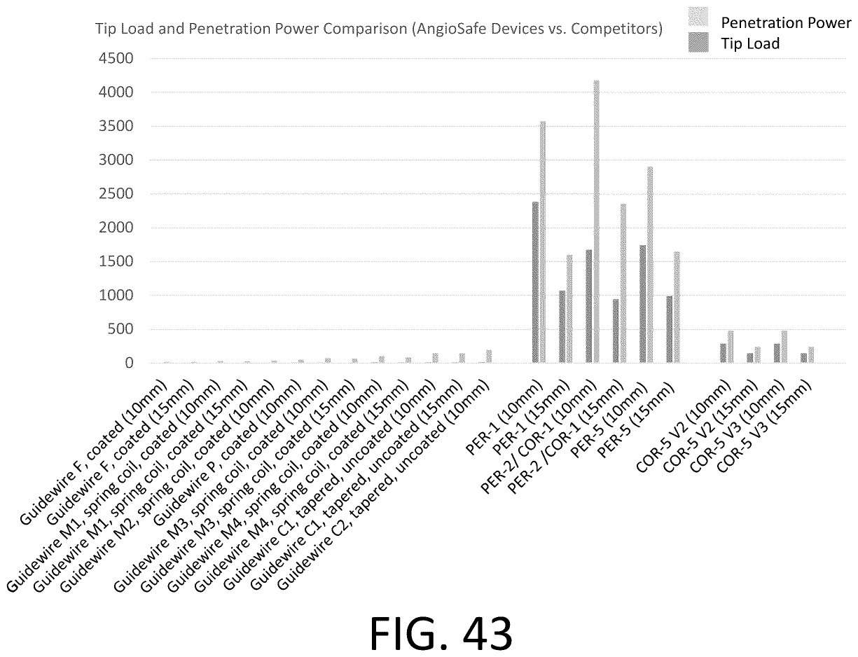

The devices disclosed herein, especially those in FIGS. 2A-8 and 26A-26G were compared against 8 third-party devices and recorded in Table 1 below. Table 1 is presented in graphical form in FIG. 43.

TABLE 1
Tip Load and Penetration Power Comparison of Devices

| | Tip load | Penetration Power |
|---|---|---|
| Device/GuideWire | (gm) | (Kg/sq in) |
| Guidewire F, coated (10 mm) | 1.2 | 19 |
| Guidewire F, coated (15 mm) | 1.07 | 17 |
| Guidewire M1, spring coil, coated (10 mm) | 3.9 | 32 |
| Guidewire M1, spring coil, coated (15 mm) | 3.50 | 29 |
| Guidewire M2, spring coil, coated (10 mm) | 4.4 | 36 |
| Guidewire P, coated (10 mm) | 8 | 52 |
| Guidewire M3, spring coil, coated (10 mm) | 8.9 | 72 |
| Guidewire M3, spring coil, coated (15 mm) | 5.25 | 66 |
| Guidewire M4, spring coil, coated (10 mm) | 13 | 106 |
| Guidewire M4, spring coil, coated (15 mm) | 10.17 | 85 |
| Guidewire C1, tapered, uncoated (10 mm) | 9.3 | 145 |
| Guidewire C1, tapered, uncoated (15 mm) | 9.27 | 145 |
| Guidewire C2, tapered, uncoated (10 mm) | 12.4 | 194 |
| PER-1 (10 mm) | 2382 | 3573 |
| PER-1 (15 mm) | 1068 | 1602 |
| PER-2/COR-1 (10 mm) | 1672 | 4180 |
| PER-2/COR-1 (15 mm) | 942 | 2355 |
| PER-5 (10 mm) | 1742 | 2903 |
| PER-5 (15 mm) | 988 | 1647 |
| COR-5 V2 (10 mm) | 286 | 477 |
| COR-5 V2 (15 mm) | 144 | 240 |
| COR-5 V3 (10 mm) | 286 | 477 |
| COR-5 V3 (15 mm) | 144 | 240 |

The COR-5 and COR-1 are the devices as shown in FIGS. 6A-8 with twin cutting loops, and can be designed for coronary vessels due to the thinner nature of the device. The PER 1, 2, and 5 devices are variations of FIGS. 2A-5.

As shown in Table 1, the tip load of the devices disclosed herein was between about 11.6 times larger (comparing the lowest tip load in the cardiovascular devices disclosed herein against the largest tip load of the third-party devices) to about 2,226 times larger (comparing the highest tip load in the peripheral devices disclosed herein against the smallest tip load of the third-party devices). Similarly, the penetration power was between about 1.2 times larger (comparing the lowest tip load in the cardiovascular devices disclosed herein against the largest tip load of the third-party devices) to about 245 times larger (comparing the highest tip load in the peripheral devices disclosed herein against the smallest tip load of the third-party devices). The larger penetration powers and tip loads in the cardiovascular and peripheral devices disclosed herein can allow for penetration of hard, calcified plaques that third-party devices may not penetrate.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the invention and that methods and structures within the scope of these claims and their equivalents be covered thereby.

1. A method for treating an occluded artery, the method comprising:
(i) centering a working end of a treatment catheter in the occluded artery, wherein centering comprises maintaining a position of the working end within a lumen of the occluded artery;
(ii) disrupting an occlusion mass with the working end of the treatment catheter, wherein disrupting the occlusion mass comprises pushing the working end through the occlusion mass with a tip load force of from about 50 g to about 2400 g;
(iii) radially displacing the disrupted occlusion mass and adjacent occlusion mass toward a wall of the occluded artery, wherein radially displacing the disrupted and adjacent occlusion mass comprises:
(a) creating a blood flow channel in the occlusion mass while simultaneously maintaining the centering of the working end of the treatment catheter,
(b) enlarging a diameter of the channel by compressing the disrupted and adjacent occlusion mass toward a wall of the artery while minimizing injury to the artery wall; and
(iv) withdrawing the treatment catheter from the artery after the channel is enlarged,
wherein the diameter of the channel is enlarged by at least 30% by compressing the disrupted and adjacent occlusion mass toward the wall of the artery.

2. The method of claim 1, wherein centering the working end comprises deploying a centering element of the treatment catheter.

3. The method of claim 2, further comprising advancing the treatment catheter through the occluded artery, wherein the deployed centering element radially displaces the disrupted occlusion mass as the treatment catheter is advanced through the occluded artery.

4. The method of claim 2, wherein the centering element is positioned at or near the working end of the treatment catheter.

5. The method of claim 2, wherein the centering element comprises one or more of an expandable anchoring balloon, at least one flat spring, or at least one cutting loop.

6. The method of claim 5, wherein centering the working end comprises oscillating or rotating the at least one cutting loop.

7. The method of claim 2, wherein the centering element comprises at least one flat spring disposed circumferentially about a distal portion of a tubular catheter body of the treatment catheter and adapted to elastically self-expand from a radially constrained configuration to a radially expanded configuration wherein the at least one flat spring has a wide lateral surface configured to atraumatically interact with a wall region of the artery to centrally align the tubular catheter body in a lumen of the artery.

8. The method of claim 7, wherein disrupting the occlusion mass comprises penetrating a narrow distal edge on the at least one flat spring through the occlusion mass to enlarge the channel through the occlusion mass as the catheter is advanced.

9. The method of claim 7, wherein at least one flat spring has an Ω-shaped profile with a base portion attached to the tubular catheter body and a loop portion extending radially away from the tubular catheter body.

10. The method of claim 5, wherein the at least one flat spring comprises a plurality of flat springs.

11. The method of claim 1, wherein disrupting the occlusion mass with the working end of the treatment catheter comprises cutting the occlusion mass with a cutting element at the working end of the treatment catheter.

12. The method of claim 11, wherein cutting the occlusion mass with the cutting element comprises rotating or oscillating the cutting element.

13. The method of claim 12, wherein the cutting element comprises a cutting tip, at least one cutting loop, or both.

14. The method of claim 13, wherein disrupting the occlusion mass comprises one or more of: pushing the cutting tip mounted on a distal end of the catheter, or rotating the cutting tip, independently or simultaneously.

15. The method of claim 13, wherein the at least one cutting loop comprises two or more parallel cutting loops, two or more crossed cutting loops, at least one centered cutting loop, at least one cutting loop curved to a side, or any combination thereof.

16. The method of claim 13, wherein the cutting tip comprises a fluted tip, a pointed-end tip, a serrated tip, a helical screw tip, a tubular tip, a conical tip, a screw surface tip, an eyelet tip comprising a guidewire, or any combination thereof.

17. The method of claim 1, wherein withdrawing the catheter comprises withdrawing the catheter over a guidewire disposed in a lumen of the treatment catheter, wherein the guidewire remains in the channel left behind by the distal end of the catheter and in the artery lumen.

18. The method of claim 17, further comprising extending the guidewire past an end of the treatment catheter into the artery after radially displacing the occlusion mass, wherein extending the guidewire causes it to return to a shape memory form, wherein the shape memory form creates an embolic protection device comprising a spiral cone filter and a distal coil.

19. The method of claim 18, further comprising capturing debris released while disrupting the occlusion mass in the spiral cone filter.

20. The method of claim 1, wherein the treatment catheter comprises an interventional catheter, a delivery catheter, a guide catheter, a microcatheter, an embolic protection device, or any combination thereof.

21. The method of claim 1, wherein disrupting the occlusion mass causes substantially no dissection, cutting, perforation, damage, injury, stretching, abrasion, or any combination thereof to the artery.

22. The method of claim 1, wherein radially displacing the disrupted and adjacent occlusion mass causes substantially no expansion, widening, extension, stretching, abrasion, cutting, or any combination thereof to the artery.

23. The method of claim 22, wherein radially displacing the disrupted and adjacent occlusion mass causes substantially no expansion, widening, extension, stretching, abrasion, cutting, or any combination thereof due to one or more of a substantially negligible strain rate, substantially negligible lateral force application, or substantially negligible active application of lateral strain rate.

24. The method of claim 1, wherein disrupting the occlusion mass comprises applying one or more of mechanical, vibrational, ultrasound, thermal, cooling, cryo, or optical energy to the occlusion mass.

25. The method of claim 1, wherein radially displacing the disrupted and adjacent occlusion mass creates a channel with a diameter greater than or approximately matching the widest part of the working end of the treatment catheter.

26. The method of claim 1, wherein radially displacing the disrupted and adjacent occlusion mass creates a channel in the occluded artery having a diameter from about 0.5 mm to about 5 mm.

27. The method of claim 1, wherein radially displacing the disrupted and adjacent occlusion mass comprises creating a channel in the occlusion mass having a cross sectional area from about 2 mm² to about 18 mm².

28. The method of claim 1, wherein the enlargement of the channel improves channel diameter by about 1 mm to about 4 mm in a vessel size of from about 1.5 mm to about 15 mm.

29. The method of claim 1, wherein the enlargement of the channel provides percentage improvement in channel diameter from about 5% to about 80%.

30. (canceled)

31. The method of claim 1, wherein the working end has a degree of freedom from a longitudinal axis of the treatment catheter.

32. The method of claim 31, further comprising deflecting the working end of the treatment catheter via the degree of freedom along an orbital or elliptical path.

33. The method of claim 32, wherein disrupting the occlusion mass comprises disrupting the occlusion mass such that a diameter of the disruption of the occlusion is based on the deflection of the working end.

34. The method of claim 33, wherein, due to the degree of freedom and the deflection of the working end of the treatment catheter, the enlarged channel is greater than a maximum diameter of the treatment catheter.

35. The method of claim 34, wherein the enlarged channel is greater than the maximum diameter of the treatment catheter by from about 10% to about 20%.

36. The method of claim 34, wherein the enlarged channel is greater than the maximum diameter of the treatment catheter by at least about 20%.

37. The method of claim 33, wherein the diameter of the enlarged channel is, after withdrawing the treatment catheter, greater than a maximum diameter of the treatment catheter due to the deflection of the working end of the treatment catheter.

38. The method of claim 1, wherein the occlusion mass comprises calcification, wherein disrupting the occlusion mass comprises penetrating the occlusion mass with the working end, wherein the working end has a penetration power of at least 200 kg/sq in.

39. The method of claim 38, wherein the working end has a penetration power of at least 400 kg/sq in.

40. The method of claim 1, wherein the occluded artery comprises an occluded lower extremity artery.

41. The method of claim 1, wherein the occluded artery comprises an occluded coronary artery.

42. The method of claim 1, wherein the occluded artery comprises an above-the-knee artery.

43. The method of claim 1, wherein the occluded artery comprises a below-the-knee artery.

44. The method of claim 1, wherein the occluded artery comprises a femoropopliteal artery.

45. The method of claim 1, wherein the occluded artery comprises an iliac artery.

46. The method of claim 1, wherein the occluded artery comprises a femoral artery.

47. The method of claim 1, wherein the occluded artery comprises a popliteal artery.

48. The method of claim 1, wherein the occluded artery comprises a pedal artery.

49. The method of claim 1, wherein the occluded artery comprises a radial artery.

50. The method of claim 1, wherein the occluded artery comprises a carotid artery.

51. The method of claim 1, wherein the working end comprises a cutting end, one or more compression elements, and a base, wherein the cutting end and the one or more compression elements are coupled to the base.
